(19) United States
(12) Patent Application Publication
(10) Pub. No.: US 2025/0255603 A1
(43) Pub. Date: Aug. 14, 2025

(54) BARBED DEVICES FOR SECURING TISSUE

(71) Applicant: Axogen Corporation, Alachua, FL (US)

(72) Inventors: Kiri K. HAMAKER, Tampa, FL (US); Nikunj Kumar AGRAWAL, Tampa, FL (US); Nesreen ALSMADI, Alachua, FL (US); Benjamin SPEARMAN, Alachua, FL (US); Robert DILUCCIO, Alachua, FL (US); Justin DEUERLING, Alachua, FL (US); Curt DEISTER, Alachua, FL (US)

(73) Assignee: Axogen Corporation, Alachua, FL (US)

(21) Appl. No.: 19/047,021

(22) Filed: Feb. 6, 2025

Related U.S. Application Data
(60) Provisional application No. 63/737,119, filed on Dec. 20, 2024.
(60) Provisional application No. 63/721,964, filed on Nov. 18, 2024.
(60) Provisional application No. 63/683,523, filed on Aug. 15, 2024.
(60) Provisional application No. 63/551,695, filed on Feb. 9, 2024.

(51) Int. Cl. A61B 17/06

(52) U.S. Cl. CPC A61B 17/06166; A61B 2017/06176

(57) ABSTRACT

A barbed suture may include a suture body and at least one multi-directional barb formed on an outer surface of the suture body. The at least one multi-directional barb includes a first projection and a second projection, the first projection and the second projection extending in two different directions. In another embodiment, a barbed suture may include a suture body and at least two barbs formed on an outer surface of the suture body. Each of the at least two barbs has a first projection pointing in a first direction, and a second projection pointing in a second direction, the first direction being different than the second direction. The invention also relates to methods of forming barbed sutures.

### CROSS REFERENCE TO RELATED APPLICATIONS

This patent application claims the benefit of priority to U.S. Provisional Patent Application No. 63/551,695, filed on Feb. 9, 2024; U.S. Provisional Patent Application No. 63/683,523, filed on Aug. 15, 2024; U.S. Provisional Patent Application No. 63/721,964, filed on Nov. 18, 2024; and U.S. Provisional Patent Application No. 63/737,119, filed on Dec. 20, 2024, the entireties of each of which are incorporated herein by reference.

### FIELD OF THE INVENTION

The present disclosure relates generally to the field of barbed devices for the securing and fixing of tissue, and, more specifically, to barbed sutures, barbed constructs, barbed mesh, and barbed strips for the securing and fixing of tissue to other tissue and/or to other devices, and related methods of manufacture and use.

### BACKGROUND

Fixation of tissue, such as nerve tissue, to other tissue or to devices includes placement of sutures. Suture placement may be dependent on technique. With fixation of nerve tissue, ideal placement of a suture would be in the epineurium of the nerve tissue, so that the internal structures of the nerve tissue are generally undisrupted. Such ideal placement of a suture is not always achieved. In addition, perfect placement of a suture may depend on an angle of approach, or the location or orientation of an intended connection between tissue, e.g., nerve ends, or between tissue and a device. These variables may make it difficult for a surgeon to achieve perfect placement of a suture. Further, existing sutures may not be ideal for preventing movement of sutured tissue in more than one direction.

For example, epineurial suture neurorrhaphy is widely acknowledged as the gold standard for peripheral nerve repair and reconstruction. This technique, however, requires significant expertise in microsurgical methods. Achieving proper micro suture placement and precise approximation of nerve ends are tasks that demand a high level of skill and are both time-intensive and technically challenging. Penetration of the suture needle beyond the outermost epineurial layer could result in the damage to the critical fascicular structures of the nerve.

Given these inherent complexities, there is growing need for alternative tissue repair strategies, such as for nerves, that do not rely on sutures. Such innovations could significantly enhance the efficiency of nerve or other tissue repair procedures, e.g., by reducing the time required and simplifying the technical demands placed on surgeons.

### SUMMARY

In one aspect of the present disclosure, a barbed suture may include a suture body and at least one multi-directional barb formed on an outer surface of the suture body, the at least one multi-directional barb including a first projection and a second projection, wherein the first projection and the second projection extend in two different directions.

In another aspect of the present disclosure, a barbed suture may include a suture body, and at least two barbs formed on an outer surface of the suture body, a first barb of the at least two barbs having a first projection pointing in a first direction, and a second barb of the at least two barbs having a first projection pointing in a second direction, wherein the first direction is different from the second direction.

In still another aspect of the present disclosure, a method of making a barbed suture may include making a first cut into a body of a suture to form a first projection oriented in a first direction, wherein the first cut does not extend fully through the body of the suture, and making a second cut into the body of the suture to form a second projection oriented in a second direction, wherein the second cut does not extend fully through the body or the suture, wherein the first direction is different from the second direction.

In still another aspect of the present disclosure, a tissue repair device may include at least one mesh comprising a plurality of barbed sutures, including at least a plurality of longitudinal barbed sutures, and optionally a plurality of lateral barbed sutures, each barbed suture including a suture body and at least one barb formed on an outer surface of the suture body.

In still another aspect of the present disclosure, a tissue repair device may include a base membrane and a plurality of barbs formed on the base membrane, the plurality of barbs being formed by one of laser cutting or embossing.

In still another aspect of the present disclosure, a tissue repair device may include a base membrane and a plurality of barbs formed on the base membrane, the plurality of barbs being formed of resorbable ceramic and being attached to the base membrane.

In still another aspect of the present disclosure, a tissue repair device may include a base membrane and at least two barbed sutures, a longitudinal length of the barbed sutures being greater than a width of the base membrane, each barbed suture including portions having barbs and a portion that is free from barbs.

In still another aspect of the present disclosure, a tissue repair device may include a strip of base membrane and at least two barbed sutures attached to the strip of base membrane.

In still another aspect of the present disclosure, a method of securing two tissue ends relative to one another may include affixing the two tissue ends to a tissue repair device comprising at least one mesh formed of a plurality of barbed sutures, wherein the plurality of barbed sutures includes a plurality of longitudinal barbed sutures and optionally a plurality of lateral barbed sutures, each barbed suture including a suture body and at least one barb formed on an outer surface of the suture body, and wrapping an outer surface of each of the two tissue ends in the tissue repair device.

In still another aspect of the present disclosure, a method of securing two tissue ends together may include affixing the two tissue ends to a tissue repair device comprising a base membrane and a plurality of barbs formed on the base membrane, the plurality of barbs formed by one of laser cutting and embossing, and wrapping an outer surface of the two tissue ends in the tissue repair device.

In still another aspect of the present disclosure, a method of securing two tissue ends together may include affixing the two tissue ends to a tissue repair device comprising a base membrane and a plurality of barbs attached to the base membrane, the plurality of barbs being formed of resorbable ceramic, and wrapping an outer surface of each of the two tissue ends in the tissue repair device.

In still another aspect of the present disclosure, a method of securing two tissue ends together may include affixing the two tissue ends to a tissue repair device comprising a base membrane and at least two barbed sutures, a longitudinal length of the barbed sutures being greater than a width of the base membrane, each barbed suture comprising portions including barbs and a portion that is free from barbs, and wrapping an outer surface of each of the two tissue ends in the tissue repair device.

Other objects, features and advantages of the present disclosure will become apparent from the following detailed description. It should be understood, however, that the detailed description and the examples, while indicating exemplary embodiments of the present disclosure, are given by way of illustration only, since various changes and modifications within the spirit and scope of the invention will become apparent to those skilled in the art from this detailed description.

In this disclosure, the singular forms “a,” “an,” and “the” include plural referents unless the context dictates otherwise. The term “exemplary” is used in the sense of “example” rather than “ideal.” The terms “comprises,” “comprising,” “includes,” “including,” or other variations thereof, are intended to cover a non-exclusive inclusion such that a composition, method, or process that comprises a list of elements or steps does not necessarily include only those elements or steps, but may include other elements or steps not expressly listed or inherent to such a composition, method, or process. The relative terms, such as “approximately” and “about,” are generally used to indicate a possible variation of ±10% of a stated or understood value unless indicated otherwise in the specification. In addition, the term “between” used in describing ranges of values is intended to include the minimum and maximum values described herein. The use of the term “or” in the claims and specification is used to mean “and/or” unless explicitly indicated to refer to alternatives only or the alternatives are mutually exclusive, although the disclosure supports a definition that refers to only alternatives and “and/or.” As used herein “another” may mean at least a second or more.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of exemplary embodiments presented herein.

FIG. 1 is a schematic view of a portion of a non-barbed suture and a blade.

FIG. 2 is a schematic view of a portion of a suture with a barb formed thereon.

FIG. 3 is a schematic view of a portion of a suture with more than one barb formed thereon.

FIG. 4 is a schematic view of a portion of a suture with a multi-directional barb formed thereon.

FIGS. 5 to 11 are schematic views of portions of barbed sutures having barbs of varying shapes, according to various embodiments.

FIGS. 12 to 20 are schematic views of portions of barbed sutures having barbs of varying arrangements, according to various embodiments.

FIG. 21 is a schematic view of a barbed suture having a plurality of barbs on a surface thereof.

FIG. 22 is a detail view of two adjacent barbs of the barbed suture shown in FIG. 22.

FIG. 23 is FIG a schematic view of a barbed suture having a number of barbs on a surface thereof.

FIG. 24 depicts a detail view of three adjacent barbs of the barbed suture shown in FIG. 23.

FIGS. 25, 26, and 27 are schematic detail views of the barbed suture shown in FIGS. 23 and 24.

FIG. 28 is a schematic view of a barbed suture having a number of barbs on a surface thereof, FIG. 29A depicts a detail view of three adjacent barbs of the barbed suture shown in FIG. 28, and FIG. 29B is an image of tissue ends with barbed sutures, as in FIGS. 28 and 29A, attached thereto.

FIG. 30 is a schematic view of a dual plane barbed suture having a number of barbs on a surface thereof, FIG. 31 depicts a detail view of three adjacent barbs of the barbed suture shown in FIG. 30, in one plane and three adjacent parts in another plane, and FIG. 32 depicts a cross-sectional view of the barbed suture shown in FIG. 30.

FIGS. 33 and 34 are images of an example barbed suture, described above with respect to FIGS. 30 to 32.

FIG. 35 is a schematic drawing of a barbed suture with two adjacent barbs, formed using mechanical cutting.

FIG. 36 is a schematic drawing of a barbed suture, with two adjacent barbs, formed using injection micro molding.

FIG. 37 depicts a flowchart of a method of forming a barbed suture, according to one or more embodiments.

FIG. 38 is a flow chart of a method of forming a barbed suture, according to one or more embodiments.

FIG. 39 is a schematic depiction of a base membrane, which may be used in forming a tissue repair device according to the embodiments described herein.

FIG. 40 shows a detail view of a base membrane, a barbed structure 4005, such as a portion of a barbed suture or a portion of a barbed device, and a layer of a polymer, as a glue or an adhesive, therebetween.

FIGS. 41 and 42 are images showing the barbed sutures adhered to a base membrane during testing.

FIG. 43 shows a base membrane with a barbed structure on top of the base membrane, and a spot welding probe.

FIG. 44 is a detail view of an interface between a thermally bonded barbed structure and a base membrane.

FIG. 45 is a detail of an alternative interface between a thermally bonded structure and a base membrane.

FIG. 46 shows one embodiment in which barbed sutures are weaved through a base membrane.

FIG. 47 shows another embodiment in which barbed sutures are sewn through a base membrane.

FIG. 48 depicts a base membrane with barbed sutures attached using cuts or hooks in the base membrane.

FIG. 49 depicts a base membrane with barbed sutures being threaded through holes in the base membrane.

FIG. 50 shows a back side of the base membrane and barbed sutures 4905 shown in FIG. 49.

FIG. 51 depicts barbed sutures attached to a base membrane by inserting the base membrane into a thin slit or cut through the barbed sutures.

FIG. 52 is a detail view of a slit of the barbed suture shown in FIG. 51.

FIG. 53 depicts another embodiment in which barbed sutures are attached to a base membrane by inserting the base membrane into a thin slit or cut through the barbed sutures.

FIG. 54 is a detail view of a slit of the barbed suture shown in FIG. 53.

FIG. 55 depicts barbed sutures attached to layers of base membrane by sandwiching barbed sutures between layers of base membrane.

FIG. 56 depicts a top layer of base membrane having holes provided in a particular pattern or arrangement, used to sandwich the barbed sutures with additional layers of base membrane while allowing barbs to extend through the top layer of base membrane.

FIGS. 57, 58A, 58B, and 59 are schematic cross-sectional views of barbed sutures on base membranes.

FIGS. 60, 61A, and 61B are images of several examples of tissue repair devices having barbed sutures attached to base membranes.

FIGS. 62 and 63 depict tissue repair devices having base membranes and barbed sutures.

FIG. 64A is a schematic view of a barbed mesh, according to one embodiment, and FIGS. 64B and 64C are images of a barbed mesh that is pre-formed and rolled.

FIG. 65 is a schematic view of a configuration of the barbed mesh shown in FIG. 64, during a tissue repair process, according to one embodiment.

FIG. 66 is a schematic view of a configuration of the barbed mesh shown in FIGS. 64A and 65, with a base membrane, as shown in FIG. 39, during a tissue repair process, according to another embodiment.

FIG. 67 is a schematic view of a configuration of the barbed mesh shown in FIGS. 64A and 65, with a base membrane, as shown in FIG. 39, during a tissue repair process, according to still another embodiment.

FIG. 68 is a schematic view of another barbed mesh, as part of a tissue repair device, with no base membrane.

FIG. 69 shows the tissue repair device of FIG. 68, with a strip of base membrane laid atop the barbed mesh, according one embodiment, with tissue ends.

FIG. 70 is a schematic view of a base membrane with barbs.

FIG. 71 is a schematic view of a barbed mesh on a strip of base membrane, according to one embodiment.

FIG. 72 is a schematic view of a configuration of two barbed meshes on strips of base membrane, as shown in FIG. 71, during a tissue repair process, according to one embodiment.

FIG. 73 is a schematic view of a configuration of two barbed meshes on strips of base membrane, as shown in FIG. 71, and a base membrane, during a tissue repair process, according to another embodiment.

FIG. 74 is a schematic view of a configuration of the barbed meshes on the strips of base membrane, as shown in FIG. 71, and a base membrane, during a tissue repair process, according to still another embodiment.

FIG. 75 is a schematic view of an injection molded strip of base membrane with barbs, according to one embodiment.

FIG. 76 is a schematic view of a strip of base membrane with ceramic barbs, according to another embodiment.

FIG. 77 is a schematic view of two barbed sutures and a base membrane, according to one embodiment.

FIG. 78 is a schematic view of two sets of side-by-side barbed sutures, and a base membrane, according to another embodiment.

FIG. 79 is a schematic view of two strips or meshes with barbed sutures, and a base membrane, according to yet another embodiment.

FIG. 80 is a schematic view of the two strips with barbed sutures and the base membrane, shown in FIG. 79, during a tissue repair process, according to one embodiment.

FIG. 81 is a schematic view of two strips or meshes with barbed sutures adhered to a first base membrane, and a second base membrane, according to still another embodiment.

FIG. 82 is a schematic view of a mesh of barbed sutures adhered to a base membrane, according to one or more embodiments.

FIG. 83 is a schematic view of the mesh of barbed sutures adhered to the base membrane, as shown in FIG. 82, during a tissue repair process, according to one embodiment.

FIG. 84 is a schematic view of the mesh of barbed sutures adhered to the base membrane, as shown in FIG. 82, during a tissue repair process, according to another embodiment.

FIG. 85 is a schematic view of a strip with barbed attachment points, according to one embodiment.

FIG. 86 is a schematic view of two strips with barbed attachment points, as shown in FIG. 85, during a tissue repair process, according to one embodiment.

FIG. 87 is a schematic view of two strips with barbed attachment points, as shown in FIG. 85, and a base membrane, during a tissue repair process, according to another embodiment.

FIG. 88 is a schematic view of two strips with barbed attachment points and a base membrane, during a tissue repair process, according to another embodiment.

FIGS. 89 to 91 are schematic views of a tissue repair device formed of a strip of base membrane and a barbed mesh, according to another embodiment.

FIGS. 92, 93, and 94 show another embodiment of a tissue repair device, formed of a strip of base membrane and a barbed mesh, according to another embodiment.

FIGS. 95, 96, and 97 show another embodiment of a tissue repair device, formed of a base membrane having a large tab and a small tab, and a plurality of barbed sutures.

FIGS. 98, 99, and 100 show another embodiment of a tissue repair device, formed of a base membrane having a large tab and a small tab, and a barbed mesh.

FIG. 101 shows another embodiment of a tissue repair device, formed of a base membrane and a plurality of barbed sutures.

FIG. 102 shows another embodiment of a tissue repair device, formed of a base membrane and a plurality of barbed sutures.

FIG. 103 shows another embodiment of a tissue repair device, formed of a base membrane and a barbed mesh.

FIG. 104 shows another embodiment of a tissue repair device, formed of a base membrane and a barbed mesh.

FIG. 105 is a schematic view of a tissue repair device including a plurality of barbed sutures mounted or embedded within a base membrane, according to another embodiment.

FIG. 106 shows a plurality of barbed sutures with barbs, an integrated strip of base membrane, and two transected tissue ends.

FIG. 107 is a schematic view of a tissue repair device having a base membrane with internal slits or openings, and longitudinally arranged barbed sutures, and FIG. 108 is a schematic view of a tissue repair device having a base membrane with external slits or openings, and longitudinally arranged barbed sutures.

FIGS. 109 to 111 are images of a tissue repair device and the tissue repair device in use, as in FIG. 105.

FIG. 112 is a schematic view of a tissue repair device according to still another embodiment.

FIG. 113 is a detail view of the micro-injection molded barbs.

FIG. 114 depicts the tissue repair device of FIG. 112, wrapped around tissue ends.

FIGS. 115 to 118 show side cross-sectional views of strips and barbs.

FIG. 119 is a flowchart of a method of securing two tissue ends together, according to one or more embodiments.

FIG. 120 is a flowchart of an additional step of the method shown in FIG. 119.

FIG. 121 is an image of glass fibers extending from a resorbable polymer surface.

### DETAILED DESCRIPTION

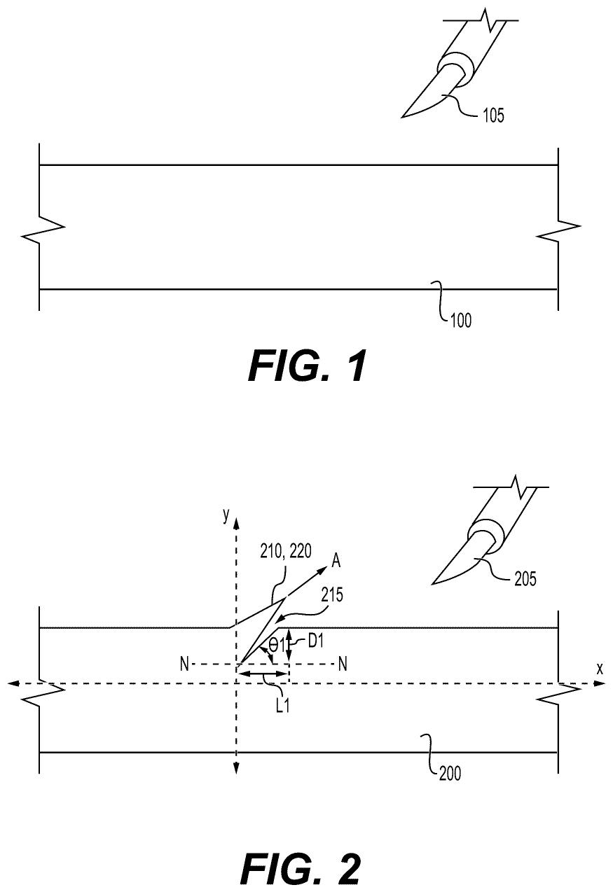

Barbed sutures may be used for fixation of tissue. Existing barbed sutures may include barbs formed by cutting or slicing portions of a suture, as shown in FIGS. 1 and 2. An outer circumferential surface of a suture may be cut such that barbs are created on the outer surface. The cut may not extend all of the way through the suture, and the cut may be angled, rather than orthogonal to the surface of the suture. In traditional barbed sutures, the cuts are made so that all of the barbs are oriented, or point, in one direction. Current barbed sutures are designed to assist in wound closure and to allow for the barbed suture to attach to soft tissues and to resist movement in one direction. Existing barbed sutures are intended for wound fixation and do not have ideal physical characteristics for fixation to tissue, e.g., nerve tissue, to itself, to other tissue, or to a device. In addition, existing barbed sutures are intended to fix tissues to resist movement in only one direction: counter to the direction of the barbs (such as the direction A in FIG. 2). Such barbed sutures may detach from tissue when moved in directions other than the one direction against which the barbs on the sutures are oriented to resist movement.

### I. Overview

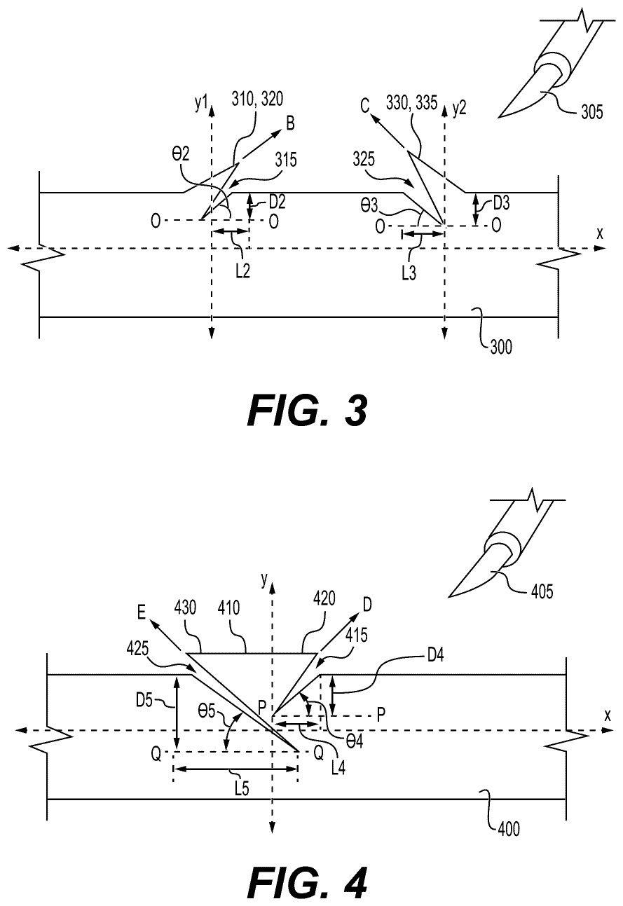

Embodiments of the present disclosure may include a barbed suture having a plurality of barbs oriented in different directions. As shown in FIGS. 3 and 4, and as described in more detail below, exemplary sutures may be formed by cutting barbs at different angles relative to one another. In the example shown in FIG. 3, a suture may be cut at least once to form at least one barb oriented in a first direction, and then cut at another location to form at least one other barb oriented in a second direction. That is, in one embodiment, a barbed suture has a plurality of barbs with only one projection, with the projections of at least two barbs pointing in two different directions. Such barbed sutures may resist movement in two directions. In other embodiments, different barbs may be oriented in three or more directions. Barbs with projections pointing in different directions may be organized in a repeating pattern, may be grouped regularly, or barbs with projections pointing in different directions may be oriented randomly in different directions along the suture.

In the example shown in FIG. 4, a suture may be cut to form a barb having a first projection oriented in a first direction, and then a cut may be made in the vicinity of the first cut, in a different direction from the first cut, to form a second projection of the same barb, the second projection being oriented in a second direction. In these embodiments, the barbed suture formed by multiple cuts, that is, the multi-directional suture and/or the multi-directional barbs, may provide a barbed suture that is capable of rapid attachment to tissue from any angle of approach or connection. Also, the barbed suture may be capable of resisting movement in multiple angles of direction and/or multiple directions when the barbed suture is attached to tissue(s), and, therefore, may also resist or reduce detachment from the tissue(s).

Embodiments of the present disclosure may include a tissue repair device including a plurality of barbed sutures. In some aspects, one or more barbed sutures may be affixed to or used with a base membrane. In some examples, the tissue repair device may comprise a mesh formed of barbed sutures. In some aspects, the barbed sutures mesh may be affixed to or used with a base membrane. In some aspects, the barbed sutures may comprise portions with and without barbs, depending on how the tissue repair device is designed to be attached to tissue, to multiple tissue ends, or to a device.

In some aspects, the tissue repair device may be wrapped around or otherwise attached to two tissue ends. In some examples, the tissue ends may physically touch one another, or there may be a gap between the tissue ends when the tissue repair device is in use. The gap between the two tissue ends may, for example, allow for the tissue to retract in vivo in response to changing tension during the rescission procedures.

Although transected tissue ends are discussed in the disclosure, the tissue repair devices of the embodiments described, e.g., with respect to FIGS. 2 to 114, may be applied to injured tissues of any type, whether fully transected or not, e.g., for protection or to allow the underlying tissue to heal, or simply to secure tissue for any other suitable reason, unrelated to healing. For example, instead of two tissue ends being brought together, embodiments of the disclosure may wrap around or otherwise cover a portion of tissue that is intact, to promote repair or to otherwise protect the underlying tissue.

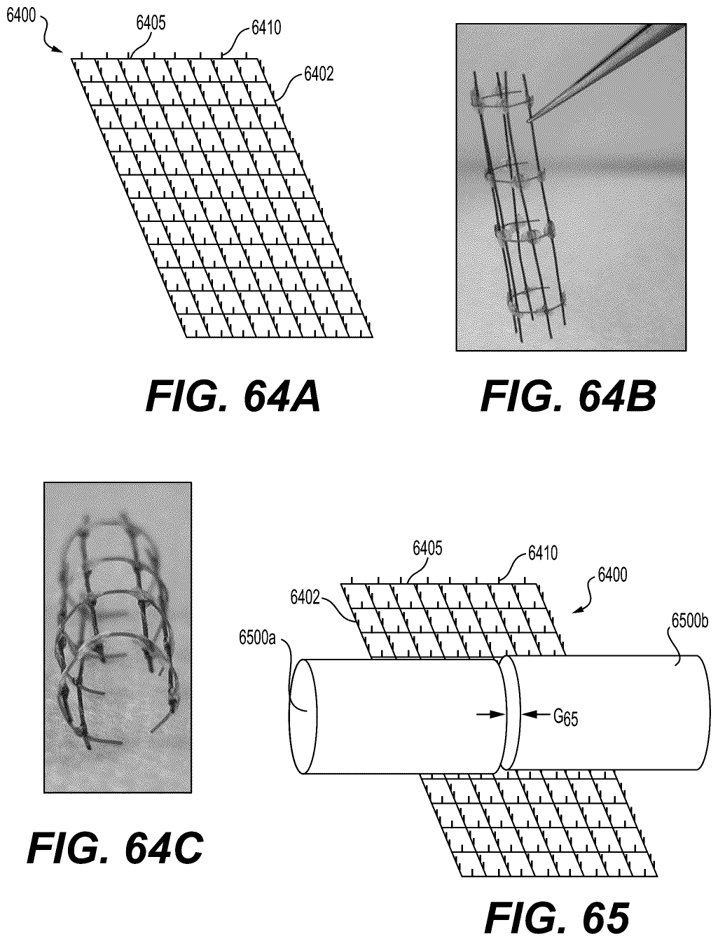

In examples shown in FIGS. 64 to 118, a tissue repair device may consist of strips of barbed sutures, which may or may not be connected to or used with a base membrane, wherein the base membrane is wrapped around or otherwise applied to the two tissue ends.

Embodiments of the present disclosure may include barbed devices and methods for fabricating barbed devices. In some instances, the resulting barbed devices may be described as a sutureless tissue coaptation devices, or more specifically, sutureless nerve coaptation devices, although barbed devices of the present disclosure may be used with any suitable tissue, not just nerves. Sutureless, as used herein, refers to not requiring the use of suturing with surgical sutures that are separate from the devices described in the disclosure.

### II. Barbed Suture

FIG. 1 is a schematic view of a portion of a non-barbed suture 100 (also referred to as a suture body) and a blade 105. The non-barbed suture 100 may be used to form the barbed sutures or barbed devices described herein. The suture 100, and, therefore, the barbed sutures and barbed devices described herein, may be formed of one or more polymers. The one or more polymers may be a resorbable polymer that biodegrades over time. The one or more polymers may comprise homopolymers, copolymers, and/or polymeric blends including one or more of the following monomers: glycolide, lactide, caprolactone, dioxanone, trimethylene carbonate, monomers of cellulose derivatives, and monomers that polymerize to form polyesters. The polymer may include polydioxanone (PDO), polylactic acid (PLA), polycaprolactone (PCL), polytrimethylene carbonate, polyglycolide (PGL), polyglycolic acid (PGA), poly-3-hydroxybutyrate (PHB), poly(3-hydroxybutyrate-co-3-hydroxyvalerate) (PHBV), poly(4-hydroxybutyrate) (P4HB), poly(propylene carbonate) (PPC), poly(butylene succinate) (PBS), poly(propylene fumarate) (PPF), polydroxyalkanoates, copolymers thereof, or one or more other suitable materials. In some examples, the one or more polymers may comprise polyester, polydioxanone (e.g., poly-p-dioxanone), poliglecaprone (such as poliglecaprone 25 sold under the tradename Monocryl®), polyglytone (such as polyglytone 6211 sold under the tradename Caprosyn®), catgut, collagen, polyglycolide, polylactide (e.g., poly-L-lactide). In some aspects, the suture 100 may be absorbable. In still other examples, the barbed sutures may be formed of biodegradable metal.

In some examples, the tip of the barbed suture may have a height designed to inhibit damage to the tissue to which it is applied. For example, in the case of use with nerve tissue, the tip of the barbed suture may be designed to be less than a thickness of the nerve epineurium, in order to inhibit the potential for damage to the critical fascicular structures of the nerve tissue. For example, the tip of the barbed suture may be less than about 500 μm, less than about 400 μm, less than about 300 μm, less than about 200 μm, less than about 100 μm, less than about 50 μm, less than about 30 μm, or less than about 1 μm. The tip of the barbed suture may be about 1 μm to about 100 μm, about 1 μm to about 200 μm, about 1 μm to about 300 μm, about 1 μm to about 400 μm, about 1 μm to about 500 μm, about 10 μm to about 400 μm, about 10 μm to about 300 μm, about 10 μm to about 200 μm, about 10 μm to about 100 μm, etc.

FIG. 2 is a schematic view of a barbed suture 200, that may be formed of the non-barbed suture 100 shown in FIG. 1, with a first barb 210 formed in the suture by cutting or scoring a circumferential surface of the suture 200 with a blade 205, or, in other words, making a first cut 215, thereby forming the barbed suture 200. The first cut 215 has a depth D1 relative to a vertical y-axis, and a length L1 relative to a horizontal x-axis, and is formed at an angle θ1 relative to a longitudinal axis N-N of the suture. The first cut 215 forms the barb 210 and, in particular, forms a first projection 220 of the barb, pointing in a direction A. By virtue of the first projection 220 of the barb 210, the barbed suture 200 may be capable of resisting movement or displacement in the direction A relative to tissue(s) to which it is attached, and, therefore, the barbed suture 200 may be capable of resisting or reducing detachment from the tissue(s) in response to a force pulling the barbed suture 200 along the x-axis in the direction that the barb 210 is pointing.

FIG. 3 is a detail schematic view of a surface of a barbed suture 300, similar to the barbed suture 200 shown in FIG. 2, with a first cut 315 and a second cut 325 made in an outer surface of the suture 300 using a blade 305. As with the embodiment shown in FIG. 2, the first cut 315 may have a depth D2 relative to a y1-axis, and a length L2 relative to the x-axis, and may be formed at an angle θ2 relative to a longitudinal axis O-O of the suture 300. The first cut 315 may form a first barb 310 having a first projection 320 pointing in a direction B. The second cut 325 may be made at another location in the outer surface of the suture 300, and may have a depth D3 that is the same as the depth D2 of the first cut 315, and a length L3 that is the same length L2 of the first cut 315. Alternatively, the depth D3 of the second cut 325 may be greater than or less than the depth D2 of the first cut 315, and/or the length L3 of the second cut 325 may be greater than or less than the length L2 of the first cut 315. In some aspects, the second cut 325 may be formed at an angle θ3 that is the same angle θ2 of the first cut 315, or the angle θ3 of the second cut 325 may be greater than or less than the angle θ2 of the first cut 315. The second cut 325 may form a second barb 330 having a second projection 335 pointing in a direction C, which differs from the direction B in which the first projection 320 points. In this example, a component (horizontal component parallel to the x-axis in FIG. 3) of direction C is different, e.g., opposite, to a component (horizontal component parallel to the x-axis in FIG. 3) of direction B, while other components (vertical components parallel to the y1-axis and the y2-axis in FIG. 3) of the directions C and B are the same.

In some aspects, the two cuts 315 and 325 may have the same depth or different depths, and the same lengths or different lengths. In some aspects, the two cuts 315 and 325 may be formed so as to be oriented 180° from each other, or they may be formed so as to be oriented at an angle greater than or less than 180° from each other.

By virtue of the barbed suture 300 having multiple barbs (e.g., a first barb 310 and a second barb 330), with projections (projection 320 and projection 335) pointing in different directions (directions B and C), the barbed suture 300 may resist movement or displacement in both of the directions. And, by virtue of the barbed suture 300 having multiple projections in at least two different directions (directions B and C), the barbed suture 300 may be a multi-directional barbed suture, more specifically, in the embodiment shown in FIG. 3, a bi-directional barbed suture. Although the multi-directional barbed suture 300 shown in FIG. 3 has two barbs 310 and 330, the suture 300 may have more than two barbs. Further, the particular directions in which the projections of the more than two barbs point may vary, e.g., the barbs may be uni-directional with the exception of one barb with a projection pointing in a different direction, or a portion of the barbs may have projections pointing in a first direction, with the remainder of the barbs having projections pointing in a second, different direction. Still further, a multi-directional barbed suture may include three or more barbs with the projections of the three or more barbs pointing in three or more different directions.

The multi-directional barbed suture 300 of this embodiment may be capable of rapid attachment between the barbed suture and tissue from different angles of approach or connection. The multi-directional barbed suture 300 may be capable of resisting movement in multiple angles of direction and/or multiple directions when the barbed suture 300 is attached to tissue(s), and, therefore, may also resist or inhibit detachment from the tissue(s). Multi-directional barbs may be useful, e.g., when coapting two portions of tissue, such as two ends of a nerve, separately allowing the severed ends to come together or remain at a specified distance from each other, as is described further herein.

FIG. 4 is a detail schematic view of a surface of a barbed suture 400, similar to the suture 200 shown in FIG. 2, with a first cut 415 and a second cut 425 formed in the suture 400 using a blade 405. As with the embodiment shown in FIG. 2, the first cut 415 may have a depth D4 relative to the y-axis, and a length L4 relative to the x-axis, and may be formed at an angle θ4 relative to a longitudinal axis P-P of the suture 400. The first cut 415 may form a barb 410 having a first projection 420 pointing in a direction D.

The second cut 425 made in the suture 400 may have a depth D5 that is greater than the depth D4 of the first cut 415. In some aspects, the second cut 425 may have a length L5 that is greater than the length L4 of the first cut 415. The second cut 425 may be formed at another angle θ5 relative to a longitudinal axis Q-Q of the suture 400. The other angle θ5 of the second cut 425 may be the same as, greater than, or less than the angle θ4 of the first cut 415. The second cut 425 may extend into the suture 400 such that, relative to the vertical y-axis in FIG. 4, the second cut 425 undermines the first cut 415 at the relatively greater depth D5. The second cut 425 may be made relatively close to or in the vicinity of the first cut 415, and may form part of the barb 410 and, in particular, may form a second projection 430 of the barb 410, pointing in a direction E.

Although the first, shallower cut 415 is described as being formed before the second, deeper cut 425 is formed, the barbed suture 400 may also be formed by making the first cut 415 to a relatively greater depth (e.g., D5), and the second cut 425 to a relatively lesser depth (e.g., D4). In some aspects, the two cuts 415 and 425 could have the same depth or different depths, and the same lengths or different lengths. For example, the depth of the first cut 415 may be the same as the depth of the second cut 425, with the depth being in a range of about 0.02 mm to about 0.2 mm, about 0.03 mm to about 0.15 mm, or about 0.05 mm to about 0.12 mm. In addition, the angle θ4 of the first cut 415 and the angle θ5 of the second cut 425 may be the same, and may be in a range of about 5° to about 45°, about 10° to about 40°, about 10° to about 30°, or about 10° to about 20°. In some aspects, the two cuts 415 and 425 may be formed so as to be oriented 180° from each other, or they may be formed so as to be oriented at an angle greater than or less than 180° from each other. By virtue of the second projection 430 of the barb 410, the barbed suture 400 may resist movement or displacement in the direction E. And, by virtue of the barb 410 having multiple projections in at least two distinct directions, here, directions D and E, the barb 410 may be a multi-directional barb, and, more specifically, a bi-directional barb. That is, the multi-directional barbed suture 400 of this embodiment may be capable of resisting movement in multiple angles of direction and/or multiple directions when the barbed suture 400 is attached to tissue(s), and, therefore, may also resist or inhibit detachment from the tissue(s).

Although the barbed sutures 200, 300, and 400 described above may be formed by cutting a suture using a blade, other methods of creating barbs in sutures may be used. For example, variable geometry extrusion may be used, which creates a suture having a varying thickness or a varying geometry. The material from which the suture is formed may be cut or extruded in such a way that projections are created in the suture during variable geometry extrusion. In some aspects, for example, barbs may be separately formed either from the same material as the suture or from a different material of the suture, and then they may be fixed onto the suture using welding, annealing, curing, fastening, bonding, laminating, molding, embossing, imprinting, or adhesion. In some aspects, others means of fixing the barbs to the suture may be used. In some aspects, random or irregular patterns of barbs may be created, such as by roughening a surface of the suture.

And, although the barbed sutures 200, 300, and 400 described above may have projections pointing in two directions, such as in the multi-directional barbed suture 300 or the barbed suture 400 having one or more bi-directional barbs 410, barbs having different geometries may be used.

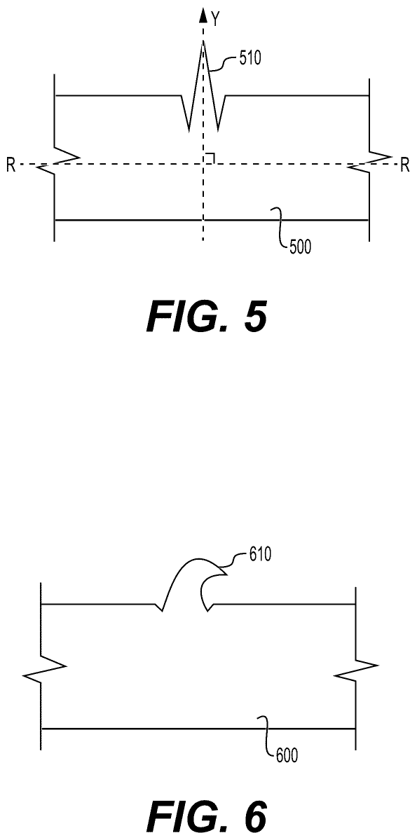

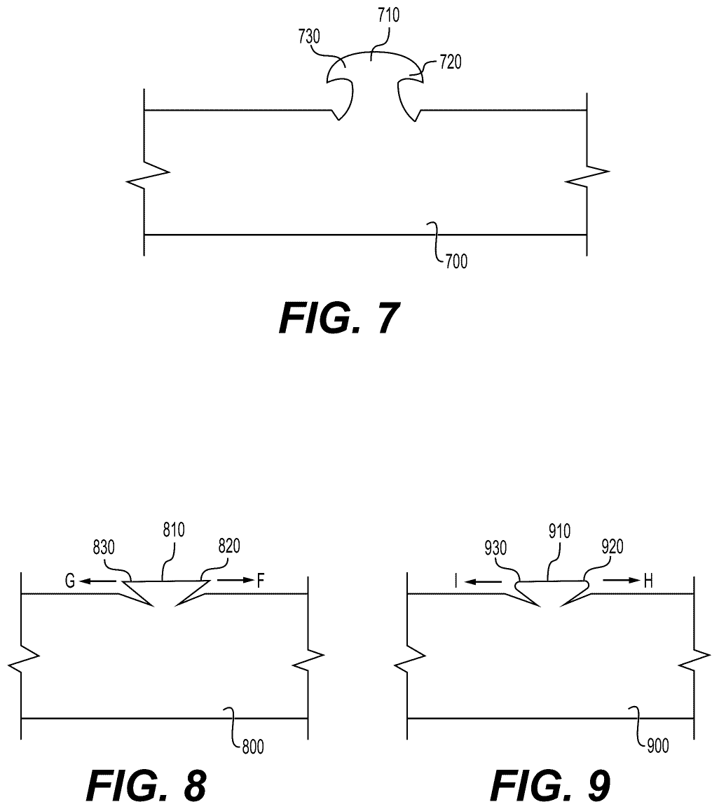

For example, as shown in FIG. 5, a barb 510 on a suture 500 may be a uni-directional barb, which may be oriented approximately perpendicular to a longitudinal axis R-R of the suture 500 (that is, parallel to the y-axis in FIG. 5) to resist movement along the longitudinal axis. In other aspects, a barb 610 may be formed on a suture 600 in a hook shape, as shown in FIG. 6, or a barb 710 may be formed on a suture 700, so as to have multiple hooks, e.g., projections 720 and 730, as shown in FIG. 7. In some aspects, in a case in which a barb 810 is a bi-directional barb formed in a suture 800, the barb 810 may have two projections 820 and 830, facing or pointing in substantially opposite directions F and G, as shown in FIG. 8, or a barb 910 formed on a suture 900 may have two rounded projections 920 and 930, facing or pointing in substantially opposite directions H and I, as shown in FIG. 9. In the embodiments shown in FIGS. 5 to 9, as with the embodiments shown in FIGS. 3 and 4, the two cuts used to form each suture may have the same depth or different depths, and the same lengths or different lengths. In a case in which the barb is a multi-directional barb, the barb may be composed of more than two pointed projections or more than two rounded projections.

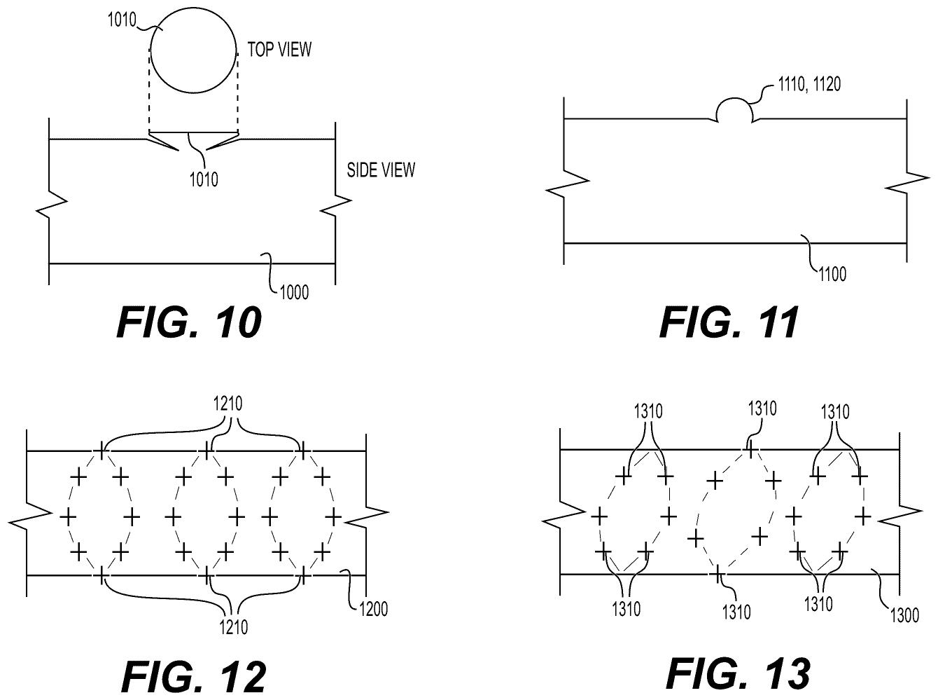

In some aspects, a barb 1010 formed on a suture 1000 may be in the form of a rounded (e.g., oblong or circular) disc, as shown in the multi-plane view of FIG. 10, or a barb 1110 formed in a suture 1100 may be in the form of a single, round or bulbous projection 1120, as shown in FIG. 11. Still further, for example, the barb may be in the form of a geometric shape having three or more sides, e.g., a triangle, a square, a pentagon, a multi-pointed star shape, etc. In some aspects, each barb may have more than two projections. As with the embodiments described above, each of the barbed sutures shown in FIGS. 5 to 11 may be capable of resisting movement in multiple angles of direction and/or multiple directions when the barbed suture is attached to tissue(s), and, therefore, may also resist or reduce detachment from the tissue(s).

Although only one barb is shown on each of the suture in FIGS. 4 to 11, two or more barbs may be formed in the same manner as described above. In some aspects, two or more of the same type of barbs may be included on the suture (e.g., two or more hooked barbs 610 or 710, or two or more bulbous barbs 1110). In other aspects, two or more different types of barbs may be included on the suture (e.g., a mixture of hooked barbs 610 or 710 and bulbous barbs 1110, or a mixture of hooked barbs 610 and 710, bulbous barbs 1110, and bi-directional barbs 810). The barbs may be aligned, for example, in rows, columns, rings, or spirals on the surface of the suture, or in a pattern, such as a grid pattern, or in a random, non-aligned arrangement.

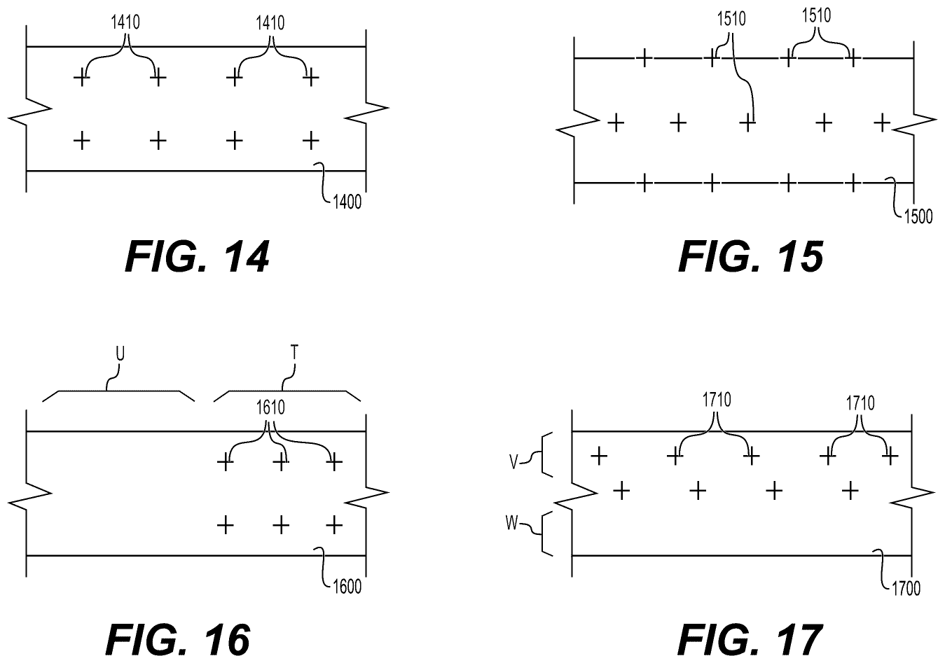

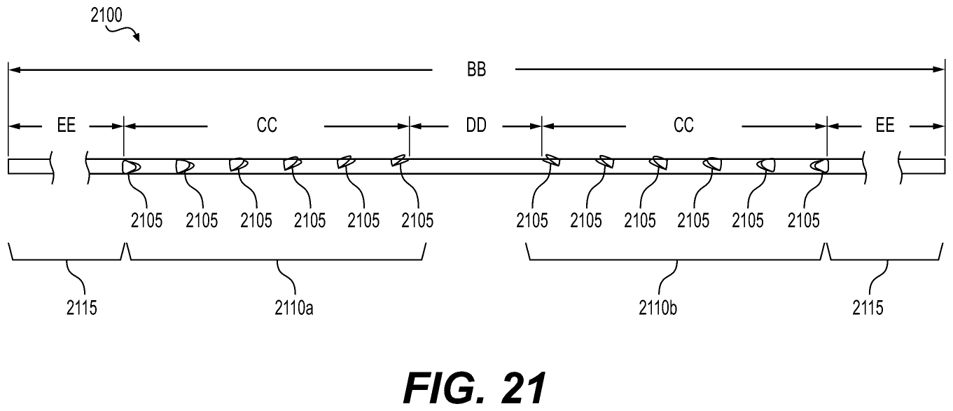

For example, multiple barbs 1210 may be formed on a suture 1200 in a ring around a circumference of the suture 1200. The rings may lie in a plane perpendicular to a longitudinal axis of suture 1200, as shown in FIG. 12, or they may lie in a plane at an angle to the longitudinal axis of the suture, as shown in FIG. 13 with multiple barbs 1310 on a suture 1300. In other embodiments, multiple barbs may be oriented in a spiral along a length of a suture, as opposed to forming discrete rings. In some aspects, multiple barbs 1410 may be formed on a suture 1400 in one or more longitudinal lines along a length of the suture 1400, which may be aligned with one another, as shown in FIG. 14, or multiple barbs 1510 may be formed on a suture 1500 in a longitudinal line, in an offset position, as shown in FIG. 15, along the circumference of the suture. In some aspects, for example, barbs 1610 may be formed on a portion T of a length of a suture 1600, while barbs 1610 may be absent along another portion U of a length of the suture 1600, as shown in FIG. 16. As a result, one end of the suture 1600 may include barbs 1610, while another end may not, or there may be gaps where no barbs 1610 are included along the length of the suture 1600 (as noted below in the discussion of FIG. 21). In other aspects, barbs 1710 may be formed on a portion V of a circumference of a suture 1700, while being absent along another portion W of the circumference of the suture 1700, as shown in FIG. 17. In this way, one side of the suture 1700 may include barbs 1710, while another side of the suture 1700 does not. In some aspects, suture 1700 may include a row of barbs 1710 that extend along one side of suture 1700. Patterns of portions T and U or V and W may alternate or repeat along a length of the respective sutures 1600 and 1700. Still further, barbs may be formed on a suture in a repeating geometric-shaped pattern along the length of the suture (e.g., a triangle pattern, a square or checker pattern, a pentagon pattern, a hexagon or honeycomb pattern, etc.).

In FIGS. 12 to 17, barbs are portrayed as “+” symbols for convenience, but it is anticipated that any suitable type of suture or combinations of suture types—for example, those portrayed and discussed in reference to FIGS. 3 to 11—may be represented by the “+” symbols used in these figures. Further, although the shape of the suture itself (e.g., suture 100, 200) is shown as having a circular cross-section, any suitable cross-sectional shape may be used, e.g., rectangular, triangular, trilobal, pentagonal, hexagonal, etc., as is described further below.

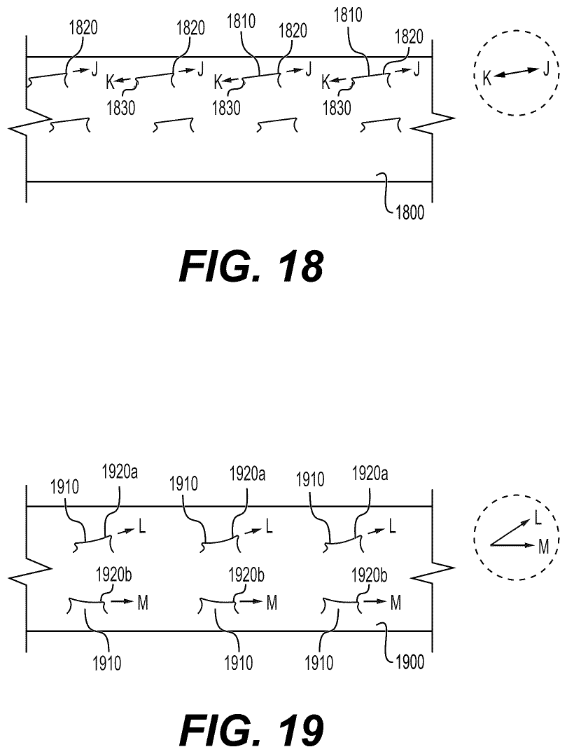

FIG. 18 shows a barbed suture 1800 with multiple multi-directional barbs 1810, with the first projections 1820 of the multi-directional barbs each pointing in the same direction, direction J, for example, or a direction parallel thereto. In some aspects, each of the second projection 1830 of the multi-directional barbs 1810 may be pointing in the same direction, direction K, for example, or a direction parallel thereto, with the directions J and K being substantially opposite to each other, as shown in the detail inset. Alternatively, FIG. 19 shows a barbed suture 1900 with multiple multi-directional barbs 1910 arranged in a pattern, e.g., a regular or irregular pattern, in which a first projection 1920a of each of a first subset of the barbs 1910 points in one direction L, or a direction parallel thereto, and a first projection 1920b of each of a second subset of the barbs 1910 points in another direction M, or a direction parallel thereto. The two directions L and M may be substantially opposite, substantially perpendicular, or, as shown in the detail inset of FIG. 19, angled relative to each other. Similarly, when the multi-directional barbs are arranged in a pattern, a second projection of the first subset of barb may point in one direction, and a second projection of the second subset of bars may point in another direction. The two directions may be substantially opposite, substantially perpendicular, or angled relative to each other.

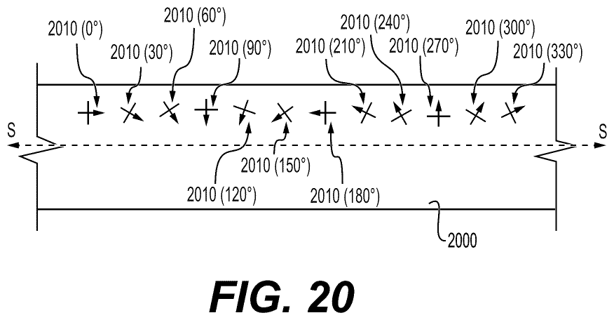

Orientations of barbs along a length of a suture may be arranged in a number of different ways. For example, barbs may be oriented so that they point in a direction that is parallel to a longitudinal axis of the suture, as shown, for example, in FIGS. 8 and 9. Alternatively, for example, the barbs may be oriented so that they point at an angle, relative to a longitudinal axis of the suture, between about 1° to about 359°. In some aspects, as noted above, the barbs may be oriented in the same orientation as adjacent barbs or in a different orientation relative to adjacent barbs. Further, the barbs may have a repeating orientation or random orientations. Still further, as shown in FIG. 20, barbs 2010 on a suture 2000 may have a repeating pattern of orientations relative to a longitudinal axis S-S of the suture 2000. As one particular example of a repeating pattern of orientations, the barbs 2010 in FIG. 20 may be oriented at intervals of about 30° relative to the longitudinal axis S-S of the suture 2000 with each barb 2010 being oriented at an adjusted angle relative to a neighboring barb 2010, in the following order: 0°, 30°, 60°, 90°, 120°, 150°, 180°, 210°, 240°, 270°, 300°, 330°). This pattern may be replicated with any interval of degrees between 0° and 360°, for example, about 20°, about 40°, about 45°, about 50°, about 60°, or about 90° intervals. In some aspects, the barbs may have a non-repeating pattern or an irregular pattern of orientations relative to the longitudinal axis of the suture (i.e., irregular interval of degrees relative to the longitudinal axis of the suture with each sequential barb).

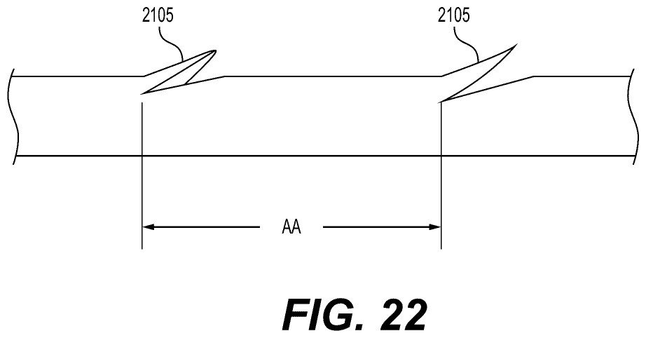

FIG. 21 is a schematic view of a barbed suture 2100 having a number of barbs 2105 on a surface thereof, and FIG. 22 depicts two adjacent barbs 2105 of the barbed suture 2100 shown in FIG. 21, with a spacing AA therebetween. A relatively smaller spacing AA between barbs allows for a relatively greater number of barbs 2105 overall on the barbed suture 2100, and a relatively larger spacing AA between barbs 2105 allows for a relatively lesser number of barbs 2105 overall on the barbed suture 2100. In some aspects, the spacing between adjacent barbs 2105 may remain constant along a length BB of the barbed suture 2100, or the spacing AA between adjacent barbs 2105 may vary along a length BB of the barbed suture 2100. In FIG. 21, barbs 2105 located within segments 2110a and 2110b along lengths CC may be spaced at regular intervals, but there may be a gap DD, or a relatively larger spacing, between the segments 2110a and 2110b of barbs 2105. Ends of the barbed suture, or leaders 2115, may be free from barbs 2105, and may have lengths EE, as shown, or may have barbs 2105. In the embodiment shown in FIG. 21, the barbed suture 2100 has a first segment 2110a of barbs 2105, a second segment 2110b of barbs 2105, and a gap DD between the first segment 2110a and the second segment 2110b.

The barbs 2105 within each segment 2110a, 2110b may be unidirectional (for example, as shown in FIGS. 1, 2, 3, 5, 6, and 11) or multi-directional (for example, bi-directional, as shown in FIGS. 4, 7, 8, and 9) and may point in the same direction as other barbs 2105 (as shown, for example, in FIG. 18) within the same segment 2110a, 2110b, or in different directions from some or all of the other barbs 2105 (as shown, for example, in FIGS. 19 and 20) within the same segment 2110a, 2110b. Further, the barbs 2105 within each segment 2110a, 2110b may have the same angle, relative to the longitudinal axis of the barbed suture 2100, or may have different angles (as discussed and shown, for example, in FIG. 3). In FIG. 21, the barbs 2105 in segment 2110a each face in a similar direction, which is different than the direction of the barbs 2105 in segment 2110b.

The spacing AA between adjacent barbs 2105 may be about 0.01 mm to about 10 mm, e.g., about 0.01 mm to about 5 mm, about 0.01 mm to about 2 mm, about 1 mm to about 7 mm, about 1 mm to about 5 mm, or about 1 mm to about 3 mm. In some aspects, the spacing AA between adjacent barbs 2105 may be about 1 mm or less, e.g., about 0.5 mm to about 1 mm, about 0.4 mm to about 0.8 mm, about 0.4 mm, about 0.5 mm, about 0.6 mm, about 0.7 mm, about 0.8 mm, or about 0.9 mm. The spacing AA between adjacent barbs 2105 may be determined based at least in part on the specific application in which the barbed suture 2100 is to be used.

The overall length BB of the barbed suture 2100 may vary, e.g., depending on use or specific application, but may be about 5 mm to about 1 m, e.g., about 50 mm to about 500 mm, about 100 mm to about 500 mm, about 100 mm to about 300 mm, about 200 mm to about 500 mm, about 200 mm to about 300 mm, for example. The length CC of the segments 2110a, 2110b may be, for example, up to about 50 cm, up to about 30 mm, up to about 10 mm, or up to about 5 mm. In some aspects, the length CC of the segments 2110a, 2110b may be, for example, about 5 mm to about 20 mm, about 5 to about 40 mm, about 10 to about 60 mm, about 40 mm to about 100 mm, about 50 mm to about 90 mm, about 60 mm to about 80 mm, about 60 mm, about 70 mm, or about 80 mm. The length of CC may depend, at least in part, on the overall length of the barbed suture 2100 and/or the intended use or application of barbed suture 2100. The gap DD may be based, at least in part, on a gap length between two tissue portions or between a tissue portion and a device being held in place by the barbed suture 2100. For example, the gap DD may be based, at least in part, on a gap length between two transected nerves (that is, a gap length in nerve transection), or relative desired positioning of two nerve ends, and may be between no or minimal gap to about 80 mm, minimal gap to about 500 mm, minimal gap to about 200 mm, minimal gap to about 100 mm, about 0.5 mm to about 5 mm, about 1 mm to about 4 mm, about 1 mm to about 3 mm, about 0.5 mm, about 1 mm, about 2 mm, about 3 mm, or about 5 mm. The length of gap DD may depend, at least in part, on the overall length of the barbed suture 2100 and/or the intended use or application of barbed suture 2100. The length EE of the leaders 2115 may be about no or minimal gap up to a length of the barbed suture 2100. In some aspects, the length EE of the leaders 2115, if included, may be about 10 mm to about 60 mm, about 20 mm to about 50 mm, about 30 mm to about 40 mm, about 20 mm, about 25 mm, about 30 mm, about 35 mm, about 40 mm, about 45 mm, or about 50 mm, for example. The length EE of leaders 2115, if included, may depend, at least in part, on the overall length of the barbed suture 2100 and/or the intended use or application of barbed suture 2100.

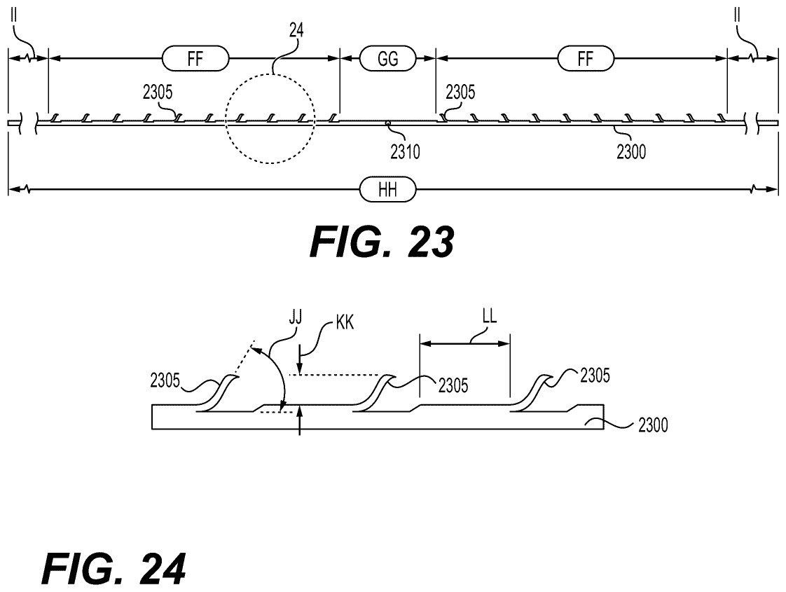

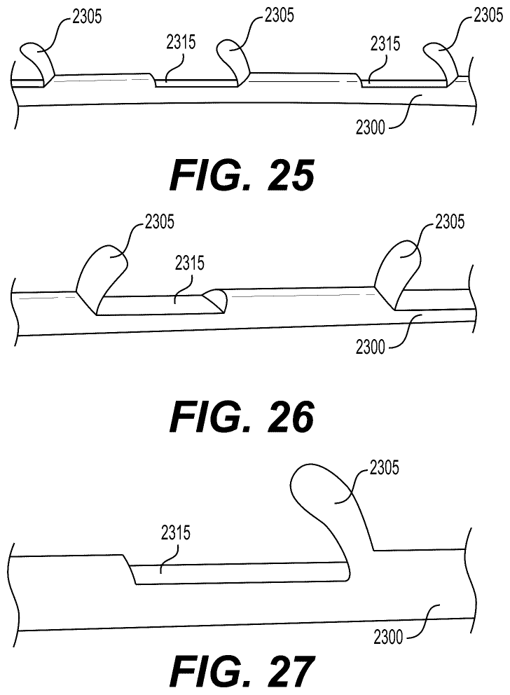

FIG. 23 is a schematic view of a specific example of a barbed suture 2300 having a number of barbs 2305 on a surface thereof, and FIG. 24 depicts a detail view of three adjacent barbs 2305 of the barbed suture 2300 shown in FIG. 23. In the embodiment shown in FIGS. 23 and 24, the barbed suture 2300 may be a size 5-0 gauge suture, a size 4-0 gauge suture, a size 3-0 gauge suture, or other suitable thickness, having a diameter in a range of about 0.100 to about 0.339, about 0.100 to about 0.249 mm, or about 0.150 mm to about 0.199 mm. The barbs 2305 may be arranged with a first set of barbs 2305 on a left side of the barbed suture 2300, shown in FIG. 23, all facing a central region 2310 of the barbed suture 2300, and a second set of barbs 2305 on a right side of the barbed suture 2300, all facing the central region 2310 of the barbed suture 2300. That is, the barbs 2305 may be provided in a bidirectional arrangement. Each of the first set of barbs 2305 and the second set of barbs 2305 spans a length FF, which may be about 15.0±0.5 mm in this example. A gap GG may extend between the first set of barbs 2305 and the second set of barbs 2305, and may have a length of about 5.0±0.5 mm in this example. An overall length HH of the barbed suture 2300 may be any suitable length, depending on its intended use. In this example, the overall length HH may be about 75.0±1.0 mm. In some aspects, the barbed suture 2300 may also include end portions free from barbs 2305, with a length II of the end portions in this example being about 20.0±1.0 mm. With reference to FIG. 24, in this example, an angle JJ between an axis along which a barb 2305 extends and a longitudinal axis, along which the cut, forming the barb 2305 extends, may be about 60°±5°. A height KK of each barb 2305 may be about 0.30±0.03 mm, and a spacing LL between a barb 2305 and a cut for an adjacent barb 2305, as shown in FIG. 24, may be about 0.900±0.1 mm. FIGS. 25, 26, and 27 are schematic detail views of the barbed suture 2300 shown in FIGS. 23 and 24. In particular, FIG. 25 shows three adjacent barbs 2305 along a length of the barbed suture 2300, as well as cuts 2315 made in a surface of the barbed suture 2300 to thereby form the barbs. FIG. 26 shows two adjacent barbs 2305 along a length of the barbed suture 2300, as well as cuts 2315 made in the surface of the barbed suture 2300. And FIG. 27 shows one barb 2305 on the barbed suture 2300, and a cut 2315 made in the surface of the barbed suture 2300 to thereby form the barb 2305.

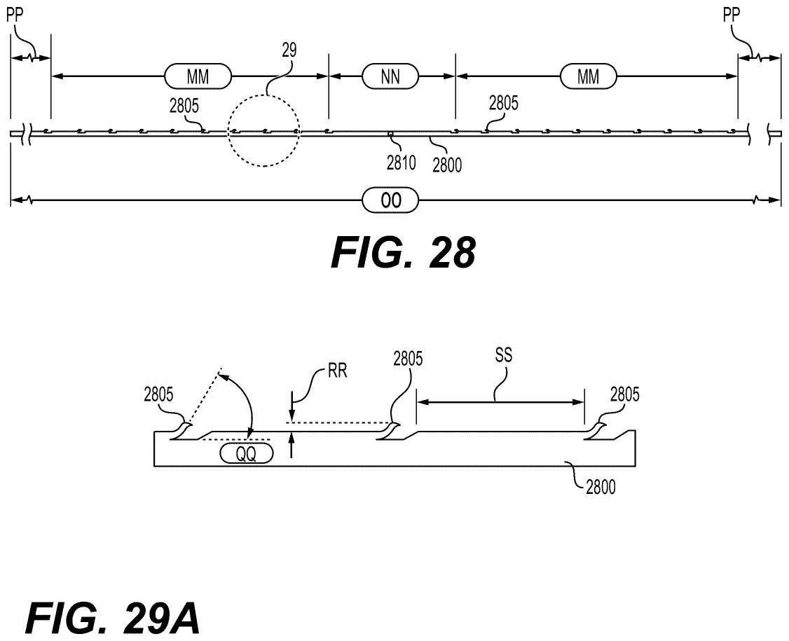

FIG. 28 is a schematic view of another example of a barbed suture 2800 having a number of barbs 2805 on a surface thereof. FIG. 29A depicts a detail view of three adjacent barbs 2805 of the barbed suture 2800 shown in FIG. 28, and FIG. 29B is an image of tissue ends with barbed sutures, as in FIGS. 28 and 29A, attached thereto. The barbs 2805 may be arranged with a first set of barbs 2805 on a left side of the barbed suture 2800, shown in FIG. 28, all facing a central region 2810 of the barbed suture 2800, and a second set of barbs 2805 on a right side of the barbed suture 2800, all facing the central region 2810 of the barbed suture 2800. That is, the barbs 2805 may be provided in a bidirectional arrangement. Each of the first set of barbs 2805 and the second set of barbs 2805 spans a length MM, which may be about 22.5±0.5 mm in this example. A gap NN may extend between the first set of barbs 2805 and the second set of barbs 2805, and may have a length of about 10.0±0.5 mm in this example. An overall length OO of the barbed suture 2800 may be about 95.0±1.0 mm, although any suitable length is acceptable, depending on the intended use of the barbed suture 2800. The barbed suture 2800 may also, in some aspects, include end portions free from barbs 2805, with a length PP of the end portions being about 20.0±1.0 mm, if included. With reference to FIG. 29A, an angle QQ between an axis along which a barb 2805 extends and a longitudinal axis, along which the cut, forming the barb 2805 extends, may be about 60°±5°. A height RR of each barb 2805 may be about 0.10±0.03 mm, and a spacing SS between a barb 2805 and a cut for an adjacent barb 2805, as shown in FIG. 29A, may be about 2±0.1 mm. As noted above, FIG. 29B is an image of tissue ends held in place relative to one another with a plurality of barbed sutures 2800, as in FIGS. 28 and 29A, attached thereto. As shown, a plurality of the barbed sutures 2800 extend longitudinally, parallel to an axis of the tissue ends. Barbs 2805 of the barbed sutures 2800 engage with surfaces of the tissue ends, such that the tissue ends are held together. The gap NN extending between the sections of barbs 2805 may be positioned relative to the tissue ends so as to extend over the terminal ends of the tissues being held together. The first set of barbs may engage the first tissue, and the second set of barbs may engage the second tissue, so that the bi-directional nature of the barbed suture holds the two tissue portions in place relative to one another.

Although transected tissue ends are discussed in reference to FIG. 29B, the tissue repair devices of this embodiment may be applied to injured tissues of any type, whether fully transected or not, e.g., for protection or to allow the underlying tissue to heal, or simply to secure tissue for any other suitable reason, unrelated to healing. For example, instead of two tissue ends being brought together, embodiments of the disclosure may wrap around or otherwise cover a portion of tissue that is intact, to promote repair or to otherwise protect the underlying tissue.

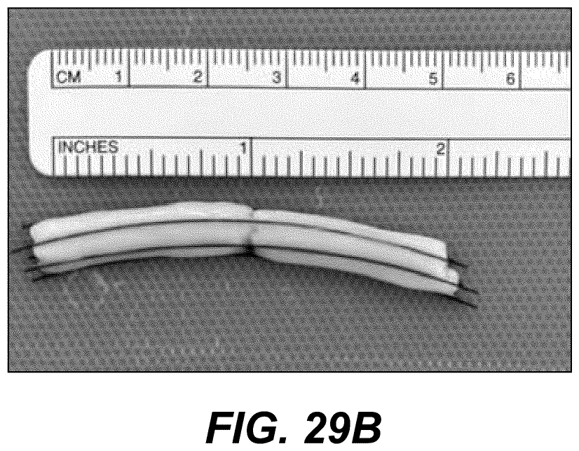

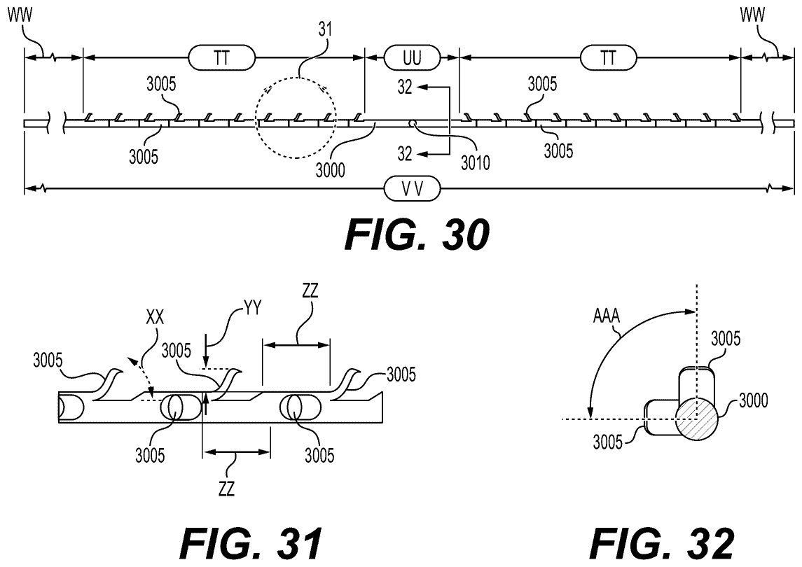

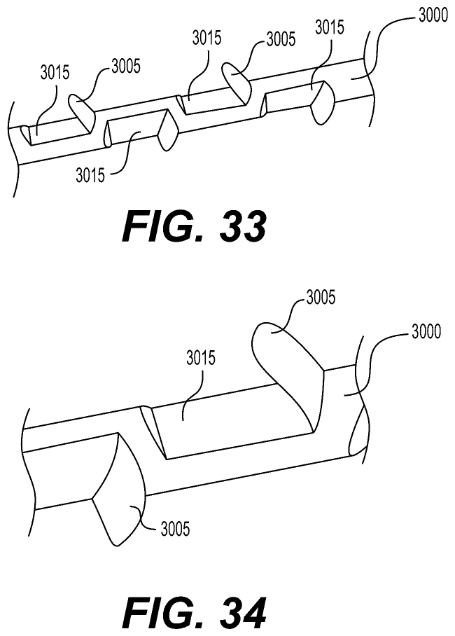

As described above, in some aspects, barbed sutures of the present disclosure may have barbs that are positioned in more than one plane, for example, two planes, three planes, four planes, or more. FIG. 30 is a schematic view of an example dual plane barbed suture 3000 having a number of barbs 3005 on a surface thereof, the barbs 3005 extending in two planes. FIG. 31 depicts a detail view of three adjacent barbs 3005 in one plane and three adjacent barbs 3005 in another plane, and FIG. 32 depicts a cross-sectional view of the barbed suture 3000, with one barb 3005 extending in one plane and another barb 3005 extending in another plane. In the embodiment shown in FIGS. 30 to 32, the barbed suture 3000 may be a size 3-0 suture, having a diameter in a range of about 0.300 mm to about 0.339 mm. In other aspects, barbed suture 3000 may be a size 5-0 gauge suture, a size 4-0 gauge suture, a size 3-0 gauge suture, or other suitable thickness, having a diameter in a range of about 0.100 to about 0.339, about 0.100 to about 0.249 mm, or about 0.150 mm to about 0.199 mm. The barbs 3005 may be arranged with a first set of barbs 3005 on a left side of the barbed suture 3000, shown in FIG. 30, all facing a central region 3010 of the barbed suture 3000, and a second set of barbs 3005 on a right side of the barbed suture 3000, all facing the central region 3010 of the barbed suture 3000. That is, the barbs 3005 may be provided in a bidirectional arrangement. Each of the first set of barbs 3005 and the second set of barbs 3005 spans a length TT. In this example, the length TT may be about 15.0±0.5 mm. A gap UU may extend between the first set of barbs 3005 and the second set of barbs 3005. In this example, the gap UU may have a length of about 5.0±0.5 mm. An overall length VV of the barbed suture 3000 may be about 75.0±1.0 mm, although the exact length may depend, e.g., on the intended use of the barbed suture 3000. In some aspects, the barbed suture 3000 may also include end portions free from barbs 3005, with a length WW of the end portions being about 20.0±1.0 mm, if included. With reference to FIG. 31, an angle XX between an axis along which a barb 3005 extends and a longitudinal axis, along which the cut, forming the barb 3005 extends, may be about 60°±5°. A height YY of each barb 3005 may be about 0.30±0.03 mm, and a spacing ZZ between a barb 3005 and a cut for an adjacent barb 3005, as shown in FIG. 31, may be about 0.900±0.1 mm. FIG. 32 shows a cross-sectional view, taken along line 32-32 in FIG. 30, of the barbed suture 3000, with one barb 3005 extending in a first direction (e.g., an upward direction in FIG. 32), and another barb 3005 extending in a second direction (e.g., a left direction in FIG. 32). An angle AAA between these two barbs 3005 may be roughly perpendicular to one another, for example, about 90°±5°. FIGS. 33 and 34 are images of another example of a barbed suture 3000, with barbs oriented in two planes.

Although the descriptions of exemplary barbed sutures include ranges of dimensions, e.g., barb height, the length of different portions or of the overall barbed suture, diameter, the relative angles between barbs, etc., it should be understood that the specific dimensions included herein as examples are not limiting, and other dimensions may be used. The dimensions of any individual barbed suture may depend, at least in part, on the materials used, the intended use of the barbed suture, etc.

To optimize the ability of barbs and, therefore, the ability of a barbed suture to engage with and penetrate a tissue or a portion of a tissue, such as an epineurium of a nerve tissue, and therefore, to optimize coaptation strength of tissue being repaired or coapted using the barbed suture, while inhibiting damage to the tissue, certain parameters are considered, including one or more of a number of barbs, barb height, barb spacing (that is, a distance between barbs or a distance between a barb and a cut with which an adjacent barb is formed), plane(s) in which the barbs extend both in terms of direction and number, a shape of a tip of each barb, a sharpness of the tip of each barb, and an angle between a surface of the barb facing the barbed suture and the adjacent surface of the barbed suture (also referred to as a contact angle of the barb). In an embodiment in which barbs are formed by cutting into a surface of a suture, a cutting angle used to form the barbs and a cutting depth (that is, a distance along a length of the surface of the suture which is cut to form the barb) to form each barb may be considered as parameters for optimization as well. In other embodiments in which laser cutting or injection molding is used to form barbs, such as barbs with custom-shaped tips or microhooks, these parameters are also considered.

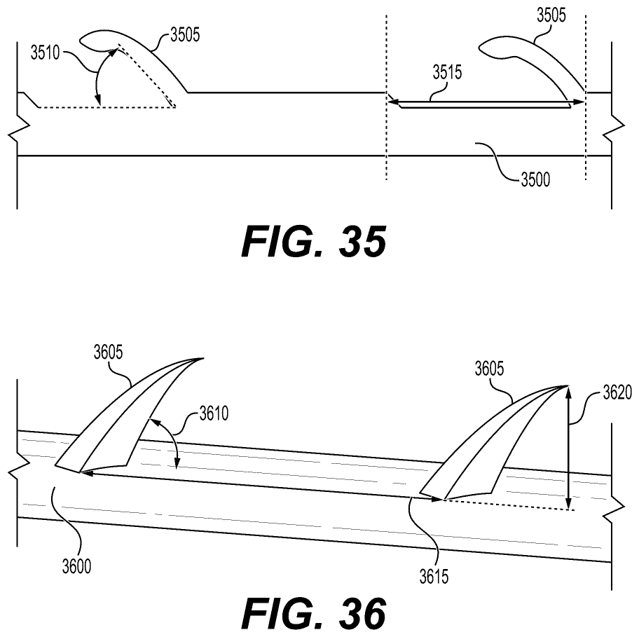

FIG. 35 is a schematic drawing of a barbed suture 3500 with two adjacent barbs 3505, formed using mechanical cutting with a blade (unshown). In some embodiments, laser cutting or compression molding may be used to form the barbs 3505. In particular, FIG. 35 depicts a cutting angle 3510 and a cutting depth 3515 for barbs 3505. As an example, the cutting angle may be about 45°, and the cutting depth may be about 0.75 mm. FIG. 36 is a schematic drawing of a barbed suture 3600, with two adjacent barbs 3605, formed using injection micro molding to form microhooks as barbs 3605. In particular, FIG. 36 depicts an angle of contact 3610, a shape of the barbs, a distance 3615 between adjacent barbs 3605, and a height of a barb 3605 from a surface of the suture 3600.

### III. Method of Forming a Barbed Suture

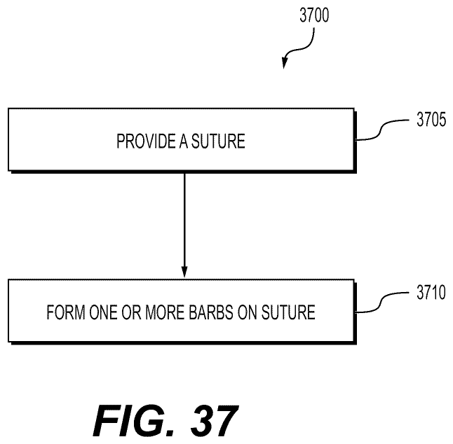

FIG. 37 depicts a flowchart of a method 3700 of forming a barbed suture according to one or more embodiments. The method 3700 may be used to form a suture having at least one barb, according to any one of the embodiments described above. The method 3700 may include a step 3705 of providing a suture, and a step 3710 of forming one or more barbs on the suture. The step 3710 of forming the one or more barbs may be a step of mechanically cutting the suture to form the one or more barbs. The step 3710 may alternatively be a step of laser cutting the suture to form the one or more barbs. Alternatively, the step 3710 may be a step of injection molding one or more barbs by placing the suture in a mold and injecting a material to form the barbs, or by injection molding barbs and affixing them to a suture. In general, the barbs of the barbed sutures described herein may be formed in any suitable manner, such as cutting with a blade or other instrument, laser cutting, injection molding, etc.

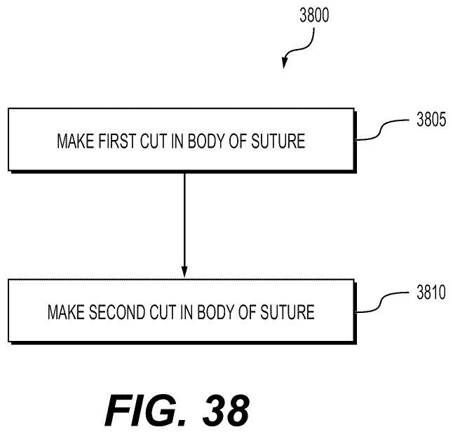

FIG. 38 depicts a flowchart of a method 3800 of forming a barbed suture according to one or more embodiments. The method 3800 may be used to form a suture having at least one bi-directional barb, according to any one of the embodiments described above. The method 3800 includes step 3805 of making a first cut into a body of a suture to form a first projection. The first projection may be oriented in a first direction. The first cut does not extend fully through the body of the suture. The method 3800 also includes a step 3810 of making a second cut into the body of the suture to form a second projection, oriented in a second direction. The second cut does not extend fully through the body of the suture. The first direction is different from the second direction. In particular, the first direction may be substantially opposite to the second direction. Alternatively, the first direction may be at an angle relative to the second direction. The first cut and the second cut may be at the same or different angles, or at the same or different depths into the body of the suture.

Although the method 3800 may include the steps 3805 and 3810 described above, the method 3800 may include additional steps. For example, the method 3800 may include a step of twisting the body of the suture prior to making the first cut into the body. The body of the suture may be twisted a predetermined number of turns (a turn representing 360° of rotation) per a length of the suture. In some aspects, the body of the suture may be twisted up to 10 turns per one linear inch, up to about 5 turns per one linear inch, less than about 1 turn per one linear inch, from about 1 to about 10 turns per one linear inch, from about 4 to about 8 turns per one linear inch, from about 1 to about 5 turns per one linear inch, or from 5 to about 10 turns per one linear inch. As an example, in the embodiment shown in FIG. 21, the body of the suture may be twisted 5.7 turns per one linear inch prior to forming the barbs in the body of the suture. Alternatively, the body of the suture may remain untwisted (i.e., unturned) prior to steps 3805 and 3810. Further, steps 3815 and 3810 may be performed on one side of the body of the suture, so that barbs are formed on only one side of the body of the suture. In some aspects, the steps 3805 and 3810 may be performed repeatedly to form any number of barbs on the suture, in any one of the arrangements described above or shown in FIGS. 3 to 36.

Further, although only two steps and thus two cuts are described in reference to FIG. 38, additional steps may be included in the method 3800 for making additional cuts. For example, an additional step may be included for making a third cut in the body of the suture to form a third projection of the barb oriented in a third direction.

Once formed, the barbed suture may be used alone to affix portions of tissue to one another, or to affix two different tissues to one another, or to affix one or more devices to tissue, for example. An example of this is depicted and described in reference to FIG. 29B. Alternatively, the barbed sutures described herein may be used to form a tissue engaging portion of a larger tissue repair device, such as a mesh, as will be described below in further detail.

### IV. Barbed Tissue Repair Devices

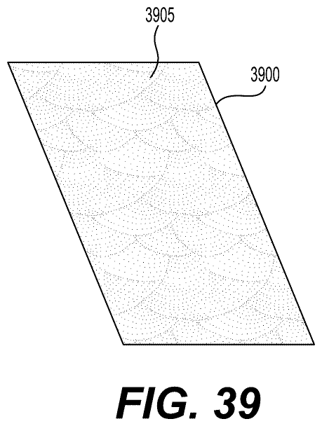

FIGS. 39 to 118 show various embodiments of barbed tissue repair devices and components thereof. Example barbed tissue repair devices may include one or more of barbed sutures, meshes, base membranes, etc., as will be described further herein. In each of these embodiments, the barbed devices may include one or more of the barbed sutures described with respect to FIGS. 1 to 36, or may include alternative barb formations, as will be described below. In some instances, traditional barbs may be used with the barbed devices described with reference to FIGS. 39 to 118.

In addition, in each of these embodiments, components of the barbed devices, e.g., including the barbed sutures, meshes, and base membranes, may be formed of a material, such as a resorbable polymer, or another polymer, e.g., polyester, polydioxanone (e.g., poly-p-dioxanone), poliglecaprone (such as poliglecaprone 25 sold under the tradename Monocryl®), polyglytone (such as polyglytone 6211 sold under the tradename Caprosyn®), catgut, collagen, polyglycolide, polylactide (e.g., poly-L-lactide), polydroxyalkanoates, or one or more other suitable materials, and, in some aspects, one or more of the barbed suture, meshes, or base membranes may be absorbable. In other aspects, the barbed devices may be formed of a biodegradable metal or metal alloys, e.g., biodegradable magnesium, zinc, or iron, or alloys of thereof.

### A. Base Membrane

FIG. 39 is a schematic depiction of a base membrane 3900, which may be used in combination with barbs, meshes, barbed sutures, or barbed devices formed of barbed sutures, to form a tissue repair device according to any of the embodiments described herein. The base membrane 3900 may be referred to as a wrap, a film, or a sheet of film. As an example, the base membrane 3900 may have barbs attached to a surface thereof, or the base membrane 3900 may have one or more barbed sutures or a barbed device attached thereto. As another example, the base membrane 3900 may be applied over or wrapped around a barbed device. Alternatively, the base membrane 3900 may be applied over portions of tissue, and then a barbed device may be applied over or wrapped around the underlying base membrane 3900. In some aspects, ends of the base membrane 3900 may be secured after being applied over or wrapped around barbed sutures or a barbed device, for example, using a suture, and/or an adhesive. In some aspects, use of the base membrane 3900 may provide additional protection for the tissues being repaired, may act as a barrier to prevent the barbed sutures or the barbed device from rubbing on surrounding structures, or may be used to deliver one or more drugs or chemicals to the tissue repair site. If the tissue being repaired is a nerve tissue, then the presence of the base membrane 3900 may inhibit axonal escape as axons regrow from the nerve tissue.

The base membrane 3900 may be formed of any suitable biocompatible material for use in a tissue repair procedure. The base membrane 3900 may be formed of a natural material, a synthetic material, or a combination thereof. The natural material may be extracellular matrix (ECM) material, such as small intestine submucosa (SIS), for example, or more specifically, porcine SIS, amnion-based tissue (e.g., amniotic/chorionic membrane or amnion), dermis, decellularized fascia, reconstituted denatured collagen, elastin, thrombin, fibronectin, starches, poly(amino acid), gelatin, alginate, pectin, fibrin, oxidized cellulose, chitin, chitosan, tropoelastin, hyaluronic acid, fibrin-based materials, collagen-based materials, hyaluronic acid-based materials, glycoprotein-based materials, cellulose-based materials, silks, and combinations thereof. The natural material may be obtained from a human source or an animal source, and may be autogenic, allogenic, or xenogenic with respect to a subject into which a tissue repair device, including the base membrane 3900, is implanted or placed. The synthetic material may be one or more of a resorbable polymer, homopolymers, copolymers, and/or polymeric blends including one or more of the following monomers: glycolide, lactide, caprolactone (including ε-caprolactone), dioxanone (including p-dioxanone), trimethylene carbonate, monomers of cellulose derivatives, and monomers that polymerize to form polyesters. The polymers may include polydioxanone (PDS), polycaprolactone (PCL), polytrimethylene carbonate, polyglycolide (PGL), poly-3-hydroxybutyrate (PHB), poly(3-hydroxybutyrate-co-3-hydroxyvalerate) (PHBV), poly(propylene carbonate) (PPC), poly(butylene succinate) (PBS), poly(propylene fumarate) (PPF), polydroxyalkanoates, or one or more other suitable materials.

The base membrane 3900 may be configured to remodel into host tissue(s) (that is, the tissue(s) on which the tissue repair device having the base membrane 3900 is implanted), and may not leave permanent components, or significant amounts of permanent components, behind. That is, the base membrane 3900 may partially or fully remodel into host tissue(s). The base membrane 3900 may provide circumferential coverage of a tissue repair site, such as a grafting site or a coaptation site, once applied, in an instance in which tubular-shaped tissues are the intended site of application (e.g., nerves, blood vessels, etc.). The base membrane 3900 may protect a covered region of the tissue once applied, and, if used in the case of tissue transection, may protect the coaptation site from an injured tissue bed.

The base membrane 3900 may be formed of a different material than the material that forms the barbs, barbed sutures, or other components of a barbed device, and thus, the base membrane 3900 may have different mechanical properties from said barbs, barbed sutures, or other components of the barbed device. For example, the base membrane 3900 may be more flexible, whereas the barbs, barbed sutures, or barbed device may be relatively more rigid in order to extend into and engage with the tissue. In some aspects, the base membrane 3900 may be configured to resorb at a different speed (e.g., faster or slower) compared to the barbs, the barbed sutures, or the barbed device, or the base membrane 3900 may be designed to resorb at a similar speed compared to the other components.

In some embodiments, the base membrane 3900 may also include a protector or a coating 3905 on one or both surfaces thereof. The coating 3905 may include, for example, one or more of hyaluronic acid, alginate, antibiotics, lubricant, growth-enhancing drugs, growth inhibiting drugs, anti-inflammatory drugs, pain-reducing or anesthetic drugs, or other suitable chemical compounds. Surfaces of the base membrane 3900 may be textured, as shown in FIG. 39, smooth, or a combination thereof. A shape and a size of the base membrane 3900 may depend, at least in part, on a shape and size of a tissue to be repaired using the base membrane 3900, or a shape and size of the barbed device the base membrane 3900 is a component of or is to be used with. In a case in which a barbed device is used with the base membrane 3900, the shape and size of the base membrane 3900 may generally correspond to the shape and the size of the barbed device. However, one or both of the size and shape of the base membrane 3900 may differ from that of the barbed device. For example, in one or more embodiments, the base membrane 3900 may be sized to be larger than the barbed device in width and length, so that the base membrane 3900 covers a non-barbed side of barbed device.

B. Attachment of Barbs, Barbed Sutures, Meshes, etc., to a Base Membrane to form a Barbed Device
a. Attachment Using Adhesive or Glues

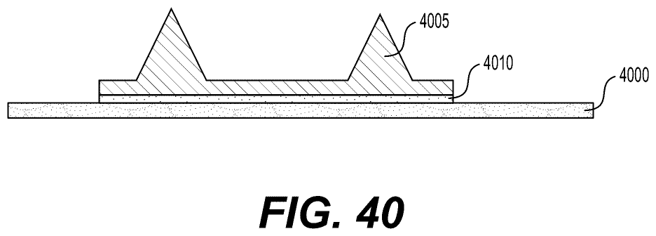

One or more barbs, barbed sutures, or meshes may be attached to the base membrane in any suitable manner. For example, the barbs, barbed sutures, or meshes may be attached to the base membrane using a glue or an adhesive. In some aspects, the glue or adhesive may be applied only between the base membrane and the barbs, barbed sutures, or meshes, with minimal to no spilling of the glue or adhesive elsewhere on the base membrane. The glue or adhesive may be applied by one or more of painting, fluid dispensing, spraying, pipetting, jet printing, or three-dimensional (3D) printing. The glue or adhesive may be first applied to the base membrane, and then the barb, barbed sutures, or meshes may be placed onto the glue on the base membrane. Additionally or alternatively, the glue may be applied to a surface of the barbs, barbed sutures, or meshes configured to attach to the base membrane, and then the barbs, barbed sutures, or meshes may be placed onto the base membrane. In some aspects, the barbs, barbed sutures, or meshes may have a notch or an indent on a bottom surface thereof, and the glue or adhesive may be applied in the notch, and then the barbs, barbed sutures, or meshes may be placed onto the base membrane. The notches may serve to contain the adhesive or glue and to inhibit oozing of the glue or adhesive outside of a footprint of the barbs, barbed sutures, or meshes. In some aspects, individual barbs may be formed and then applied individually, either one by one or a few at a time. In other aspects, barbs may be formed on linear constructs, as shown in FIG. 40, that could be applied to the base membrane, to affix multiple barbs at one time. These barbed constructs may be shorter or less flexible than barbed sutures, but may serve a similar function and may have arrangements of barbs similar to as described above in reference to barbed sutures.

According to some embodiments of the present disclosure, adhesive may be a polymer adhesive, and the polymer adhesive may be biocompatible, i.e., it may be safe to use in humans or animals. The polymer adhesive may be biodegradable, i.e., the polymer may completely or partially degrade over time and may not leave significant permanent material behind. The polymer adhesive may be suitable for clinical adaptation.

Many polymers have a long history of safe use in different clinical products (e.g., sutures), and thus may be appropriate for use in the disclosed embodiments. In some aspects, the polymer adhesive may have a tunable degradation rate, i.e., the polymer composition may be modified to optimize the degradation rate in line with one or more of the tissue type to which the tissue repair device is intended to be applied to, or the intended use of the tissue repair device. In some examples, the polymer adhesive may be used in potential applications for controlled, sustained, and localized delivery of drugs/therapeutics. In such embodiments, the polymer adhesive selected may further be used to act as a drug delivery device.

In some aspects, glues or adhesives may be used with crosslinking agents, such as one or more of polysaccharides, such as methacrylated gelatin (Gel-MA), methacrylated hyaluronic acid (MA-HA), and glycidyl methcrylated hyaluronic acid (GM-HA, which could also function as adhesives.

According to some aspects, the polymer adhesive may be polycaprolactone (PCL), poly(ε-caprolactone-co-lactide) (PLCA), polyhydroxyyalkanoates (PHAs), or polylactic-co-glycolic acid (PLGA). PLGA, a synthetic copolymer of polylactic acid (PLA) and polyglycolic acid (PGA), may be suitable for use in clinical applications due to its biodegradability and biocompatibility. This copolymer is soluble in organic solvents such as acetone, dichloromethane (DCM), chloroform, ethyl acetate, dimethyl sulfoxide (DMSO), and tetrahydrofuran.

Although PLGA is soluble in organic solvents, it biodegrades via hydrolysis of its ester linkages in the presence of water. This biodegradation is a gradual process that may be optimized by adjusting parameters, such as polymer molecular weight and the ratio of lactide to glycolide monomers to achieve the desired degradation rate.

According to some aspects of the present disclosure, a solution of PLGA may be prepared using organic solvents, such as DCM and acetone. After dissolving the PLGA in the solvent, the solution may be used as an adhesive to attach the barbs, barbed sutures, or mesh to the base membrane, for example, an SIS-membrane, forming a tissue repair device. The tissue repair device may air dry to allow the solvent (e.g., DCM or acetone) to evaporate, resulting in the barbs, barbed sutures, or barbed devices being securely glued to the base membrane. In some aspects, the tissue repair device may be left to dry for one or more hours, e.g., approximately 1 hour, approximately 2 hours, approximately 3 hours, approximately 4 hours, from approximately 30 minutes to approximately 4 hours, from approximately 30 minutes to approximately 3 hours, from approximately 30 minutes to approximately 2 hours, from approximately 1 hour to approximately 2 hours, etc.

FIG. 40 shows a detail schematic side view of base membrane 4000, a barbed structure 4005, such as a portion of a barbed suture or a barbed construct, and a layer of a polymer 4010, as a glue or an adhesive, therebetween. The layer of polymer 4010 may be referred to as a polymer interface and may attach the barbed structure 4005 to the base membrane 4000. The base membrane 4000 may be formed of any of the materials described above. The barbed structure 4005 may be a micro-molded barbed construct, as shown in FIG. 40, or it may be a single barb, a barbed suture, or any other barbed component described herein. The polymer 4010 may be any of the polymers described above for use as an adhesive, and as an example, may be formed of PLGA dissolved in a solvent. In such an example, the polymer 4010 may bond the barbed structure 4005 to the base membrane 4000 once the solvent evaporates.

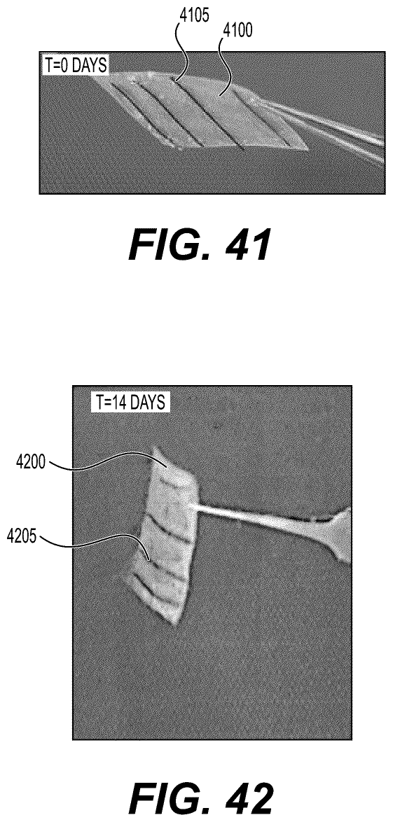

Preliminary testing of the method of making a tissue repair device as described above was performed using a solution of PLGA with organic solvents, such as DCM and acetone. After dissolving the PLGA, the solution was used as an adhesive to attach barbed sutures to a base membrane. The tissue repair device was then left to air dry for approximately two hours. The barbed sutures to the SIS base membrane once the solvent evaporated. The adherence of barbed sutures to the SIS base membrane was tested by submerging the barbed sutures and the SIS base membrane in a saline solution and examining the adhesivity by vigorous movement over time. FIGS. 41 and 42 are images showing the barbed sutures adhered to a base membrane during the testing process. At T=0 days, in FIG. 41, the base membrane 4100 is shown with the side on which the barbed sutures 4105 are adhered facing downward in the direction of gravity. At T=14 days, following 2 weeks of continuous hydration, in FIG. 42, the base membrane 4200 is shown with the side on which the barbed sutures 4205 are adhered facing to the left of the image. These images demonstrate the slow hydrolysis of PLGA, making it a suitable biocompatible adhesive for in vivo applications, such as tissue repair, such as nerve coaptation.

Although PLGA was tested in the initial prototypes, other biodegradable polymers that are soluble in organic solvents and are slowly hydrolyzed in water can be used in place of PLGA as the polymer adhesive. A non-exhaustive list of examples includes: Polycaprolactone (PCL), Poly(lactic acid) (PLA), Polyglycolic acid (PGA), Poly(ε-caprolactone-co-lactide) (PCLA), Polyhydroxyalkanoates (PHAs), etc. In addition, different forms of PLGA can be obtained by altering the ratio of lactide to glycolide used during polymerization. These forms are typically identified by the molar ratio of the monomers used (e.g., PLGA 75:25 denotes a copolymer consisting of 75% lactic acid and 25% glycolic acid). The composition of PLGA monomers determines its solubility rate. Polymers with higher lactide content are usually dissolved in chlorinated solvents, while those with higher glycolide content may be dissolved in fluorinated solvents.

In addition, commonly used kitchen ingredients can also serve as the polymer adhesives. These natural adhesives are easy to make and are non-toxic in nature. For instance, starch paste, gelatin, honey, molasses, rice paste, sugar syrup, etc., may be used.

b. Attachment Using Thermal Bonding

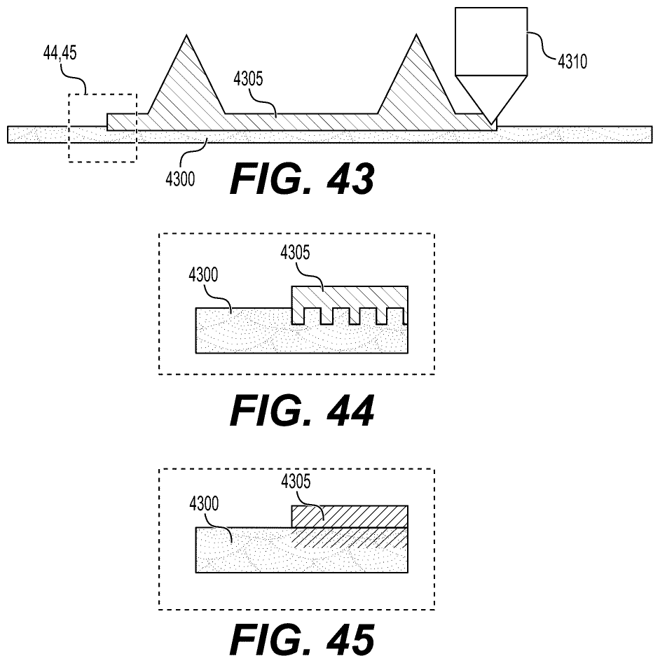

As an alternative, thermal bonding may be used to attach barbs, barbed sutures, or meshes to a base membrane. Specific examples of thermal bonding include spot welding, applying ultrasonic heat, or otherwise applying energy to melt and entangle the barbs, barbed sutures, or meshes, or portions thereof, into the base membrane. That is, the barbed components may be attached using heat, such as by melting the barbed components at specific locations on the base membrane, as the barbed components lay atop the base membrane. FIG. 43 shows a base membrane 4300 with a barbed structure 4305 on top of the base membrane 4300. A spot welding probe 4310 is also shown. FIG. 44 is a detail view of an interface between the thermally bonded barbed structure 4305 and the base membrane 4300. FIG. 45 is a detail view of an alternative interface between the thermally bonded structure 4305 and the base membrane 4300.

c. Attachment Using Weaving or Sewing

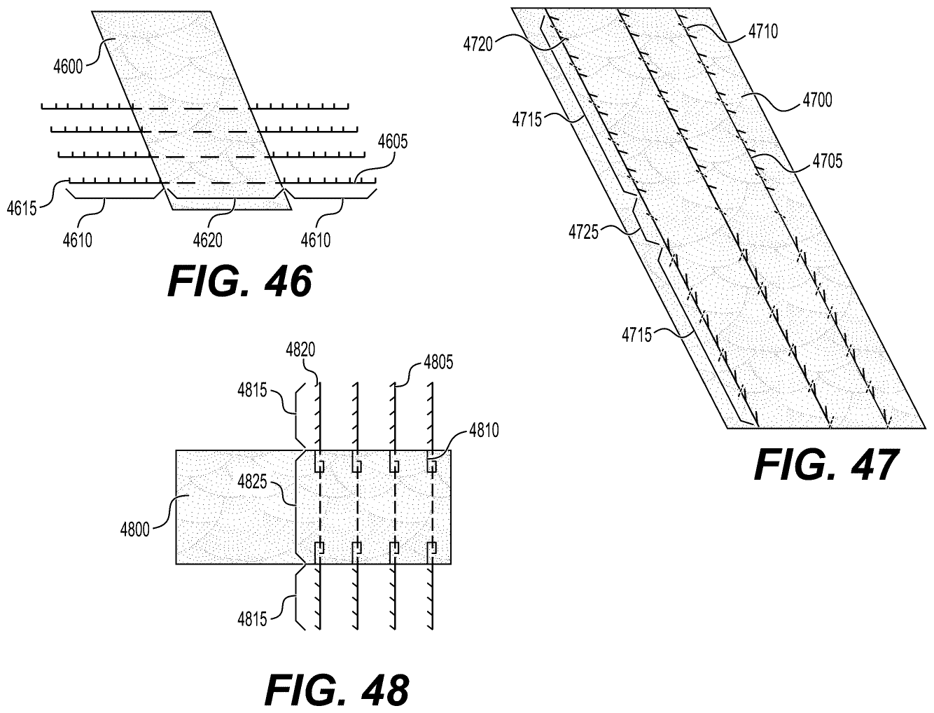

As another alternative, barbed components, such as barbed sutures, may be attached to the base membrane by weaving or sewing the barbed sutures into or through the base membrane. FIG. 46 shows one embodiment in which barbed sutures 4605 are woven through a base membrane 4600. One or more barbed suture 4605 may have barbed portions 4610, having barbs 4615 thereon, and a non-barbed portion 4620 in a central region of the barbed suture 4605. The non-barbed portion 4620 may be woven through the base membrane, with the barbed portions 4610 extending out from sides of the base membrane 4600, as shown. Alternatively, although not shown, barbs 4615 may extend along the entire length of the barbed suture 4605, and the barbed portion 4620 may be woven through the base membrane 4600. The weaving of one or more barbed sutures 4605 in this manner may form a permanent attachment between the base membrane 4600 and the barbed sutures 4605.

FIG. 47 shows another embodiment in which barbed sutures 4705 are sewn in place on a base membrane 4700. In one aspect, the barbed sutures 4705 may be sewn by passing through holes 4710 in the base membrane 4700. The holes 4710 may be evenly spaced along the base membrane 4700, as shown. In other embodiments, the holes 4710 may be non-evenly spaced along the base membrane 4700. As with the embodiment shown in FIG. 46, each barbed suture 4705 may have barbed portions 4715, having barbs 4720 thereon, and a non-barbed portion 4725 in a center of the barbed suture 4705, or may have barbs 4720 extending along the entire length. In this embodiment, both the barbed portions 4715 and the non-barbed portions 4725 of the barbed sutures 4705 are on the base membrane 4700. The sewing of the barbed sutures 4705 in this manner may form a permanent attachment between the base membrane 4700 and the barbed sutures 4705.

In other aspects, holes 4710 may be replaced with stitches 4710, and one or more barbed sutures 4705 may be placed on a surface of the base membrane 4700 and then stitched in place on top of a surface of the base membrane 4700 such that the barbs 4720 extend away from the base membrane.

In some embodiments, the barbed sutures may be attached to the base membrane by forming cuts or notches, such as J-shaped cuts or S-shaped cuts, and inserting or hooking barbed sutures into the cuts. FIG. 48 depicts a base membrane 4800 with barbed sutures 4805 attached using cuts or hooks 4810 in the base membrane 4800. The cuts 4810 may be located along one or more edges of the base membrane 4800, such that the barbed sutures may be inserted or hooked through the cuts 4810 and thereby attached to the base membrane 4800. In some aspects, each cut 4810 may be formed on an opposite edge to another cut 4810, as shown. Each barbed suture 4805 may have barbed portions 4815, having barbs 4820, and a non-barbed portion 4825 in a central region of the barbed suture. The barbed suture 4805 may be hooked into the base membrane 4800 such that the non-barbed portion 4825 overlaps with the base membrane 4800, and the barbed portions 4815 extend out from sides of the base membrane 4800, as shown. In other aspects, barbs 4820 may extend an entire length of barbed suture 4805 and may overlay the base membrane 4800. The hooking of the barbed sutures 4805 in this manner may form a non-permanent attachment between the base membrane 4800 and the barbed sutures 4805. That is, with this configuration, the barbed sutures 4805 may be removably attached to the base membrane 4800.

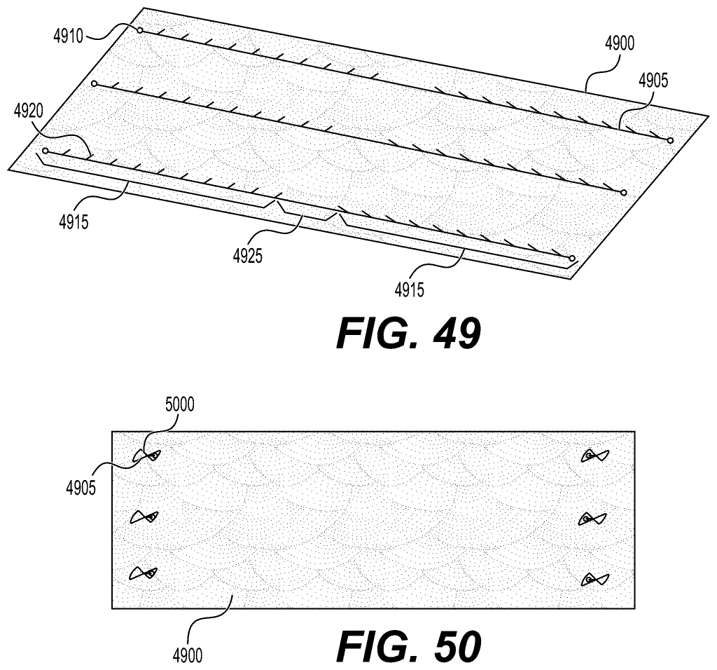

In other embodiments, the barbed sutures may be attached to the base membrane by forming small holes or cuts in the base membrane, threading the barbed sutures through the holes, and tying knots in the ends of the barbed sutures once threaded through the holes. FIG. 49 depicts a base membrane 4900 having holes 4910, with barbed sutures 4905 attached by being threaded through the holes 4910 in the base membrane 4900. The holes 4910 may be located along one or more edges of the base membrane 4900, such that the barbed sutures 4905 may be inserted through the holes 4910. In some aspects, each hole 4910 may be located on opposite edges relative to another hole 4910, as shown. Each barbed suture 4905 may have barbed portions 4915, having barbs 4920, and a non-barbed portion 4925 in a central region of the barbed suture. The barbed suture 4905 may be attached to the base membrane 4900 such that both the barbed portions 4915 and the non-barbed portion 4925 overlap with the base membrane 4900. In other aspects, barbs 4920 may extend an entire length of barbed suture 4905 and may overlay the base membrane 4900.

FIG. 50 shows a back side of the base membrane 4900 and the ends 5000 of barbed sutures 4905 shown in FIG. 49 passed through the holes 4910 and tied in knots. The tying of the ends 5000 of the barbed sutures 4905 in knots may form a permanent attachment between the base membrane 4900 and the barbed sutures 4905. In some embodiments, a glue or an adhesive, including any of those described above, may be used between the barbed sutures 4905 and the base membrane 4900 to provide additional support and attachment, either instead of, or in addition to, the use of knots to secure the ends 5000 of the barbed sutures 4905. In some aspects, the ends 5000 may be tied in knots, with or without adhesive, too, and the middle portion of the barbed sutures 4905 may be secured in place with adhesive or stitches.

d. Attachment using Slits Formed in the Barbed Sutures

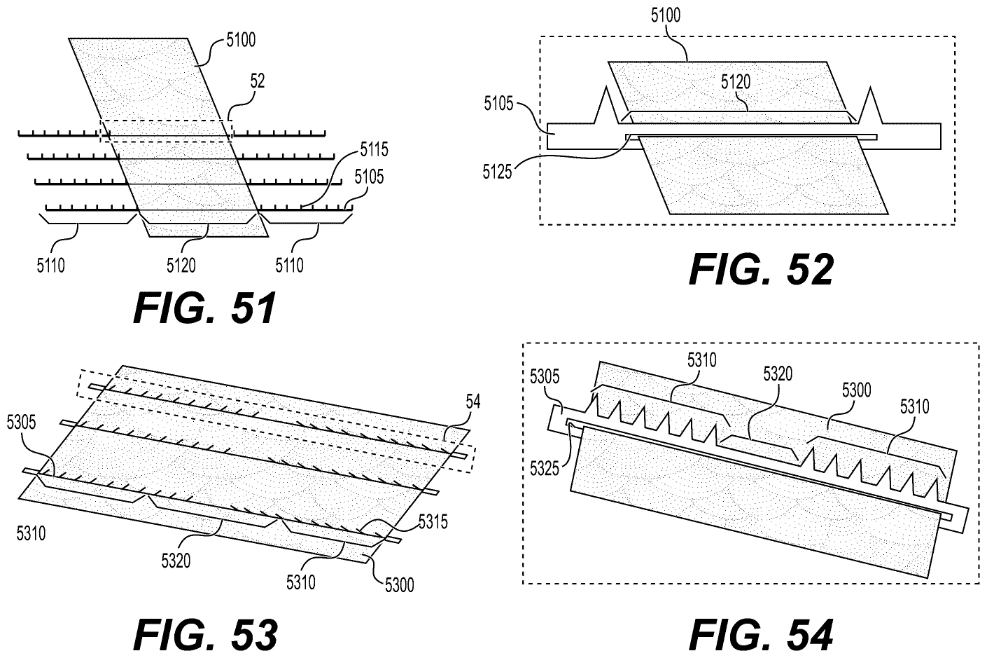

As another example, the base membrane may be attached to the barbed sutures by inserting the base membrane into a thin slit or cut that extends through the barbed sutures. FIG. 51 depicts barbed sutures 5105 attached to a base membrane 5100 in this manner. Each barbed suture 5105 may have barbed portions 5110, having barbs 5115, and a non-barbed portion 5120 in a central region of the barbed suture 5105. FIG. 52 is a detail view of a slit 5125 in the non-barbed portion 5120 of the barbed suture 5105 with the base membrane 5100 passing through the slit 5125. The slit 5125 may have a length and a width that is sufficient for the base membrane 5100 to pass through. With the base membrane 5100 passing through the non-barbed portions 5120 of the barbed sutures 5105, the base membrane 5100 overlaps with the non-barbed portions 5120, and the barbed portions 5110 extend out from sides of the base membrane 5100, as shown in FIG. 51. In other aspects, barbs 5115 may extend along an entire length of barbed suture 5105, and a slit 5125 may thus be formed through a barbed region of barbed suture 5105 such that barbs 5115 overlap with the base membrane 5100. In some aspects, glue or adhesive may be used to maintain the base membrane 5100 within the slit 5125, while in other aspects, no adhesive or glue may be used.

FIG. 53 depicts another embodiment in which barbed sutures 5305 are attached to a base membrane 5300 in this manner. Each barbed suture 5305 may have barbed portions 5310, having barbs 5315, and a non-barbed portion 5320 in a central region of the barbed suture 5305. FIG. 54 is a detail view of a slit 5325 passing through both the barbed portions 5310 and the non-barbed portion 5320 of the barbed suture 5305 with the base membrane 5300 passing through the slit 5325. The slit 5325 may have a length and a width that is sufficient for the base membrane 5300 to pass through. With the base membrane 5300 passing through the barbed portions 5310 and the non-barbed portions 5320 of the barbed sutures 5305, the base membrane 5300 overlaps with both the barbed portions 5310 and the non-barbed portions 5320, as shown in FIG. 53. Alternatively, as described above, barbs 5315 may extend along an entire length of barbed sutures 5305, or barbs 5315 may not overlap with the base membrane 5300. In some aspects, glue or adhesive may be used to maintain the base membrane 5300 within the slit 5325, while in other aspects, no adhesive or glue may be used.

As still another example, rather than using barbed sutures having a slit formed within the suture body, two barbed sutures, or a barbed suture and a non-barbed suture, may be attached together, leaving a gap between the two sutures. This arrangement may form a slit-like opening through which the base membrane may be passed in a similar manner as the slits described above in reference to FIGS. 51-54. In one example, the base membrane may be attached to the barbed sutures by attaching two barbed sutures together with glue on their ends and inserting the base membrane through an opening formed between the attached barbed sutures.

e. Attachment Using Sandwiching of Base Membranes

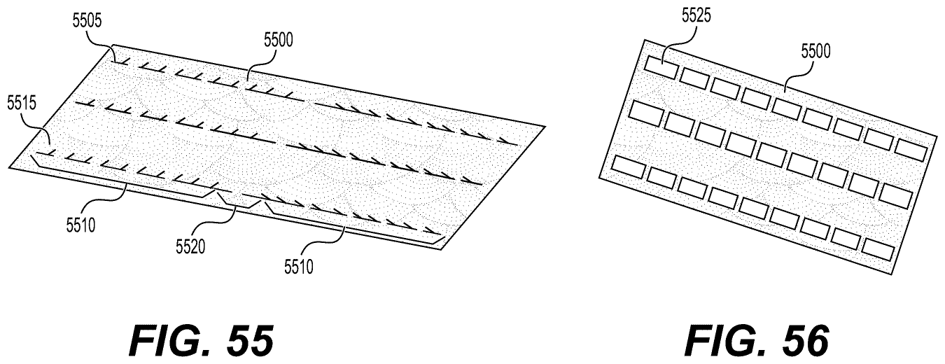

In another example, the base membrane may be attached to the barbed sutures or other barbed components by sandwiching barbed sutures or other barbed components between multiple layers of base membrane material. FIG. 55 depicts barbed sutures 5505 attached to layers of base membrane 5500 in this manner. Each barbed suture 5505 may have barbed portions 5510, having barbs 5515, and a non-barbed portion 5520 in a central region of the barbed suture 5505. Alternatively, barbs 5515 may extend an entire length of barbed suture 5505. FIG. 56 depicts a layer of base membrane 5500 having openings 5525 extending through the base membrane 5500. The openings 5525 may be arranged on the base membrane 5500 in a pattern and may be spaced from one another in an arrangement that compliments the arrangement of the barbed sutures 5505 on the other layer of base membrane 5500. Once barbed sutures 5505 are placed on a first or base layer of base membrane 5500, a second or top layer of base membrane 5500 may be placed on top of the barbed sutures, sandwiching the barbed sutures 5505 between the layers of base membrane 5500 while allowing barbs 5515 to extend through the openings 5525 formed in the top layer of base membrane 5500, as shown in FIGS. 55 and 56. The layers of base membrane 5500 may be attached or secured together using any one of the glues or adhesives described above, or in any other suitable manner. For example, they may be sewn together, welded, or dehydrated to adhere the layers of base membrane to one another.

f. Attachment Using Etching

As still a further example, the barbed sutures may be attached to the base membrane using etching, in which the barbed sutures are arranged in a desired orientation in a container, and a material that forms the base membrane (e.g., a resorbable polymer, or any of the other materials listed above as being used to form the base membrane) is cast around the barbed sutures arranged in the container, such that the barbed sutures are completely covered in the material. Then, the material may be selectively etched to expose the barbed sutures, on at least one end, and another end of the material may be sliced to cut a thin film, thereby forming a tissue repair device, with the barbed sutures embedded within the base membrane.

### C. Arrangement of Barbed Sutures on Base Membrane

In embodiments in which barbed sutures or other barbed components are attached to one or more layers of base membrane, factors including the size of the barbed components in terms of length and diameter, the spacing of barbed components relative to each other on the base membrane, the location of the barbed components on the base membrane, a length and a width of the base membrane, and a number of barbed components on the base membrane may be varied depending on the intended use and function of the tissue repair device. One or more of these factors may depend, at least in part, on the type or size of tissue to which the tissue repair device is to be applied, or the procedure for which the tissue repair device is to be used.

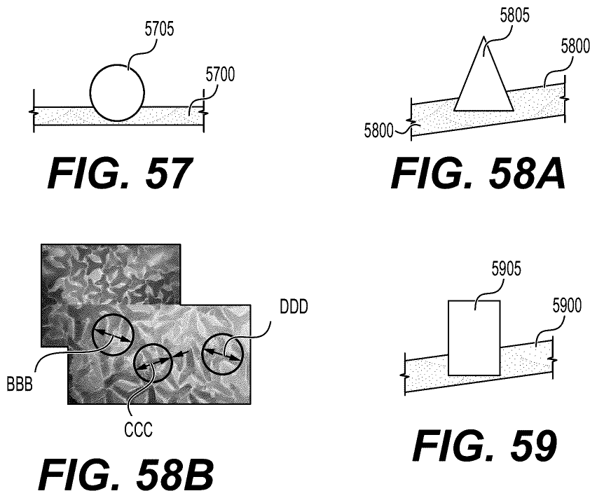

FIG. 57 is a schematic cross-sectional view of a barbed component, in this case, a barbed suture 5705, on a base membrane 5700. The barbed suture 5705 has a circular cross-sectional shape, as shown. Although a circular cross-sectional shape may be suitable for use with the tissue repair devices described herein, a circular cross-sectional shape may result in tipping or rolling of the barbed suture 5705 on the base membrane 5700. In order to engage the tissue to which the tissue repair device is to be applied, ideally the barbs of the barbed suture 5705 face out away from the base membrane 5700, e.g., approximately perpendicular to the base membrane 5700 or at an angle. However, rolling or tipping of the barbed suture 5705 may result in barbs on the barbed suture 5705 not facing away from the base membrane 5700, e.g., facing parallel to the base membrane 5700, towards the base membrane 5700, or at a lesser angle than otherwise intended.

To inhibit rolling or tipping of a barbed component on the base membrane 5700, the barbed component may be formed having a substantially flat side or surface along which it is attached to the base membrane. A flat surface may also facilitate attachment of the barbed suture 5805 to the base membrane 5800, by inhibiting movement rolling during the attachment process (such as those described above). For example, a flat surface may make it easier to sew or weave the barbed suture 5805 in place, by providing a flat surface for applying glue, etc. For example, FIG. 58A is a schematic cross-sectional view of a barbed suture 5805 on a base membrane 5800. The barbed suture 5805 has a triangular cross-sectional shape, as shown, which provides a flat surface for to prevent tipping or rolling of the barbed suture 5805. Similar to barbed suture 5805, other triangular-shaped barbed components, such as triangular shaped barbs or barbed constructs, may be used.

FIG. 58B is an image of cross-sections of multi-lobed, e.g., trilobal, barbed sutures. Diameters BBB, CCC, and DDD of circles defined by lobes of the multi-lobed barbed sutures shown in FIG. 58B may be, for example, about 148.7 μm, about 142.0 μm, or about 150.8 μm. Although the trilobal barbed sutures may not have a flat surface, the lobes may similarly inhibit tipping or rolling of a barbed suture. In some aspects, the multi-lobed, e.g., trilobal, cross-sectional shape may also facilitate attachment of the barbed suture to the base membrane as described above, or may additional provide an indent or ridge into which glue or adhesive may be applied. Other multi-lobed, e.g., trilobal, barbed components, such as trilobal shaped barbs or barbed components, may be used.

FIG. 59 is a schematic end view of a barbed suture 5905 on a base membrane 5900. The barbed suture 5905 has a rectangular cross-sectional shape, as shown, which also provides a flat surface for ease of attachment to the base membrane and which serves to inhibit tipping or rolling of the barbed suture 5905. The barbed sutures shown in FIGS. 58A, 58B, and 59 further may provide relatively sharp barbs, e.g., when the barbs are formed using cutting for example, as compared to barbs formed on a circular-shaped barbed suture. A cut formed along a corner of a triangular suture may be more pointed than a cut formed along a portion of a rounded suture. Similarly, other trilobal barbed components, such as triangular shaped barbs or barbed constructs, may be used.

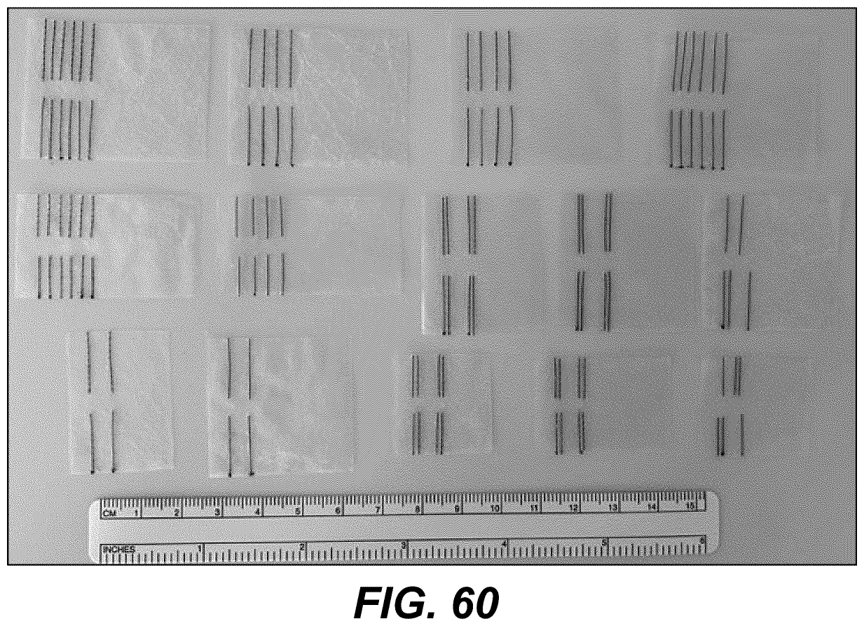

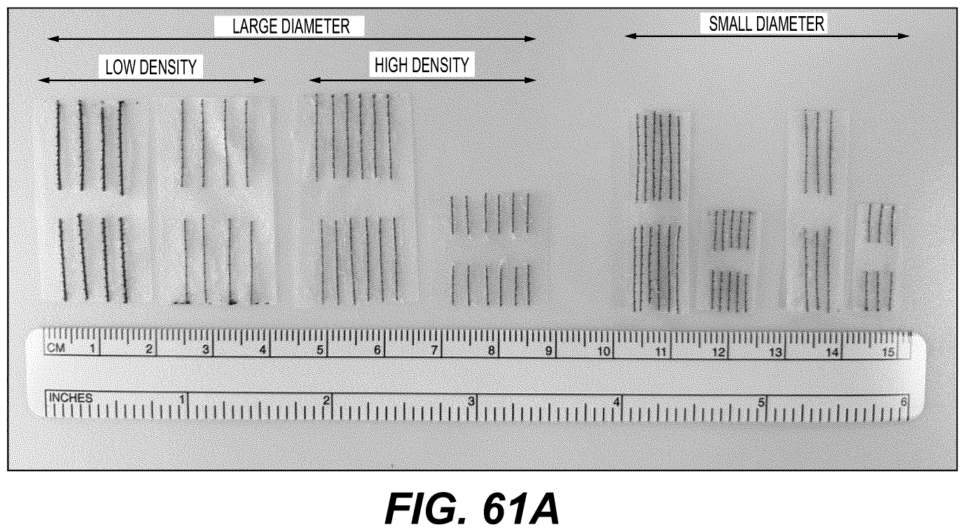

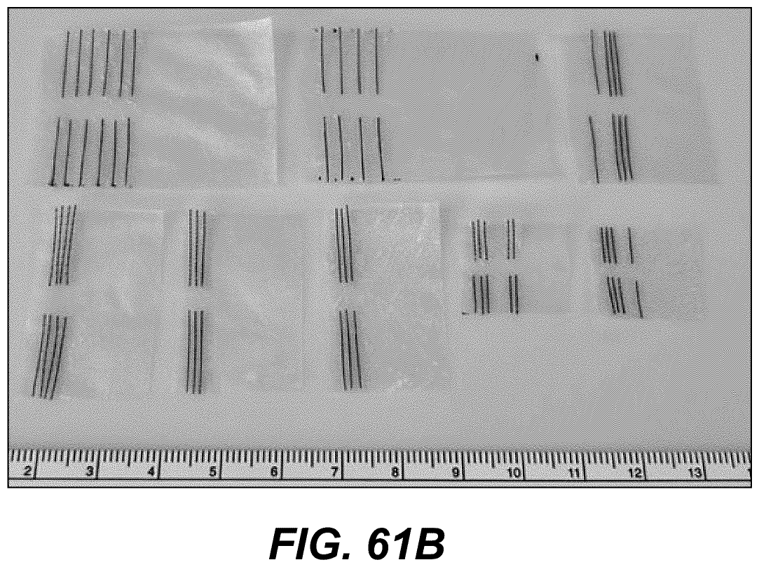

FIGS. 60, 61A, and 61B are images of different examples of tissue repair devices having barbed sutures attached to base membranes. A measuring ruler is included in these figures for reference to demonstrate spacing that is relatively closer together or spaced apart. The specific measurements shown in respect to these prototypes, however, should not be interpreted as limiting, and any suitable spacing may be used, depending on the intended use of the tissue repair device, manufacturing constraints, etc. In particular, as denoted in regards to the image of FIG. 61A, tissue repair devices having barbed sutures with larger diameters are depicted on the left side of the figure, and tissue repair devices having smaller diameters are depicted on the right side of the figure. Low density indicates barbed sutures being arranged with a relatively larger spacing therebetween, and high density indicates barbed sutures being arranged with a relatively smaller spacing therebetween. Some of the tissue repair devices shown have barbed sutures with relatively short lengths, which may be useful, for example, in tight anatomical spaces. In some embodiments, the barbed sutures extend longitudinally along a length of the tissue repair device, while in other embodiments, the barbed sutures extend substantially perpendicular to the length of the tissue repair device. Further, barbed sutures may be arranged to provide a space in a central region of a base membrane that is free from barbs or barbed sutures, as shown in the examples in FIGS. 60, 61A, and 61B. This may be achieved by arranging the barbed sutures in two columns, with each column including aligned barbed sutures. Alternatively, this may be achieved by using barbed sutures that include a non-barbed portion in a central region.

The examples shown in FIGS. 60, 61A, and 61B may have a single layer of base membrane, or more than one layer, such as two layers, three layers, four, or five layers or more.

As depicted in the figures, barbed components, such as barbed sutures, may be spaced along the entire base membrane, as in FIG. 61A, or may be clustered in one or more regions of the base membrane, as in FIGS. 60 and 61B. In some aspects, the positioning of barbed components on the base membrane may effect, e.g., how the tissue repair device is applied to the underlying tissue during use, as is described further below.

In embodiments in which barbed components, such as barbed sutures, are included along the entire base membrane, the tissue repair device may engage the underlying tissue wherever it is applied to the tissue. For example, if the underlying tissue is tubular, as in the case with a nerve, then the first portion of the tissue repair device may engage the underlying tissue, and subsequent portions of the tissue repair device may also engage the underlying tissue. The last portion of the tissue repair device applied to the tissue may be held in place by the barbed components located there, or may additionally be held in place by a suture, adhesive, a gel, or other securing mechanism, or by capillary action. If the underlying tissue is tubular, as in the case with a nerve, then the first portion of the tissue repair device may engage the underlying tissue, and subsequent portions of the tissue repair device may not engage the underlying tissue.

In embodiments in which barbed components, such as barbed sutures, are positioned along less than the entire base membrane, e.g., along about ⅔ of the base membrane, along about ¾ of the base membrane, along about half of the base membrane, along less than half of the base membrane, or along about ⅓ of the base membrane, along about ¼ of the base membrane, etc., only the first portion or only the last portion of the base membrane may engage the underlying tissue. The last portion of the tissue repair device applied to the tissue may be held in place by a suture, adhesive, a gel, or other securing mechanism, or by capillary action.

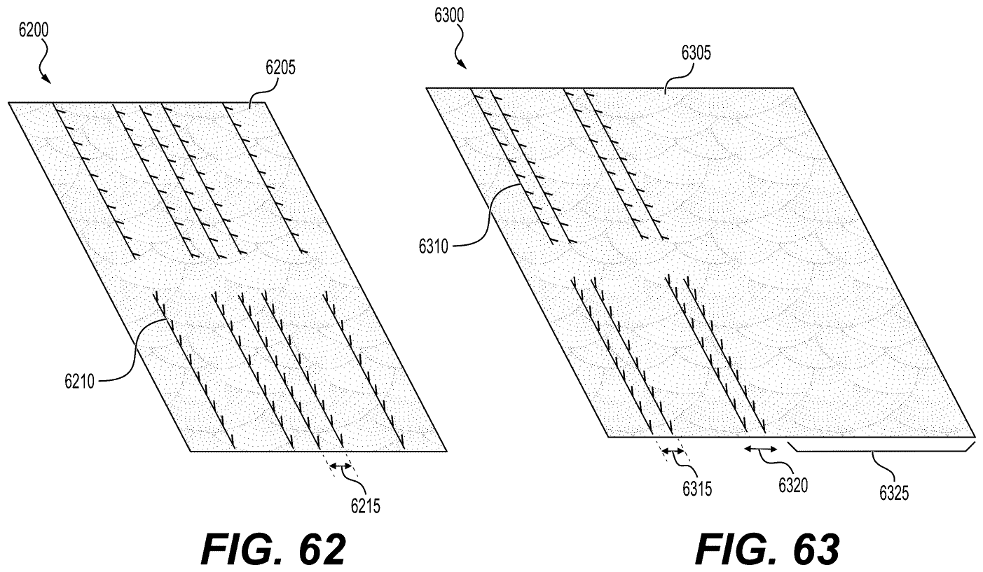

In some aspects, tissue repair devices may have what will be referred to as one or more ‘landing zones,’ with landing zones referring to an area of the base membrane on which tissue is to be positioned. FIG. 62 depicts a tissue repair device 6200 having a base membrane 6205 and barbed sutures 6210, with a landing zone 6215 on which tissue, e.g., two tissue ends, are to be placed. As shown, there may be a relatively higher density of barbed sutures within the landing zone, with six barbed sutures being located in the landing zone, in sets of three, at a relatively small spacing from each other, and four barbed sutures outside of the landing zone, at a relatively larger spacing from adjacent barbed sutures. FIG. 63 shows another tissue repair device 6300 having a base membrane 6305 and barbed sutures 6310, with a landing zone 6315 along a left side of the base membrane 6305. The tissue repair device 6300 may also have additional barbed sutures in an area 6320 adjacent to the landing zone, to provide for engagement of tissue at about 180° from the landing zone, once tissue is wrapped in the tissue repair device 6300, in the case of use with a tubular tissue. A flap 6325, or an area of the base membrane 6305 free from barbed sutures, may also be provided for ease of wrapping the tissue and closing the tissue repair device 6300 around the tissue. Excess material may be trimmed from the flap 6325 after wrapping of tissue. Within the landing zones of the embodiments shown in FIGS. 62 and 63, barbs may be angled to promote engagement with tissue. In one example embodiment, the barbs may be at an angle of about 45° relative to a surface of the base membrane, although angles of about 20° to about 90° may be suitable.

Clustering barbed components, such as barbed sutures, in a landing zone as is shown in some of the examples in FIG. 61B and in the embodiments of FIGS. 62 and 63, may facilitate the attachment of the tissue repair device to the tissue on which it will be positioned. For example, when applying tissue repair devices to small or thin tissues, such as nerves, clustering barbed sutures may make it easier for a user to position the tissue repair device and the tissue relative to one another so that the barbs engage the tissue. One single line of barbed sutures may be harder to position relative to a nerve, or vice versa, but having a higher concentration of barbed sutures in a landing zone may increase the likelihood that at least some of the barbs will engage the tissue.

### D. Barbed Mesh

FIG. 64A is a schematic view of a tissue repair device 6400, according to one embodiment, having a barbed mesh 6402, as a barbed device. The barbed mesh 6402 may be formed of a plurality of barbed sutures 6405, for example, a plurality of longitudinal and lateral barbed sutures 6405 arranged in a grid pattern. Each barbed suture 6405 may include one or more barbs 6410 extending from a surface of the barbed suture 6405 configured to contact with a tissue. In the embodiment shown in FIG. 64A, each barbed suture 6405 may include a plurality of barbs 6410, along a length of the barbed suture 6405. The barbs 6410 may be evenly spaced along each barbed suture 6405, or the barbs 6410 may be arranged at varying spacing or randomly.

Dimensions of the barbed sutures 6405 and the barbed mesh 6402, may depend, e.g., upon one or both of the type of barb (i.e. the directional arrangement of the barbs, the material of the barbs, the shape of the barbs, the size of the barbs, etc.) used, the intended use (e.g., tissue type or procedure type) of the barbed mesh 6402, and the targeted anatomical region. Further, the type of barb used may at least in part determine the number of barbs necessary to form adequate attachment of the barbed mesh 6402 to tissue. The targeted anatomical region may at least in part determine the total area of coverage needed and the force of attachment needed for the barbed mesh 6402 to secure to the tissue surface. A spacing between barbed sutures 6405 may be in a range of about 100 microns (which may be, for example, a width of a 10-0 suture) to about 2 cm. A range of a length and/or a width of the barbed mesh 6402 may, in turn, be from about 300 microns to about 10 cm.

While FIG. 64A depicts barbs on barbed sutures 6405 extending in both longitudinal and lateral directions, it may be that barbed sutures 6405 are used in only one of the longitudinal or lateral directions, and sutures without barbs may be used in the other direction. In other aspects, not all of the sutures extending in a given direction may have barbs. For example, only sutures extending along a portion (e.g., a half of a tissue repair device 6400, or along one or more edges of a tissue repair device 6400) may include barbs, and sutures used in the mesh in other locations may be free of barbs.

Each barbed suture 6405 may be woven together or overlaid with the other barbed sutures 6405, or the barbed sutures 6405 may be connected to one another where two or more barbed sutures 6405 intersect, or may be one, uniform mesh instead of woven or overlaid barbed sutures 6405. In some aspects, the barbed mesh 6402 may be 3D printed, woven from barbed sutures 6405, or formed in any other suitable manner.

Although FIG. 64A shows uni-directional barbs 6410, in one or more embodiments, the barbs 6410 may be multi-directional. As one specific example, barbs 6410 on one half of the barbed mesh 6402 may point or extend in a first direction, while barbs 6410 on another half of the barbed mesh 6402 may point or extend in a second direction. Further, the barbs 6410 may be multi-directional in that each barb 6410 extends towards or away from a point or a line along the barbed mesh 6402 (e.g., a lateral line, a longitudinal line, or an angled line along the barbed mesh 6402, including, more specifically, a center line between the two halves of the barbed mesh 6402). Specifically, in one or more embodiments, barbs 6410 on two “sides” of the barbed mesh 6402, relative to a central line across the barbed mesh 6402, may point or extend towards or away from the central line. And, similarly, in one or more embodiments, barbs 6410 may point or extend towards or away from a single point, which may be any point on the barbed mesh 6402, including a central point (that is, a center of the barbed mesh 6402).

Other directional patterns or arrangements of barbs 6410 may also be used, and such arrangements may be configured for attachment of the barbed mesh 6402 to a particular type of surface of a tissue, such as a curved surface, including a convex curved surface, a concave curved surface, a planar or flat surface, a tubular surface, and combinations thereof. In some embodiments, the directional pattern of the barbs 6410 may be random, such that the barbs 6410 of the barbed mesh 6402 randomly entangle the barbed mesh 6402 with any surface topography of a tissue, to thereby hold and fix the tissue.

In a directional arrangement in which the barbs 6410 point or extend towards a central point, the barbed mesh 6402 may be configured for attachment to a spheroid shaped surface of a tissue. In a directional arrangement in which barbs 6410 point or extend away from a central point, the barbs 6410 may be used to attach the barbed mesh 6402 to a concave curved surface (e.g., an inverse spheroid-shaped tissue surface). In a directional arrangement in which barbs 6410 point or extend towards a line on the barbed mesh 6402, such that the barbs 6410 are parallel to each other and converge towards the line, the barbed mesh 6402 may be configured for attachment to cylindrical shaped tissue surfaces, with the line toward which the barbs 6410 face being determined based on the targeted anatomical region or tissue.

During use, as will be described further below, the tissue repair device 6400 may be wrapped around a tissue or placed on a tissue, or to multiple portions of a tissue, e.g., a transected nerve. The barbs 6410 may hold the barbed mesh 6402 on the tissue and/or may hold tissues in place relative to one another. The holding or fixation of tissue, including nerve tissue or tissue types other than nerve tissue, may be achieved by the uni-directional or multi-directional arrangement of barbs 6410, as described above. The tissue repair device 6400 may be used by itself, or may be overlaid with a base membrane, as will be described further below.

In some aspects, the barbed mesh 6402 may be in the form of a sheet, while in other aspects, the barbed mesh 6402 may be pre-formed into a generally cylindrical shape, as shown in the images of FIGS. 64B and 64C. That is, the barbed mesh 6402 may be rolled into a cylindrical shape. Then, tissue ends may be inserted into ends of the rolled barbed mesh 6402, or the barbed mesh 6402 may be unfurled or flexed outward and positioned around the tissue ends, to hold and fix the tissue ends in place relative to one another. Although transected tissue ends are discussed in reference to this embodiment, the tissue repair devices of this embodiment may be applied to injured tissues of any type, whether fully transected or not, e.g., for protection or to allow the underlying tissue to heal, or simply to secure tissue for any other suitable reason, unrelated to healing. For example, instead of two tissue ends being brought together, embodiments of the disclosure may wrap around or otherwise cover a portion of tissue that is intact, to promote repair or to otherwise protect the underlying tissue.

a. Use of Barbed Mesh with or without a Base Membrane

FIG. 65 is a schematic view of use of the tissue repair device 6400 shown in FIG. 64A, during a tissue repair process, according to one embodiment. In particular, FIG. 65 shows the barbed mesh 6402, including barbed sutures 6405 and barbs 6410, and two transected tissue ends 6500a and 6500b. The two transected tissue ends 6500a and 6500b may be placed on the barbed mesh 6402, so that a portion of the barbed mesh 6402 is positioned on one of the transected tissue ends 6500a or 6500b, and another portion of the barbed mesh 6402 is positioned on the other one of the transected tissue ends 6500a or 6500b. The transected tissue ends 6500a and 6500b may be arranged so that the end regions of the transected tissue ends 6500a and 6500b are positioned on the barbed mesh 6402. The transected tissue ends 6500a and 6500b may be positioned relative to one another so that a gap G₆₅ formed between the transected tissue ends 6500a and 6500b may be approximately 0 mm or may be less than about 5 mm. The transected tissue ends 6500a and 6500b may be touching or almost touching. In other aspects, gap G₆₅ may be greater than approximately 0 mm, for example, up to about 10 mm or up to about 5 mm. For example, the gap may be about 0 mm to about 10 mm, about 0 mm to about 8 mm, about 1 mm to about 10 mm, about 1 mm to about 8 mm, about 2 mm to about 8 mm, about 3 mm to about 7 mm, about 3 mm, about 4 mm, about 5 mm, about 6 mm, about 7 mm, etc. Once the tissue ends are positioned relative to one another, the barbed mesh 6402 may be wrapped around the surface of each of the transected tissue ends 6500a and 6500b.

Wrapping the tissue repair device 6400 may include physically moving (e.g., rolling, twisting, or pulling) the barbed mesh 6402 along the surface of the tissue and laying it on the external surface of the transected tissue ends 6500a and 6500b, rolling the transected tissue ends 6500a and 6500b on the barbed mesh 6402, or a combination thereof. In some aspects, ends of the barbed mesh 6402 may be secured after wrapping, for example, using a suture, an adhesive, a gel, or capillary action. In other aspects, the barbs 6410 may be sufficient to hold the tissue ends 6500a and 6500b in place relative to the barbed mesh 6402. By this configuration, the barbed mesh 6402 secures the transected tissue ends 6500a and 6500b together so that a tissue repair process may occur. More specifically, barbs 6410 on the barbed sutures 6405 of the barbed mesh 6402 may protrude into or otherwise engage with the outer surface of each of the transected tissue ends 6500a and 6500b, and the barbs 6410, together with the barbed sutures 6405 of the barbed mesh 6402, may inhibit the transected tissue ends 6500a and 6500b from moving apart.

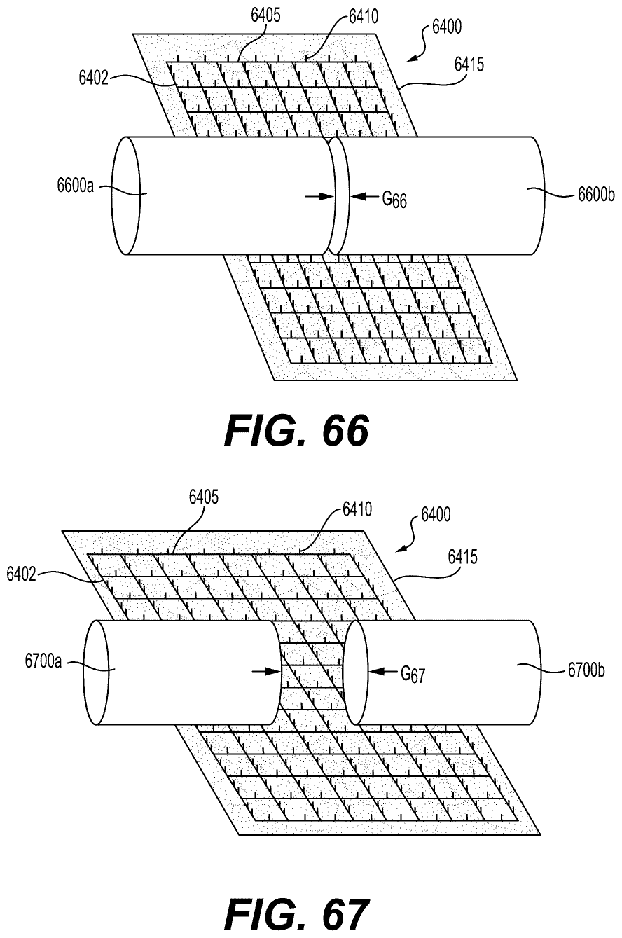

FIG. 66 is a schematic view of a configuration of the tissue repair device 6400, including a base membrane 6415, having one or more of the properties of the base membrane 3900, shown in FIG. 39, and the barbed mesh 6402, shown in FIG. 64A, during a tissue repair process, according to another embodiment. In particular, FIG. 66 shows the tissue repair device 6400, including the barbed mesh 6402 and the base membrane 6415, and two transected tissue ends 6600a and 6600b. The base membrane may be separate from and aligned with the barbed mesh 6402, or may be attached to the barbed mesh 6402. The base membrane 6415 and the barbed mesh 6402, if separate from one another as opposed to attached together, may be packaged together and sold as a pair, or may be packaged and sold individually.

Similar to the configuration shown in FIG. 65, in this configuration, two transected tissue ends 6600a and 6600b may be placed on the barbed mesh 6402, but in this embodiment, the barbed mesh 6402 is layered with the base membrane 6415, so that the barbed mesh 6402 is positioned between the base membrane 6415 and the transected tissue ends 6600a and 6600b. A portion of the barbed mesh 6402 and a portion of the base membrane 6415 are positioned on one of the transected tissue ends 6600a or 6600b, and another portion of the barbed mesh 6402 and another portion of the base membrane 6415 are positioned on the other one of the transected tissue ends 6600a or 6600b. The transected tissue ends 6600a and 6600b may be arranged so that the end regions of the transected tissue ends 6600a and 6600b are positioned on the barbed mesh 6402 and on the base membrane 6415. The transected tissue ends 6600a and 6600b may be positioned relative to one another so that a gap G₆₆ that is formed between the transected tissue ends 6600a and 6600b may be approximately 0 mm or may be less than about 5 mm. Then, the barbed mesh 6402 and the base membrane 6415 may be wrapped to extend around the outer surface of each transected tissue end 6600a and 6600b. In some aspects, the mesh 6402 may first be wrapped around the transected tissue ends, and then the base membrane 6415 may be wrapped around the tissue ends, overlaying the mesh 6402. In still other aspects, the wrapping may occur substantially simultaneously. In still other aspects, the mesh 6402 may be affixed to the base membrane 6415, e.g., in any of the ways described above in regards to affixing barbed sutures to the base membrane, and thus the tissue repair device 6400 may be wrapped around the tissue ends as a whole. Although not shown, in some aspects, a separate base membrane 6415 may first be wrapped around the tissue ends, and then a separate mesh 6402 may be wrapped over the base membrane 6415, holding the base membrane 6415 and the underlying tissue in place.

Wrapping the tissue repair device 6400 may include physically moving (e.g., rolling, twisting, or pulling) the barbed mesh 6402 and the base membrane 6415, individually or together, and laying them on the outer surface of the transected tissue ends 6600a and 6600b, rolling the transected tissue ends 6600a and 6600b on the barbed mesh 6402 and the base membrane 6415 with the barbed mesh 6402 being interposed between the transected tissue ends 6600a and 6600b and the base membrane 6415, or a combination thereof. In some aspects, ends of the barbed mesh 6402 and/or the base membrane 6415 may be secured after wrapping, for example, using a suture, an adhesive, a gel, or by capillary action. By this configuration, the tissue repair device 6400, including the barbed mesh 6402 and the base membrane 6415, may secure the transected tissue ends 6600a and 6600b together so that a tissue repair process may occur. More specifically, barbs 6410 of the barbed mesh 6402 may protrude into or otherwise engage with the outer surface of each of the transected tissue ends 6600a and 6600b, and the barbs 6410 of the barbed mesh 6402 and the base membrane 6415, may inhibit the transected tissue ends 6600a and 6600b from moving apart.

As discussed above, use of the base membrane 6415 with the barbed mesh 6402 may provide additional protection for the tissues being repaired, may act as a barrier to prevent the barbed mesh 6402 from rubbing on surrounding structures, or may be used to deliver one or more drugs or chemicals to the tissue repair site. If the tissue being repaired is a nerve tissue, then the presence of the base membrane 6415 may inhibit axonal escape as the axons regrow. The base membrane 6415 may further provide one or more of protection of the neurotrophy formed by the mesh 6402, preventing ingrowth of fibrotic tissue, and preventing outgrowth of axons, etc. Further, use of the base membrane 6415 may be configured for attachment of tissue ends with a gap therebetween to provide a covering over the gap region.

Although transected tissue ends are discussed in reference to FIGS. 64-66, the tissue repair devices of this embodiment may be applied to injured tissues of any type, whether fully transected or not, e.g., for protection or to allow the underlying tissue to heal, or simply to secure tissue for any other suitable reason, unrelated to healing. For example, instead of two tissue ends being brought together, embodiments of the disclosure may wrap around or otherwise cover a portion of tissue that is intact, to promote repair or to otherwise protect the underlying tissue.

E. Base Membrane with Barbed Mesh

FIG. 67 is a schematic view of a configuration of the tissue repair device 6400, including the barbed mesh 6402 and the base membrane 6415 during a tissue repair process, according to still another embodiment. In particular, FIG. 67 shows the tissue repair device 6400, including the barbed mesh 6402 and the base membrane 6415, and two transected tissue ends 6700a and 6700b. Similar to the configuration shown in FIG. 66, in this configuration, two transected tissue ends 6700a and 6700b may be placed on the barbed mesh 6402 and the base membrane 6415, but in this configuration, a gap G₆₇ therebetween that may be greater than approximately 0 mm. The gap G₆₇ between transected tissue ends 6700a and 6700b may occur, for example, due to a tendency of tissue, such as nerve tissue, to retract, as tissue tends to be under an amount of tension in vivo. As a result, once the tissue is transected and the tension is relieved, the transected tissue ends 6700a and 6700b may pull away from each other. In addition, an injury to tissue which causes transection of the tissue may similarly create a gap G₆₇ between tissue ends 6700a and 6700b. Still further, a surgeon may trim material off of transected tissue ends 6700a and 6700b to remove damaged or injured tissue and expose healthy tissue. Forcefully bringing transected tissue ends 6700a and 6700b together, to close a gap G₆₇, could exert tension on tissues, which, in turn, may detrimentally impact tissue regeneration.

The gap G₆₇ may be greater than approximately 0 mm, for example, up to about 10 mm or up to about 5 mm. For example, the gap may be about 0 mm to about 10 mm, about 0 mm to about 8 mm, about 1 mm to about 10 mm, about 1 mm to about 8 mm, about 2 mm to about 8 mm, about 3 mm to about 7 mm, about 3 mm, about 4 mm, about 5 mm, about 6 mm, about 7 mm, etc. The wrapping and securing of the barbed mesh 6402 and the base membrane 6415 of this configuration may be the same as that shown and described with respect to FIG. 66.

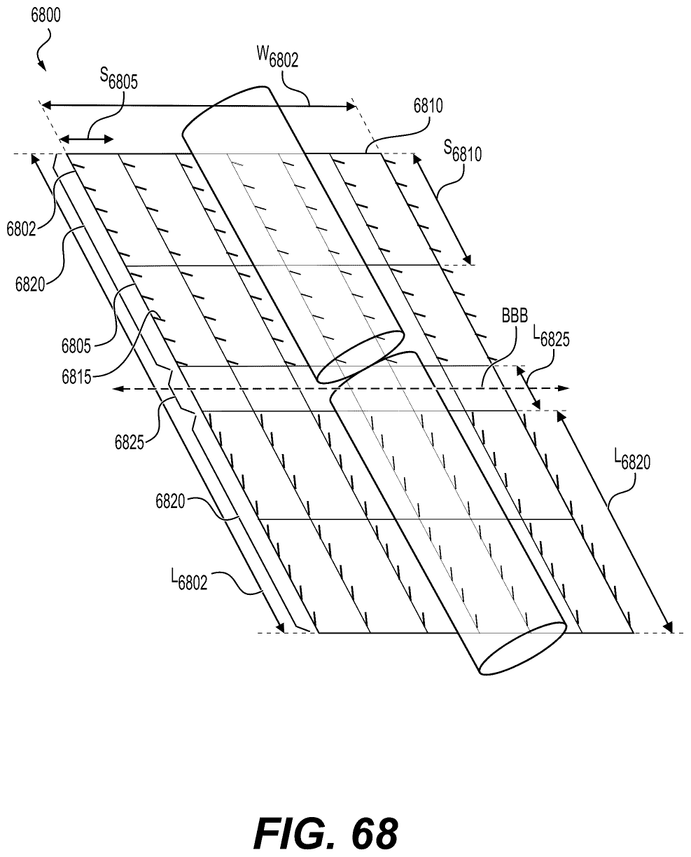

FIG. 68 is a schematic view of another barbed mesh 6802, as part of a tissue repair device 6800, with no base membrane. As with the embodiment shown in FIG. 64A, the barbed mesh 6802 is formed of a plurality of barbed sutures 6805, but in this embodiment, the barbed mesh 6802 also includes non-barbed sutures 6810. The barbed sutures 6805 are arranged longitudinally in FIG. 68, and the non-barbed sutures 6810 are arranged laterally. Each of the barbed sutures 6805 may include one or more barbs 6815 extending from a surface of the barbed suture 6805 configured to contact a tissue. In the embodiment shown in FIG. 68, each barbed suture 6805 has a plurality of barbs 6815 along a length thereof, with barbed portions 6820 including the barbs 6815, and a non-barbed portion 6825 having no barbs. One of the barbed portions 6820 on one side of a central axis BBB of the barbed mesh 6802, may have barbs 6815 pointing in a first direction, e.g., pointing towards the central axis BBB, and the other of the barbed portions 6820, on another side of the central axis BBB, may have barbs 6815 pointing in a second direction, e.g., towards the central axis BBB. That is, the barbed mesh 6802 of this embodiment may have barbs 6815 pointing in one of two directions. In other embodiments, barbed meshes may have barbs pointing in more than two direction. The barbs 6815 may be evenly spaced, within the barbed portions 6820 of the barbed mesh 6802.

Dimensions of the barbed mesh 6802, including dimensions of the barbed portions 6820 and the non-barbed portion 6825, and spacing of the barbed sutures 6805 and spacing of the non-barbed sutures 6810 from adjacent sutures, may depend upon one or both of the type of barb (e.g., the directional arrangement of the barbs, the material of the barbs, the shape of the barbs, the size of the barbs, etc.) used, the type of tissue and/or procedure with which the tissue repair device is intended to be applied, and the targeted anatomical region or tissue to be wrapped and repaired using the tissue repair device 6800. For example, a length L₆₈₀₂ of the barbed mesh 6802 may be about 35 mm, a width W₆₈₀₂ of the barbed mesh 6802 may be about 15 mm, a length L₆₈₂₀ of the barbed portions 6820 may be about 15 mm, a length L₆₈₂₅ of the non-barbed portion 6825 may be about 5 mm, a spacing S₆₈₀₅ between the barbed sutures 6805 may be about 2.5 mm or about 3 mm, and a spacing S₆₈₁₀ between non-barbed sutures 6810 may be about 7.5 mm. These dimensions, however, serve as an example only and are not limiting. Depending on the use case and factors described above, other dimensions may be suitable.

The barbed sutures 6805 may be formed of polydioxanone (PDO), and may be one of suture types 3-0, 4-0, or 5-0, for example, although any of the materials or sizes described above as suitable for a barbed suture may be used. The non-barbed sutures 6810 may be formed of polydioxanone (PDO), and may be suture type 5-0, for example, although any of the materials or sizes described above as suitable for a barbed suture may be used. In a case in which tissue to be secured using the barbed mesh 6802 is nerve tissue, a size of the barbed sutures 6805 may be based on a size of the nerve tissue. For example, if the nerve tissue has a diameter of about 5 mm and a circumference of about 15.7 mm, the barbed suture 6805 may be a 3-0 sized thread, with a diameter between about 300 μm and 339 μm. As another example, if the nerve tissue has a diameter of about 4 mm, and a circumference of about 12.6 mm, the barbed suture 6805 may be 5-0 sized thread, with a diameter of between about 150 μm and 199 μm.

Each barbed suture 6805 and non-barbed suture 6810 may be woven together or overlaid with the other sutures, or the sutures may be connected to one another at intersection points. Alternatively, the sutures may be formed as a uniform mesh, instead of being woven. In some aspects, the barbed mesh 6802 may be 3D printed, woven from individual sutures, or formed in any other suitable manner.

Similar to use of the tissue repair device 6400, the tissue repair device 6800 may be wrapped around a tissue or placed on a tissue, e.g., a transected nerve. The barbs 6815 may hold the barbed mesh 6802 on the tissue and/or may hold tissues in place relative to one another. The holding or fixation of tissue, including nerve tissue or tissue types other than nerve tissue, may be achieved by the multi-directional arrangement of barbs 6815, as described above. The tissue repair device 6800 may be used by itself, or may be overlaid with a base membrane, as with the embodiment of FIGS. 66 and 67. In still other aspects, the barbed mesh 6802 may be attached to a base membrane, as described above.

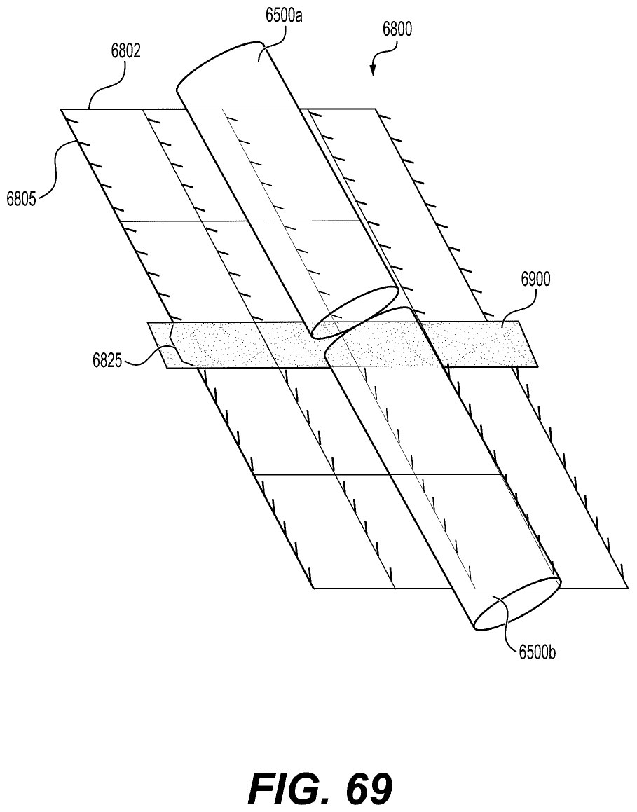

FIG. 69 shows the tissue repair device 6800 with a strip of base membrane 6900 laid atop the non-barbed portion 6825 of the barbed sutures 6805 of the barbed mesh 6802, according one embodiment. In this embodiment, a gap formed between tissue ends 6500a and 6500b and the extreme ends of the tissue ends 6500a and 6500b may be positioned on top of the strip of baes membrane 6900. The strip of base membrane 6900 may have one or more of the properties of the base membrane 3900 described above. Although the strip of base membrane 6900 is shown as being layered between the barbed mesh 6802 and tissue ends 6500a and 6500b, the strips of base membrane 6900 may alternatively be laid on an opposite side of the non-barbed portion 6825 of the barbed mesh 6802, sandwiching the non-barbed portion 6825 between the strip of base membrane 6900 and tissue ends 6500a and 6500b.

Although transected tissue ends are discussed in reference to FIGS. 67, 68, and 69, the tissue repair devices of these embodiments may be applied to injured tissues of any type, whether fully transected or not, e.g., for protection or to allow the underlying tissue to heal, or simply to secure tissue for any other suitable reason, unrelated to healing. For example, instead of two tissue ends being brought together, embodiments of the disclosure may wrap around or otherwise cover a portion of tissue that is intact, to promote repair or to otherwise protect the underlying tissue.

F. Base Membrane with Barbs

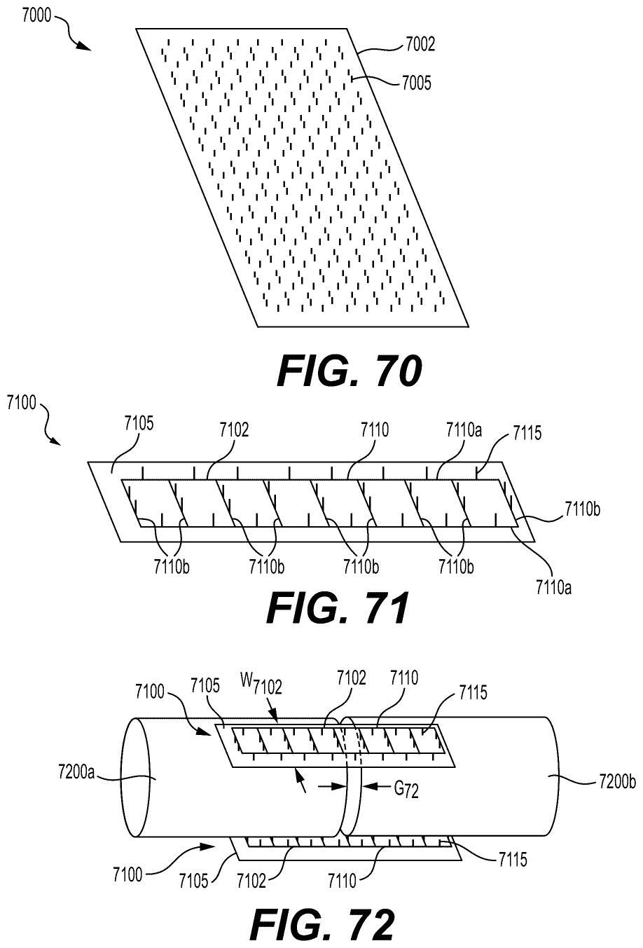

FIG. 70 is a schematic view of a tissue repair device 7000 with a base membrane 7002 and a plurality of barbs 7005 extending from a surface of the base membrane 7002 that is configured to contact with a tissue. Properties of the base membrane 7002 may be the same as those noted above with respect to base membrane 6415. The barbs 7005 may be formed of the same material or a different material from which the base membrane 7002 is formed. In some aspects, the barbs 7005 may be formed by laser cutting or embossing the base membrane 7002, according to one embodiment, or may be ceramic barbs 7005 attached to the base membrane 7002, according to another embodiment, or a combination thereof. The tissue repair device 7000, including the base membrane 7002 with barbs 7005, may be used in a similar or the same manner as the tissue repair device 6400 of the embodiment shown in FIGS. 64 to 67. The barbs 7005 may be arranged on the base membrane 7002 in a grid configuration or other pattern. Other configurations, including randomly spaced apart barbs 7005, may alternatively be used. Further, although FIG. 70 depicts the barbs 7005 projecting from the entire base membrane 7002, barbs 7005 may only project from a portion of base membrane 7002, e.g., along one or more edges of base membrane 7002, along a half or less than a half of base membrane 7002, etc., as described above in relation to the portion of a base membrane that may include barbed structures. In an embodiment in which the barbs 7005 are ceramic barbs, the barbs 7005 may be formed of resorbable ceramic, such as bioglass, for example. If the barbs 7005 and the base membrane 7002 are formed of different materials, e.g., different resorbable materials, the barbs 7005 and the base membrane 7002 may have different mechanical properties. For example, the base membrane 7002 may be more flexible, whereas the barbs 7005 may be relatively more rigid in order to extend into and engage with the tissue to which the tissue repair device 7000 is applied. In some aspects, the base membrane 7002 may be configured to resorb at a different speed (e.g., faster or slower) compared to the barbs 7005, or they may be configured to resorb at substantially the same time.

### G. Barbed Strip and Base Membrane

FIG. 71 is a schematic view of a tissue repair device 7100 including a barbed strip 7102 formed of barbed sutures 7110, which may be similar to or the same as those described above, attached to or embedded in a base membrane 7105, according to one embodiment. Properties of the base membrane 7105 may be the same as those noted above with respect to base membrane 3900. The barbed strip 7102 may be formed of a plurality of laterally and longitudinally arranged barbed sutures 7110 with barbs 7115 extending from a surface of each barbed suture 7110 configured to contact with a tissue. In other aspects, the lateral sutures may not include barbs, and only the longitudinally arranged barbed sutures may include barbs. In some aspects, the plurality of barbed sutures 7110 may be arranged in a grid pattern. For example, the barbed strip 7102 may include two or more longitudinal barbed sutures 7110a and two or more lateral barbed sutures 7110b, the two longitudinal barbed sutures 7110a having a relatively greater length than the two or more lateral barbed sutures 7110b, thus forming the barbed strip 7102. Each barbed suture 7110 may include one or more barbs 7115. Although two longitudinal barbed sutures 7110a are described in reference to FIG. 71, additional longitudinal barbed sutures may be included, and greater or fewer lateral barbed sutures 7110b may be included, as well. Each barbed suture 7110 may be a separate barbed suture, e.g., woven together or overlaid with the other barbed sutures 7110, or the barbed sutures 7110 may be connected to one another where two or more barbed sutures 7110 intersect, or may be one, uniform mesh instead of woven or overlaid barbed sutures. In some aspects, the barbed strip 7102 may be 3D printed, woven from barbed sutures 7110, or formed in any other suitable manner.

The barbed sutures 7110 may be attached to the base membrane 7105 in any suitable manner. For example, barbed sutures 7110 may be attached using a glue or an adhesive, as described above. As another example, the barbed sutures 7110 may be attached physically, e.g., by weaving or sewing the barbed sutures 7110 through the base membrane 7105, or the barbed sutures 7110 may be hooked onto the base membrane 7105 by making J-shaped or S-shaped cuts in the base membrane 7105 or making small holes in the side of the base membrane 7105, threading the barbed sutures 7110 through the cuts or holes, and tying knots in the ends of the barbed sutures 7110 once threaded through the cuts or holes, as also described above. As another example, base membrane 7105 may be attached to the barbed sutures 7110 by inserting the base membrane 7105 into a thin slit or cut through the barbed sutures 7110 or attaching two barbed sutures 7110 with glue on their ends and inserting the base membrane 7105 through the attached barbed sutures 7110, as is also described above. As still another example, the barbed sutures 7110 may be attached using heat, such as by melting the barbed sutures 7110 at specific locations as the barbed sutures 7110 lay atop the base membrane 7105, as described above.

As still a further example, the barbed sutures 7110 may be attached to the base membrane 7105 using etching, in which barbed sutures 7110 are arranged in a desired orientation, and a material that forms the base membrane 7105 (e.g., a resorbable polymer, or any of the other materials listed above as being used to form base membrane 3900) is cast around the barbed sutures 7110, such that the barbed sutures 7110 are completely covered in the material. Then the material may be selectively etched to expose the barbed sutures 7110, on at least one end, and another end of the material may be sliced to cut a thin film, thereby forming the tissue repair device 7100, with the barbed sutures 7110 embedded within the base membrane 7105. The tissue repair device 7100 may include one or more barbed strips 7102, which may be applied to tissue to attach tissues to one another.

a. Use of Barbed Strip and Base Membrane

FIG. 72 is a schematic view showing use of two tissue repair devices 7100, as shown in FIG. 71, during a tissue repair process, according to one embodiment. In particular, FIG. 72 shows two tissue repair devices 7100 and two transected tissue ends 7200a and 7200b. The two barbed strips 7102 of the tissue repair devices 7100 may be placed on the transected tissue ends 7200a and 7200b so that a portion of each barbed strip 7102 is positioned on one of the transected tissue ends 7200a and 7200b, and another portion of each barbed strip 7102 is positioned on the other one of the transected tissue ends 7200a and 7200b, so as to ‘tape’ the transected tissue ends 7200a and 7200b together. The transected tissue ends 7200a and 7200b may be arranged so that the end regions of the transected tissue ends 7200a and 7200b are positioned on the barbed strips 7102. The transected tissue ends 7200a and 7200b may be positioned relative to one another so that a gap G₇₂ formed between the transected tissue ends 7200a and 7200b may be approximately 0 mm or may be less than about 5 mm. Although FIG. 72 depicts two barbed strips 7102 placed on opposing sides of the transected tissue ends 7200a and 7200b to ‘tape’ the transected tissue ends 7200a and 7200b together, one or more than two barbed strips 7102 may be used.

When the barbed strips 7102 are placed on the transected tissue ends 7200a and 7200b (or vice versa), the width W₇₁₀₂ of the barbed strip 7102 may lay on or adhere to a portion of the outer surfaces of the transected tissue ends 7200a and 7200b, as shown in FIG. 72. That is, the barbed strips 7102 may extend around only a portion of the surface of the transected tissue ends 7200a and 7200b, as the width W_{STRIP} of each barbed strip 7102 may be less than a width of the tissue ends 7200a and 7200b. By this configuration, the barbed strips 7102 may secure the transected tissue ends 7200a and 7200b relative to one another so that a tissue repair process may occur. More specifically, barbs 7115 of the barbed strips 7102 may protrude into or otherwise engage with the outer surface of each of the transected tissue ends 7200a and 7200b, and the barbs 7115 of the barbed strips 7102 may inhibit the transected tissue ends 7200a and 7200b from moving apart.

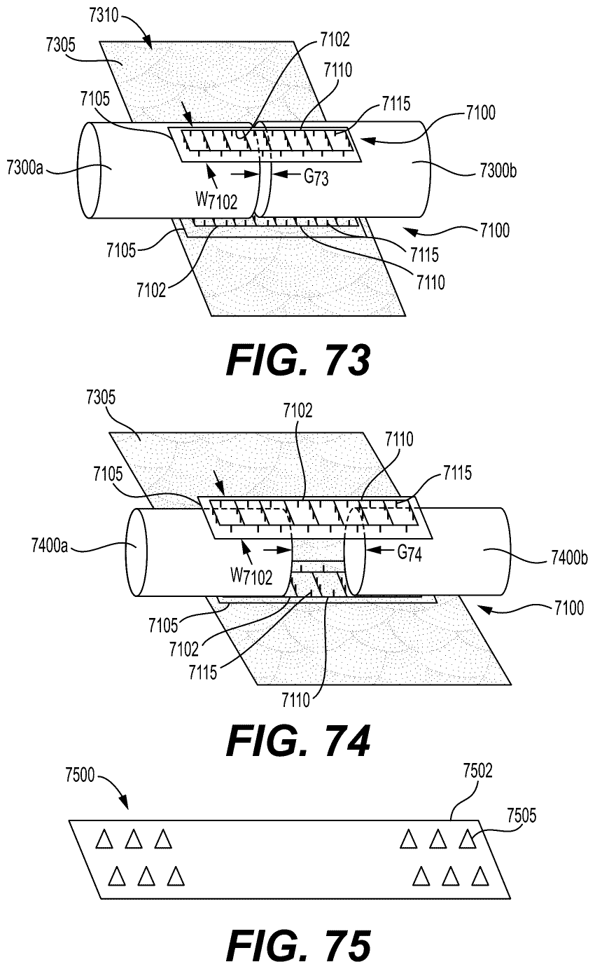

FIG. 73 is a schematic view of a use of two tissue repair devices 7100, as shown in FIG. 71, and a separate base membrane 7305, relatively larger than the base membrane 7105 that forms a part of tissue repair device 7100, during a tissue repair process, according to another embodiment. In particular, FIG. 73 shows two barbed strips 7102, the separate base membrane 7305, and two transected tissue ends 7300a and 7300b. Properties of the separate base membrane 7305 may be the same as those of the base membrane 3900, described above.

Similar to the configuration shown in FIG. 72, in this configuration, barbed strips 7102 may be placed on two transected tissue ends 7300a and 7300b (or vice versa), but in this embodiment, once the barbed strips 7102 are in place, the separate base membrane 7305 may be wrapped around the barbed strips 7102 and the tissue ends 7300a and 7300b. A portion of each barbed strip 7102 may be positioned on one of the transected tissue ends 7300a and 7300b, and another portion of each barbed strip 7102 may be positioned on the other one of the transected tissue ends 7300a and 7300b. The transected tissue ends 7300a and 7300b may be arranged so that the end regions of the transected tissue ends 7300a and 7300b are positioned on the barbed strips 7102, and the portions of the transected tissue ends 7300a and 7300b and the barbed strips 7102 are positioned on the separate base membrane 7305. The transected tissue ends 7300a and 7300b may be positioned relative to one another so that a gap G₇₃ formed between the transected tissue ends 3200a and 3200b may be approximately 0 mm or may be less than about 5 mm. Although this embodiment depicts two barbed strips 7102 being used in the procedure, one or more than two barbed strips 7102 may be used.

A width W₇₁₀₂ of the barbed strips 7102 may be such that, when the barbed strips 7102 are placed on the transected tissue ends 7300a and 7300b, the entire width W₇₁₀₂ of each barbed strip 7102 lays on or adheres to a portion of the outer surfaces of the transected tissue ends 7300a and 7300b, as shown in FIG. 73. That is, the barbed strips 7102 may extend around only a portion of the external surface of the transected tissue ends 7300a and 7300b, as the width W₇₁₀₂ may be less than a width of the tissue ends 7300a and 7300b. Then, the separate base membrane 7305 may be wrapped around the entire surface of each transected tissue end 7300a and 7300b and around the barbed strips 7102.

Wrapping the transected tissue ends 7300a and 7300b in the separate base membrane 7305 may include physically moving (e.g., rolling, twisting, or pulling) the separate base membrane 7305 and laying it on the surface of the transected tissue ends 7300a and 7300b, rolling the transected tissue ends 7300a and 7300b on the separate base membrane 7305, or a combination thereof. In some aspects, ends of the separate base membrane 7305 may be secured after wrapping, for example, using a suture, an adhesive, a gel, or capillary action, for example. By this configuration, the tissue repair device 7100, including the barbed strips 7102 and the separate base membrane 7305, may secure the transected tissue ends 7300a and 7300b together so that a tissue repair process may occur. More specifically, barbs 7115 on the barbed strips 7102 may protrude into or otherwise engage with the outer surface of each of the transected tissue ends 7300a and 7300b, and the barbs 7115 of the barbed strip 7102, and the separate base membrane 7305, may inhibit the transected tissue ends 7300a and 7300b from moving apart. If the transected tissue ends 7300a and 7300b are nerve ends, then by covering the transected tissue ends 7300a and 7300b held together by the barbed strips 7102 with the separate base membrane 7305, it may be possible to prevent axons that may grow from one or both of the transected tissue ends 7300a and 7300b from escaping, that is, from extending outward and away from the other one of the transected tissue ends 7300a and 7300b. Although two the tissue repair devices 7100 are depicted in use in FIG. 73, one or more than two tissue repair devices 7100 may be used.

FIG. 74 depicts use of two tissue repair devices 7100 and the separate base membrane 7305 during a tissue repair process, according to still another embodiment. In particular, FIG. 74 shows the barbed strips 7102 applied to two transected tissue ends 7400a and 7400b. As described in reference to FIG. 73, the separate base membrane 7305 may also be used in this procedure, in which a larger gap is left between the two transected tissue ends 7400a and 7400b. Similar to the configuration shown in FIG. 73, in this configuration, barbed strips 7102 may be applied to two transected tissue ends 7400a and 7400b (or vice versa), but in this configuration, the transected tissue ends 7400a and 7400b may be arranged relative to one another with a gap G₇₄ therebetween, for the reasons discussed above with respect to gap G₆₇. The gap G₇₄ may be greater than approximately 0 mm, for example, up to about 10 mm or up to about 5 mm. For example, the gap G₇₄ may be about 0 mm to about 10 mm, about 0 mm to about 8 mm, about 1 mm to about 10 mm, about 1 mm to about 8 mm, about 2 mm to about 8 mm, about 3 mm to about 7 mm, about 3 mm, about 4 mm, about 5 mm, about 6 mm, about 7 mm, etc.

The wrapping and securing of the separate base membrane 7305 of this configuration may be the same as that shown and described with respect to FIG. 73. If the transected tissue ends 7400a and 7400b are nerve ends, then use of the separate base membrane 7305 over the transected tissue ends 7400a and 7400b and the barbed strips 7102 may prevent axons that may grow from one or both of the transected tissue ends 7400a and 7400b from escaping, that is, from extending outward and away from the other one of the transected tissue ends 7400a and 7400b and outward from the gap G₇₄. Although FIG. 74 shows two tissue repair devices 7100 being used on the transected tissue ends 7400a and 7400b, one or more than two barbed strips 7102 may be used.

Although transected tissue ends are discussed in reference to FIGS. 71-74, the tissue repair devices of these embodiments may be applied to injured tissues of any type, whether fully transected or not, e.g., for protection or to allow the underlying tissue to heal, or simply to secure tissue for any other suitable reason, unrelated to healing. For example, instead of two tissue ends being brought together, embodiments of the disclosure may wrap around or otherwise cover a portion of tissue that is intact, to promote repair or to otherwise protect the underlying tissue.

H. Strip of Base Membrane with Barbs

FIG. 75 is a schematic view of a tissue repair device 7500 including a strip of base membrane 7502 with barbs 7505 extending from a surface of the base membrane 7502 configured to contact tissue. One or more properties of the base membrane 7502 may be the same as those noted above with respect to base membrane 3900. In some aspects, the barbs 7505 may extend from end regions of the base membrane 7502. In some aspects, a central region of the base membrane 7502 may not include barbs 7505, while in other aspects, the central region of the base membrane 7502 may also include barbs 7505. The barbs 7505 may be configured to engage the tissue to which they are applied. The tissue repair device 7500, including the base membrane 7502 with barbs 7505, may be used in a similar manner as the tissue repair device 7100, including the barbed strips 7102, of the embodiment shown in FIGS. 71 to 74. When used according to the methods of application shown in FIGS. 72 to 74, the barbs 7505 at the end regions of the base membrane 7502 may contact and engage two different regions or ends of tissues. In configurations of the base membrane 7502 for which no barbs 7505 are located in a central region of the base membrane 7502, this barb-less central region may be positioned to generally align with a gap between the tissue (e.g., gaps G₇₂, G₇₃, or G₇₄). As described above with reference to these figures, one or more tissue repair devices 7500 may be used in a similar manner as barbed strips 7102, including with or without a separate base membrane overlaying the tissue repair devices 7500.

The barbs 7505 may be arranged in rows, columns, or in a grid configuration, evenly spaced apart. Other configurations, including randomly spaced apart barbs 7505 or offset bards 7505, may be used. The barbs 7505 may have a shape similar to that of a barb of one of the embodiments described herein, or may be different. The tissue repair device 7500 may be formed of a resorbable polymer or metal, for example, and may be a micro-injection molded strip with molded barbs, or may be 3D printed, or formed in any other suitable manner. In some aspects, the barbs 7505 may be laser cut or embossed in the base membrane 7502, which, in this case, may be formed of the same material. In other aspects, the barbs 7505 may be formed separate from the base membrane 7502 and then attached to the base membrane 7502, in any of the ways described above. In some aspects, the barbs 7505 may be formed of a different resorbable material than the base membrane 7402. If the barbs 7505 and the base membrane 7502 are formed of different materials, e.g., different resorbable polymers or metals, the barbs 7505 and the base membrane 7502 may have different mechanical properties. For example, the base membrane 7502 may be more flexible, whereas the barbs 7505 may be relatively more rigid in order to extend into and engage with the tissue. In some aspects, the strip of base membrane 7502 may be configured to resorb at a different speed (e.g., faster or slower) compared to the barbs 7505, or at substantially the same speed.

### I. Base Membrane and Ceramic Barbs

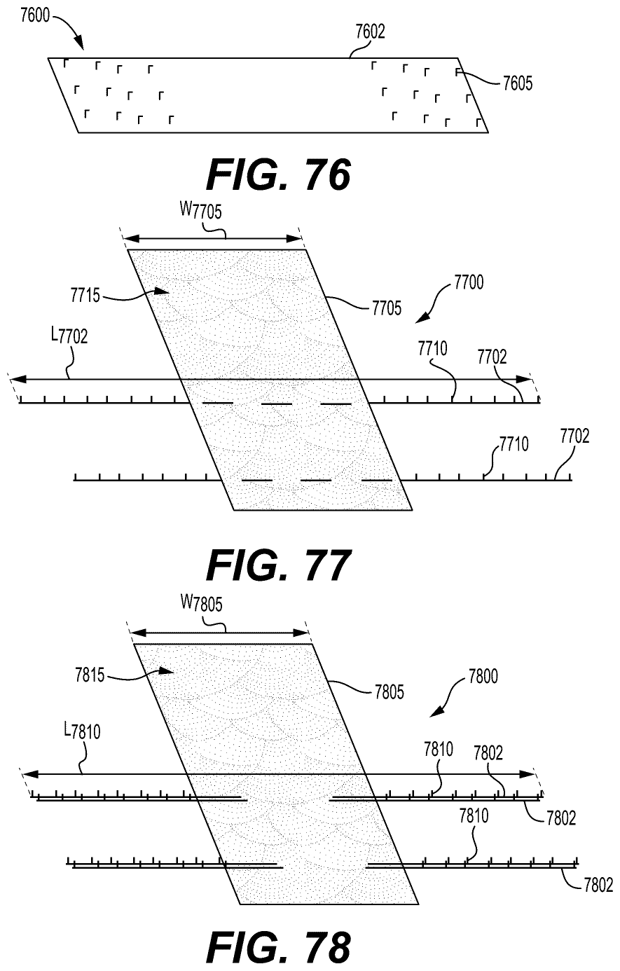

FIG. 76 is a schematic view of a tissue repair device 7600, including a base membrane 7602 with ceramic barbs 7605 attached to or embedded within the base membrane 7602, according to another embodiment. One or more properties of the base membrane 7602 may be the same as those noted above with respect to base membrane 3900. The ceramic barbs 7605 may extend from a surface of the base membrane 7602 configured to contact tissue and may be configured to engage the tissue to which they are applied. In some aspects, the barbs 7605 may extend from end regions of the base membrane 7602. In some aspects, a central region of the base membrane 7602 may not include barbs 7605, while in other aspects, the central region of the base membrane 7602 may also include barbs 7605. The base membrane 7602 with ceramic barbs 7605 may be used in a similar or the same manner as the barbed strip 7102 of the tissue repair device 7100 of the embodiment shown in FIGS. 71 to 74 and/or the base membrane 7502 of the tissue repair device 7500 of the embodiment shown in FIG. 75. When used according to the methods of application shown in FIGS. 72 to 74, the barbs 7505 at the end regions of the strip of base membrane 7602 may contact and engage end regions of two different tissues or two different regions of tissue. In configurations of the base membrane 7602 for which no barbs 7605 are located in a central region of the strip of base membrane 7602, this barb-less central region may be positioned to generally align with a gap between the tissue ends (e.g., gaps G₇₂, G₇₃, or G₇₄). As described above with reference to these figures, one or more base membranes 7602 may be used in a similar manner as barbed strips 7102 and/or the base membrane 7502.

Similar to the embodiment shown in FIG. 75, the ceramic barbs 7605 of this embodiment may be arranged in rows, columns, or in a grid configuration, evenly spaced apart. Other configurations, including randomly spaced apart barbs 7605 or offset barbs 7605, may be used. The barbs 7605 may have a shape similar to that of a barb of one of the embodiments described herein, or may be different. In an embodiment in which the barbs 7605 are ceramic barbs, the barbs 7605 may be formed with resorbable ceramic, such as bioglass, for example. The base membrane 7602 may be formed of a resorbable polymer, for example. If barbs 7605 and the base membrane 7602 are formed of different materials, e.g., different resorbable materials, the barbs 7605 and the base membrane 7602 may have different mechanical properties. For example, the strip of base membrane 7602 may be more flexible, whereas the barbs 7605 may be relatively more rigid in order to extend into and engage with the tissue. In some aspects, the base membrane 7602 may be configured to resorb at a different speed (e.g., faster or slower) compared to the barbs 7605, or at a substantially similar speed. In one aspect, to form the tissue repair device 7600, barbs 7605 may be placed, e.g., in a container, in a desired configuration or orientation, and a material that forms the base membrane 7602, such as a resorbable polymer, may be cast around the barbs 7605 placed in the container, such that the material completely covers the barbs 7605. Then, the material may be selectively etched to expose the barbs 7605 on one end, and another end of the material may be sliced to cut a thin film, thereby forming the tissue repair device 7600, with the barbs 7605 embedded within the base membrane 7602. The selective etching of the material may be performed to produce barbs 7605 of a predetermined height.

### J. Barbed Sutures and Base Membrane

FIG. 77 is a schematic view of a tissue repair device 7700 including two barbed sutures 7702 and a base membrane 7705, according to one embodiment. Properties of the base membrane 7705 may be the same as those noted above with respect to base membrane 3900. In particular, FIG. 77 shows two barbed sutures 7702 with barbs 7710 extending from a surface of each barbed suture 7702 configured to contact with a tissue, and the barbed sutures 7702 extending longitudinally. FIG. 77 also shows the base membrane 7705. The barbed sutures 7702 of this embodiment may be referred to as anchors, and may be arranged individually, that is, the barbed sutures 7702 may be arranged spaced apart from one or more additional barbed sutures 7702 on the base membrane 7705. The barbed sutures 7702 may have a longitudinal length L₇₇₀₂ that is greater than a width W₇₇₀₅ of the base membrane 7705 such that the barbed sutures 7702 extend out from edges of the base membrane 7705. Although FIG. 77 shows two barbed sutures 7702, the tissue repair device 7700 may include one or more than two barbed sutures 7702. Additionally, the barbed sutures 7702 may be located at one or more end regions of the base membrane 7705, at a central region and at one or more end regions of the base membrane 7705 (as show in FIG. 77), only at a central region of the base membrane 7705, or may be spaced along the base membrane 7705 in both central and end regions. Although FIG. 77 depicts the barbed sutures 7702 extending generally perpendicular to the base membrane 7705, with the longest edge of the base membrane 7705 perpendicular to the length of the barbed sutures 7702, other relative orientations of the base membrane 7705 and the barbed sutures 7702 are possible. For example, the barbed sutures 7702 may extend generally parallel with the longest edge of the base membrane 7705, or the barbed sutures 7702 may extend at an angle of greater than or less than 90 degrees relative to an edge of the base membrane 7705.

The barbed sutures 7702 may be attached to the base membrane 7705 in any suitable manner, as described above. For example, the barbed sutures 7702 may be woven or sewn through or to the base membrane 7705, as shown in FIG. 77, or they may pass through defined holes in the base membrane 7705, as shown in FIG. 78, or they may be sandwiched between two or more layers of multi-layered base membranes 7705. The barbed sutures 7702 may include barbs 7710 on some portions, and no barbs 7710 in portions that extend across the base membrane 7705. The portions of the barbed sutures 7702 with barbs 7710 may extend outward from the base membrane 7705, as shown. By this arrangement, in some aspects, none of the barbs 7710 may penetrate through the base membrane 7705.

During use, the base membrane 7705 with barbed sutures 7702 of the tissue repair device 7700 may be applied in a similar manner as the tissue repair device 7100 described in regards to FIGS. 72 to 74, except that instead of having separate barbed strips that are applied to the tissue ends and then wrapping the strips and tissue ends with a separate base membrane, the barbed sutures 7702 may essentially act as the barbed strips 7102, and the base membrane 7705 may act as an integrated base membrane that is wrapped around the tissue. Instead of applying separate components as discussed in FIGS. 73 and 74, the barbed sutures 7702 may be integrated with the base membrane 7705 in the form of barbed sutures 7702. Accordingly, end regions of tissues may be secured relative to one another with the barbed ends of barbed sutures 7702, and the base membrane 7705 may be wrapped around the secured tissue ends. To secure the barbed sutures 7702 to the tissue ends or portions, the barbed sutures 7702 may be engaged with an outer surface of the tissue end regions before and/or as the base membrane 7705 is being wrapped around the tissue ends. In some aspects, the barbed sutures 7702 may be spaced along base membrane 7705 such that barbed sutures 7702 may be positioned on generally opposite sides or ends of tissue when applied, or they may be positioned along different portions of the tissue, as the base membrane 7705 may be wrapped around the tissue.

Although transected tissue ends are discussed in reference to FIG. 77, the tissue repair devices of this embodiment may be applied to injured tissues of any type, whether fully transected or not, e.g., for protection or to allow the underlying tissue to heal, or simply to secure tissue for any other suitable reason, unrelated to healing. For example, instead of two tissue ends being brought together, embodiments of the disclosure may wrap around or otherwise cover a portion of tissue that is intact, to promote repair or to otherwise protect the underlying tissue.

### K. Sets of Side-by-Side Sutures and Base Membrane

FIG. 78 is a schematic view of a tissue repair device 7800 including two sets of side-by-side barbed sutures 7802, and a base membrane 7805, according to another embodiment. In particular, FIG. 78 shows two sets of barbed sutures 7802 with barbs 7810, the barbed sutures 7802 extending longitudinally, and the base membrane 7805. One or more properties of the base membrane 7805 may be the same as those noted above with respect to base membrane 3900. The barbed sutures 7802 of this embodiment may be arranged together in sets, with each set of barbed sutures 7802 including two or more individual barbed sutures 7802. Although sets of two barbed sutures 7802 are shown, larger groupings of barbed sutures 7802 are possible, such as sets of three barbed sutures 7802, sets of four barbed sutures 7802, etc.

Each set of barbed sutures 7802 may be connected to one another. For example, the sets of barbed sutures 7802 may be welded together or attached via coupling sutures, ties, bands, or any other suitable fasteners. The sets of barbed sutures 7802 may be arranged spaced apart from one or more additional sets of barbed sutures 7802 on the base membrane 7805. The sets of barbed sutures 7802 may be spaced along the base membrane 7805 in any of the arrangements or relative orientations as described above with regards to FIG. 77, base membrane 7705, and barbed sutures 7702.

The sets of barbed sutures 7802 may have a longitudinal length L₇₈₀₂ that is greater than a width W₇₈₀₅ of the base membrane 7805. Although FIG. 78 shows two sets of barbed sutures 7802, a single set of barbed sutures 7802 or more than two sets of barbed sutures 7802 may be used. The sets of barbed sutures 7802 may be attached to the base membrane 7805 in any of the ways described above. For example, the sets of barbed sutures 7802 may pass through defined holes in the base membrane 7805, as shown in FIG. 78, or they may be woven or sewn through the base membrane 7805, as shown in FIG. 77, or sandwiched between two layers of multi-layered base membranes 7805. And, similar to the embodiment shown in FIG. 77, the barbed sutures 7802 may include portions with barbs 7810 and portions with no barbs 7810 in locations extending across the base membrane 7805. The portions of the barbed sutures 7802 with barbs 7810 may extend outward from the base membrane 7805, as shown. By this arrangement, in some aspects, none of the barbs 7810 may penetrate through the base membrane 7805.

### L. Barbed Strips and Base Membrane

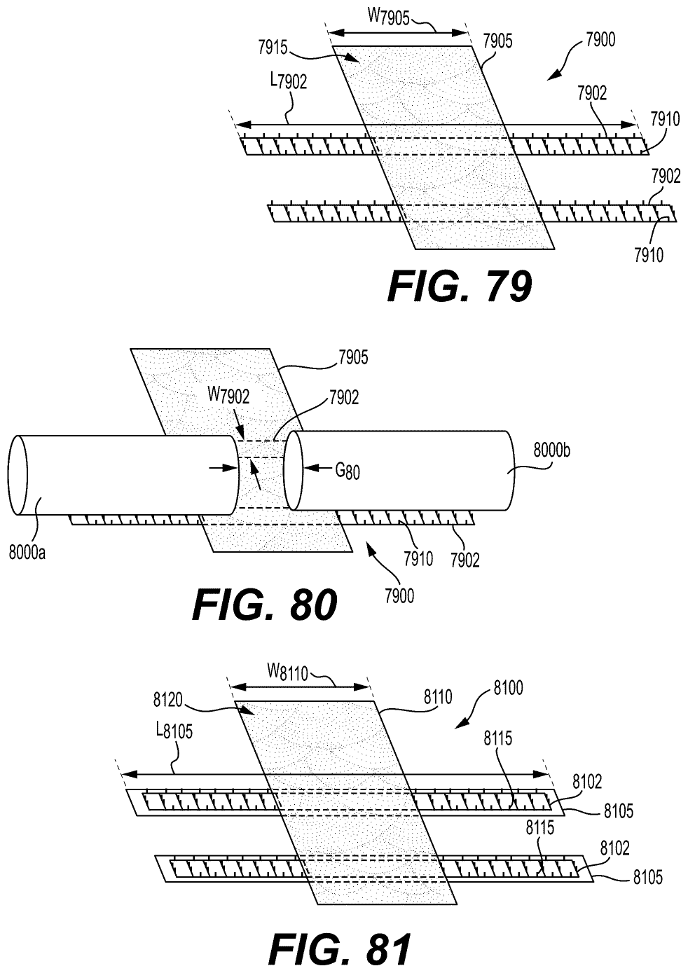

FIG. 79 is a schematic view of a tissue repair device 7900 having two barbed strips 7902 integrated with a base membrane 7905, according to yet another embodiment. In particular, FIG. 79 shows two barbed strips 7902 with barbs 7910 integrated with the base membrane 7905. One or more properties of the base membrane 7905 may be the same as those noted above with respect to base membrane 3900. In this embodiment, rather than integrating barbed sutures or sets of barbed sutures (FIGS. 77 and 78), strips or meshes, as described above with reference to FIGS. 71 to 74, may be integrated as part of the base membrane 7905. The barbed strips 7902 may be formed of a plurality of laterally and longitudinally arranged barbed sutures, or a combination of barbed and un-barbed sutures, or in other words, a plurality of barbed sutures, or a combination of barbed and un-barbed sutures, in a thin grid pattern, or a mesh. Each barbed strip 7902 may include one or more barbs 7910 extending from a surface of the barbed strip 7902 configured to contact with a tissue. The barbed strips 7902 may be arranged spaced apart from one or more additional barbed strips 7902 on the integrated base membrane 7905 in any suitable arrangement or orientation, as described above in reference to FIGS. 77 and 78. The barbed strips 7902 may have a longitudinal length L₇₉₀₂ that is greater than a width W₇₉₀₅ of the base membrane 7905. Although FIG. 79 shows the tissue repair device 7900 having two barbed strips 7902, a single barbed strip 7902 or more than two barbed strips 7902 may be included. The barbed strips 7902 may be sandwiched between two or more layers of multi-layer base membranes 7905, or may be sewn, woven, or passed through holes in the base membrane 7905, as shown in FIG. 79. And, similar to the embodiments shown in FIGS. 77 and 78, the barbed strips 7902 may include barbs 7910 on some portions, and no barbs 7910 in portions extending across the base membrane 7905. The portions of the barbed strips 7902 with barbs 7910 may extend outward from the base membrane 7905, as shown. By this arrangement, in some aspects, none of the barbs 790 may penetrate through the base membrane 7905.

a. Use of Tissue Repair Device Having Barbed Strips Integrated with Base Membrane

FIG. 80 is a schematic view of the tissue repair device 7900, including the two barbed strips 7902 integrated with the base membrane 7905, shown in FIG. 79, in use during a tissue repair process, according to one embodiment. In particular, FIG. 80 shows two barbed strips 7902 with barbs 7910, the integrated base membrane 7905, and two transected tissue ends 8000a and 8000b. The two transected tissue ends 8000a and 8000b may be placed on the base membrane 7905, with the barbed strips 7902 generally aligned with the two transected tissue ends 8000a and 8000b. A portion of the base membrane 7905 and a portion of each of the barbed strips 7902 extending beyond the base membrane 7905 may be positioned on one of the transected tissue ends 8000a and 8000b, and another portion of the base membrane 7905 and another portion of each of the barbed strips 7902 may be generally aligned with the other one of the transected tissue ends 8000a and 8000b. The transected tissue ends 8000a and 8000b may be arranged so that the ends of the transected tissue ends 8000a and 8000b are positioned on the base membrane 7905, and the end regions and central portions of the transected tissue ends 8000a and 8000b are positioned on the barbed strips 7902. The transected tissue ends 8000a and 8000b may be positioned relative to one another so that a gap G₈₀ is formed therebetween, for the reasons discussed above with respect to gap G₆₇. The gap G₈₀ may be approximately 0 mm, or may be greater than approximately 0 mm, such as up to about 5 mm or up to about 10 mm. For example, the gap may be about 0 mm to about 10 mm, about 0 mm to about 8 mm, about 1 mm to about 10 mm, about 1 mm to about 8 mm, about 2 mm to about 8 mm, about 3 mm to about 7 mm, about 3 mm, about 4 mm, about 5 mm, about 6 mm, about 7 mm, etc.

A width W₇₉₀₂ of the barbed strips 7902 may be such that, when the transected tissue ends 8000a and 8000b are placed on the base membrane 7905, the width of each of the barbed strips 7902 lays on or adheres to portions of outer surfaces of the transected tissue ends 8000a and 8000b, as shown in FIG. 80. That is, the barbed strips 7902 may collectively extend around the outer surface of each of the transected tissue ends 8000a and 8000b, and each barbed strip 7902 extends generally in line along the transected tissue ends 8000a and 8000b. The base membrane 7905 may be wrapped around an entire outer surface of each transected tissue end 8000a and 8000b, surrounding or enclosing the gap G₈₀.

Wrapping may include physically moving (e.g., rolling, twisting, or pulling) the base membrane 7905 and laying it on the surface of the transected tissue ends 8000a and 8000b, rolling the transected tissue ends 8000a and 8000b on the base membrane 7905, or a combination thereof. In some aspects, ends of the base membrane 7905 may be secured after wrapping, for example, using a suture, an adhesive, a gel, or capillary action. By this configuration, the barbed strips 7902 and the base membrane 7905 secure the transected tissue ends 8000a and 8000b in place so that a tissue repair process may occur. More specifically, barbs 7910 on the barbed strips 7902 may protrude into or otherwise engage with the outer surface of each of the transected tissue ends 8000a and 8000b, and the barbs 7910 with the base membrane 7905 may inhibit the transected tissue ends 8000a and 8000b from moving apart. In scenarios in which the transected tissue ends 8000a and 8000b are nerve tissue, the base membrane 7905 may also prevent axons that may grow from one or both ends of the tissue ends 8000a and 8000b from escaping, or extending outward, away from the other of the tissue ends 8000a and 8000b.

Although transected tissue ends are discussed in reference to FIGS. 79 and 80, the tissue repair devices of this embodiment may be applied to injured tissues of any type, whether fully transected or not, e.g., for protection or to allow the underlying tissue to heal, or simply to secure tissue for any other suitable reason, unrelated to healing. For example, instead of two tissue ends being brought together, embodiments of the disclosure may wrap around or otherwise cover a portion of tissue that is intact, to promote repair or to otherwise protect the underlying tissue.

### M. Barbed Strips on Strips of a First Base Membrane, and a Second Base Membrane

FIG. 81 is a schematic view of a tissue repair device 8100 including two barbed strips 8102 each adhered to a strip of a first base membrane 8105, integrated with a second base membrane 8110, according to still another embodiment. One or more properties of the first base membrane 8105 and the second base membrane 8110 may be the same as those noted above with respect to base membrane 3900. The barbed strips 8102 may be similar to tissue repair device 7100 described in reference to FIG. 71. The embodiment of FIG. 81 may be similar to, and may be used similarly to, the embodiment of FIG. 79, except that the individual barbed strips 8102 may be adhered or otherwise affixed to strips of base membrane as a backing. In particular, FIG. 81 shows two barbed strips 8102 with barbs 8115, two strips of base membrane 8105 attached to the back of the barbed strips 8102 (i.e., the side opposite from the surface from which the barbs 8115 protrude), and the second base membrane 8110. Similar to the embodiment shown in FIG. 79, the barbed strips 4002 may be formed of a plurality of laterally and longitudinally arranged barbed sutures, or a combination of barbed sutures and non-barbed sutures, or in other words, a plurality of barbed sutures, or a combination of barbed sutures and non-barbed sutures, in a thin grid pattern or mesh, which may be fixed on strips of the first base membrane 8105. Each barbed strip 8102 may include one or more barbs 8115 extending from a surface of the barbed strip 8102 configured to contact with a tissue. The strips of the first base membrane 8105 and barbed strips 8102 may be arranged spaced apart from one or more additional strips of the first base membrane 8105 and barbed strips 8102 on the second base membrane 8110, as described above in reference to FIGS. 77 and 78.

The strips of the first base membrane 8105 with barbed strips 8102 may have a longitudinal length L₈₁₀₅ that is greater than a width W₈₀₁₀ of the second base membrane 8110. Although FIG. 81 shows the tissue repair device 8100 including two strips of the first base membrane 8105 and two barbed strips 8102, a single strip of the first base membrane 8105 and a single barbed strip 8102, or more than two strips of the first base membrane 8105 and more than two barbed strips 8102 may be included. Additionally, the barbed strips 8102 may be located at one or more end regions of the second base membrane 8110, at a central region and at one or more end regions of the second base membrane 8110 (as show in FIG. 81), only at a central region of the second base membrane 8110, or may be spaced in both central and end regions along the second base membrane 8110. Although FIG. 81 depicts the barbed strips 8102 extending generally perpendicular to the second base membrane 8110, with the longest edge of the second base membrane 8110 perpendicular to the length of the barbed strips 8102, other relative orientations of the second base membrane 8110 and the barbed strips 8102 are possible. For example, the barbed strips 8102 may extend generally parallel with the longest edge of the second base membrane 8110, or the barbed strips 8102 may extend at an angle of greater than or less than 90 degrees relative to an edge of the second base membrane 8110. The barbed strips 8102 may be sandwiched between two or more layers of multi-layered second base membranes 8110, as shown in FIG. 81, or may be sewn or passed through holes in the second base membrane 8110, or may be attached in any suitable manner, as described above. And, similar to the embodiments shown in FIGS. 77 and 78, the barbed strips 8102 may include barbs 8115 on some portions, and no barbs 8115 in other portions extending across the second base membrane 8110. The portions of the barbed strips 8102 with barbs 8115 may extend outward from the second base membrane 8110, as shown. By this arrangement, in some aspects, none of the barbs 8115 may penetrate through the second base membrane 8110. During use, the second base membrane 8110 may be applied to tissue in a similar manner as described in regards to FIGS. 77 and 78.

### N. Barbed Mesh and Base Membrane

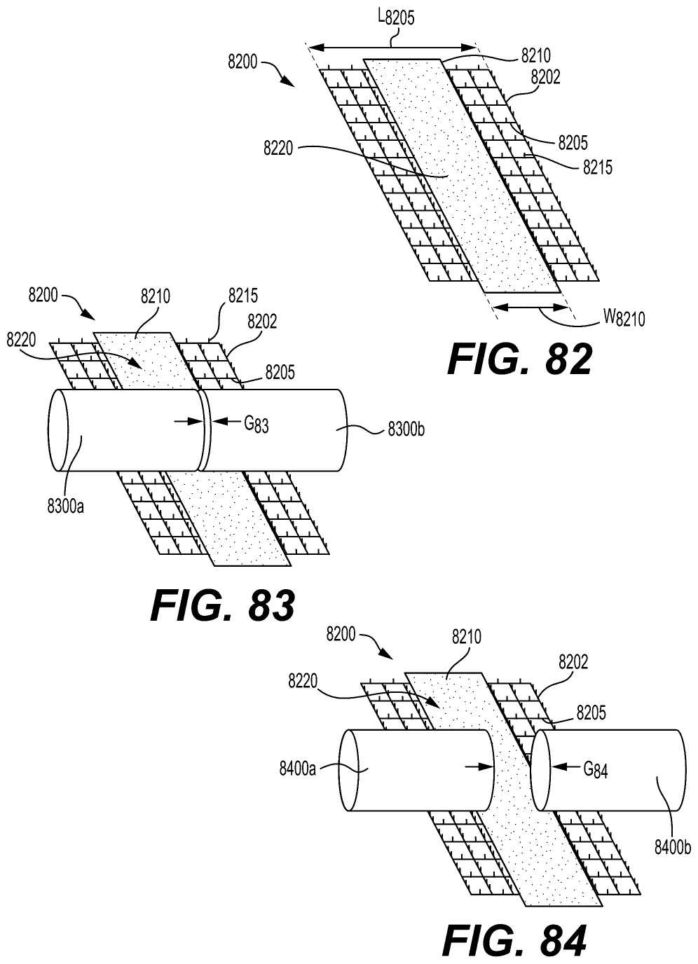

FIG. 82 is a schematic view of a tissue repair device 8200 including a barbed mesh 8202 adhered to or embedded within a base membrane 8210, according to one or more embodiments. One or more properties of the base membrane 8210 may be the same as those noted above with respect to base membrane 3900. In the embodiment of FIG. 82, the barbed mesh 8202 may extend from two sides, e.g., opposite sides, of the base membrane 8210. For example, the base membrane 8210 may extend between portions of the barbed mesh 8202. The mesh 8202 may extend evenly on both sides of the base membrane 8210 or may extend unevenly from both sides of the base membrane 8210. In some aspects, the mesh 8202 may extend along the entirety of two sides of the base membrane 8210, while in other aspects, the mesh 8202 may not extend along the entirety of two sides, or may extend beyond the entirety of two sides of the base membrane 8210.

In some aspects, the mesh 8202 may be formed of a plurality of barbed sutures 8205. Mesh 8202 may include barbed sutures 8205 and barbs 8215, and the base membrane 8210. The barbed mesh 8202 may be formed of a plurality of laterally and longitudinally arranged barbed sutures 8205, or a combination of barbed sutures and non-barbed sutures, in, e.g., a grid pattern. The barbed sutures 8205 may include one or more barbs 8215 extending from a surface of each barbed suture 8205 configured to contact with a tissue. Each barbed suture 8205 may be a separate barbed suture, e.g., woven together or overlaid with the other barbed sutures, or the barbed sutures 8205 may be connected to one another where two or more barbed sutures 8205 intersect, or may be one, uniform mesh instead of woven or overlaid barbed sutures 8205. The barbed sutures 8205 may have a longitudinal length L₈₂₀₅ that is greater than a width W₈₂₁₀ of the base membrane 8210. In embodiments in which the barbed sutures 8205 of the mesh 8202 extend along a surface of the base membrane 8210, the barbed sutures 8205 may include no barbs in locations extending across the base membrane 8210 (in other words, there may be no barbs in the area in which the base membrane 8210 is placed on the mesh 8202 of barbed sutures 8205).

The barbed sutures 8205 may be formed of the materials described herein. The barbed sutures 8205 and the base membrane 8210 may be formed of the same material or they may be formed of different materials, both of which may be, for example, resorbable polymers or metals. For example, if the barbed sutures 8205 and the base membrane 8210 are formed of different resorbable polymers or metals, a barbed mesh 8202 and the base membrane 8210 may have different mechanical properties. For example, the base membrane 8210 may be more flexible, whereas the barbs 8215 may be relatively more rigid in order to extend into and engage with the tissue. In some aspects, the base membrane 8210 may be configured to resorb at a different speed (e.g., faster or slower) compared to the barbs 8215, or at a substantially similar speed.

And, in a case in which the mesh 8202 is attached to the base membrane 8210, the mesh 8202 may be attached to the base membrane 8210 in a suitable manner. For example, tacking or heat welding may be used to adhere the mesh 8202 to the base membrane 8210, as described above. Further, the mesh 8202 may be attached to the base membrane 8210 using a glue or an adhesive. The glue or adhesive may be composed of one or more of the materials described above.

As another example, the mesh 8202 may be attached by weaving or sewing the barbed sutures 8205 through the base membrane 8210, or the barbed sutures 8205 may be hooked onto the base membrane 8210 by making J-shaped or S-shaped cuts in the base membrane 8210 or making small holes in the base membrane 8210, threading the barbed sutures 8205 through the cuts or holes, and tying knots in the ends of the barbed sutures 8205 once threaded through the cuts or holes. As still another example, the barbed sutures 8205 may be attached using heat, such as by melting the barbed sutures 8205 at specific locations as the barbed sutures 8205 lay atop the base membrane 8210.

As still a further example, the barbed sutures 8205 may be attached to the base membrane 8210 using etching, in which barbed sutures 8205 are arranged in a desired orientation in a container, and a material that forms the base membrane 8210 (e.g., a resorbable polymer) is cast around the barbed sutures 8205 in the container, such that the barbed sutures 8205 are completely covered in the material. Then the material may be selectively etched to expose the barbed sutures 8205, on at least one end, and another end of the material may be sliced to cut a thin film, thereby forming the tissue repair device 8200 with the mesh 8202 embedded in the base membrane 8210. A shape and a size of the base membrane 8210 may be determined so that portions of the mesh 8202 with no barbs 8215 lay on the base membrane 8210, as shown in FIG. 82.

a. Use of Tissue Repair Device Having Barbed Mesh and Base Membrane

During use, the base membrane 8210 may be applied to tissue in a similar manner as described in regards to FIGS. 77 to 81. FIG. 83 is a schematic view of the tissue repair device 8200, including the mesh 8202 adhered to the base membrane 8210, as shown in FIG. 82, during a tissue repair process, according to one embodiment. In particular, FIG. 83 shows the barbed mesh 8202, the base membrane 8210, and two transected tissue ends 8300a and 8300b. The two transected tissue ends 8300a and 8300b may be positioned on the barbed mesh 8202 and the base membrane 8210, so that a portion of the barbed mesh 8202 and a portion of the base membrane 8210 are positioned on one of the transected tissue ends 8300a and 8300b, and another portion of the barbed mesh 8202 and another portion of the base membrane 8210 are positioned on the other one of the transected tissue ends 8300a and 8300b. The transected tissue ends 8300a and 8300b may be arranged so that the end regions of the transected tissue ends 8300a and 8300b are positioned on the base membrane 8210, and portions of the transected tissue ends 8300a and 8300b set back from the end regions are positioned on the barbed mesh 8202. The transected tissue ends 8300a and 8300b may be positioned relative to one another so that a gap G₈₃ formed between the transected tissue ends 8300a and 8300b is approximately 0 mm or is less than about 5 mm. Then, the barbed mesh 8202 and the base membrane 8210 may be wrapped around an outer surface of each of the transected tissue ends 8300a and 8300b.

Wrapping the barbed mesh 8202 and the base membrane 8210 may include physically moving (e.g., rolling, twisting, or pulling) the barbed mesh 8202 and the base membrane 8210, and laying them on the surface of the transected tissue ends 8300a and 8300b, rolling the transected tissue ends 8300a and 8300b on the barbed mesh 8202 and the base membrane 8210, or a combination thereof. In some aspects, ends of the barbed mesh 8202, the base membrane 8210, or both, may be secured in place after wrapping, for example, using a suture, an adhesive, a gel, or capillary action. Alternatively, engagement of the barbed mesh 8202 with the tissue may be sufficient to secure the barbed mesh 8202 and the base membrane 8210 in place. By this configuration, the barbed mesh 8202 and the base membrane 8210 may secure the transected tissue ends 8300a and 8300b together so that a tissue repair process may occur. More specifically, barbs 8215 on the barbed sutures 8205 of the barbed mesh 8202 may protrude into or otherwise engage with the outer surface of each of the transected tissue ends 8300a and 8300b, and the barbs 8215, together with the mesh 8202 and the base membrane 8210, may inhibit the transected tissue ends 8300a and 8300b from moving apart.

FIG. 84 is a schematic view of the tissue repair device 8200, as shown in FIG. 82, during a tissue repair process, according to another embodiment. In particular, FIG. 84 shows the mesh 8202, the base membrane 8210, and two transected tissue ends 8400a and 8400b. Similar to the configuration shown in FIG. 83, in this configuration, two transected tissue ends 8400a and 8400b may be positioned on the mesh 8202 of barbed sutures 8205 and the base membrane 8210, but in this configuration, the transected tissue ends 8400a and 8400b may be arranged so that the end regions of the transected tissue ends 8400a and 8400b are positioned on the base membrane 8210, and portions of the transected tissue ends 8400a and 8400b more proximal to the end regions are positioned on the barbed mesh 8202. The transected tissue ends 8400a and 8400b may be positioned relative to one another so that a gap G₈₄ is formed therebetween, for the reasons discussed above with respect to gap G₆₇. The gap G₈₄ may be greater than approximately 0 mm, such as up to about 10 mm or up to about 5 mm. For example, the gap G₈₄ may be about 0 mm to about 10 mm, about 0 mm to about 8 mm, about 1 mm to about 10 mm, about 1 mm to about 8 mm, about 2 mm to about 8 mm, about 3 mm to about 7 mm, about 3 mm, about 4 mm, about 5 mm, about 6 mm, about 7 mm, etc. The wrapping and securing of the mesh 8202 and the base membrane 8210 of this configuration may be the same as that shown and described with respect to FIG. 83.

Although transected tissue ends are discussed in reference to FIGS. 82-84, the tissue repair devices of this embodiment may be applied to injured tissues of any type, whether fully transected or not, e.g., for protection or to allow the underlying tissue to heal, or simply to secure tissue for any other suitable reason, unrelated to healing. For example, instead of two tissue ends being brought together, embodiments of the disclosure may wrap around or otherwise cover a portion of tissue that is intact, to promote repair or to otherwise protect the underlying tissue.

O. Mesh within Strip of Base Membrane

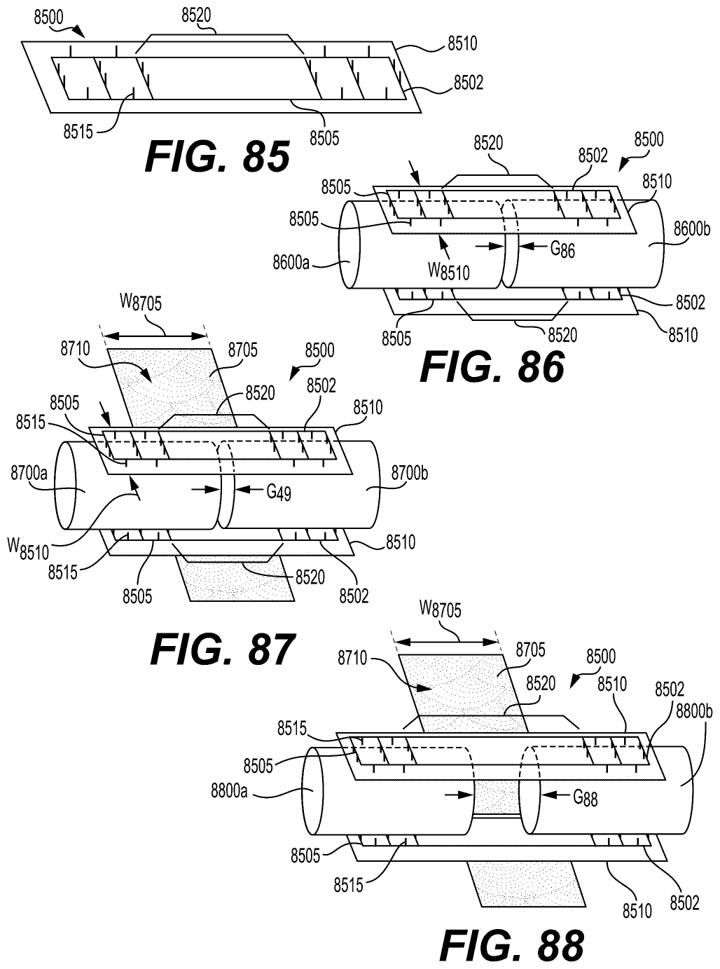

FIG. 85 is a schematic view of a tissue repair device 8500, including a mesh 8502 adhered to or embedded within a strip of base membrane 8510, according to one embodiment. One or more properties of the base membrane 8510 may be the same as those noted above with respect to base membrane 3900. FIG. 85 shows the mesh 8502, which may include barbs 8515 and a portion 8520 free from barbs. The mesh 8502 may be, for example, 3D printed, or may be formed of a plurality of laterally and longitudinally arranged barbed sutures 8505, or a combination of barbed sutures and non-barbed sutures, or, in other words, a plurality of barbed sutures 8505, or a combination of barbed sutures and non-barbed sutures, arranged in a grid pattern. Each of the barbed sutures 8505 may include one or more barbs 8515 extending from a surface of the barbed suture 8505 configured to contact with a tissue, and, as noted above and shown in FIG. 82, the mesh 8502 may include the portion 8520 that is free from barbs 8515. The portion 8520 free from barbs 8515 may be in a central region of the mesh 8502, as shown, however, the portion 8520 free from barbs 8515 may be located at one or both ends of the mesh 8502 in other embodiments.

The mesh 8502 may be attached to the strip of base membrane 8510 using a glue or an adhesive, or in any of the other ways described above. As another example, the mesh 8502 may be attached by weaving or sewing the barbed sutures 8505 through the strip of base membrane 8510, or the barbed sutures 8505 may be hooked onto the strip of base membrane 8510 by making J-shaped or S-shaped cuts in the base membrane 8510 or making small holes in the side of the base membrane 8510, threading the barbed sutures 8505 through the cuts or holes, and tying knots in the ends of the barbed sutures once threaded through the cuts or holes. As still another example, the barbed sutures may be attached using heat, such as by melting the sutures 8505 at specific locations as the barbed sutures 8505 lay atop the strip of base membrane 8510. As still a further example, the barbed sutures 8505 may be attached to the base membrane 8510 using etching, in which barbed sutures 8505 are arranged in a desired orientation in a container, and a material that forms the base membrane 8510 (e.g., a resorbable polymer) is cast around the barbed sutures in the container, such that the barbed sutures 8505 are completely covered in the material. Then the material may be selectively etched to expose the barbed sutures 8505, on at least one end, and another end of the material may be sliced to cut a thin film, thereby forming the tissue repair device 8500 with the mesh 8502 embedded in the strip of base membrane 8510.

a. Use of Tissue Repair Device Having Mesh within Strip of Base Membrane

FIG. 86 is a schematic view of the tissue repair device 8500, during a tissue repair process, according to one embodiment. In particular, FIG. 86 shows two tissue repair devices 8500 and two transected tissue ends 8600a and 8600b. The two tissue repair devices 8500 may be aligned on the transected tissue ends 8600a and 8600b so that a portion of each strip of base membrane 8510 and a portion of each mesh 8502 with barbs 8515 are positioned on one of the transected tissue ends 8600a and 8600b, and another portion of each strip of base membrane 8510 and another portion of each mesh 8502 with barbs 8515 are positioned on the other one of the transected tissue ends 8600a and 8600b. The transected tissue ends 8600a and 8600b may be arranged so that the end regions, set back from the extreme ends, of the transected tissue ends 8600a and 8600b are positioned on the meshes 8502, and the ends of the transected tissue ends 8600a and 8600b are positioned on the strips of base membrane 8510. The transected tissue ends 8600a and 8600b may be positioned relative to one another so that a gap G₈₆ formed between the transected tissue ends 8600a and 8600b may be approximately 0 mm or may be less than about 5 mm. In the embodiment shown in FIG. 86, the portion 8520 of the mesh 8502 that is free from barbs 8515 may be located across the gap G₈₆ between the transected tissue ends 8600a and 8600b.

A width W₈₅₁₀ of the strips of base membrane 8510 with the meshes 8502 may be such that, when the transected tissue ends 8600a and 8600b are placed on the strips of base membrane 8510 and the meshes 8502 (or when the strips of base membrane 8510 with the meshes 8502 are placed on the transected tissue ends 8600a and 8600b), the entire width W₈₅₁₀ of each of the strips of base membrane 8510 lays on or adheres to outer surfaces of the transected tissue ends 8600a and 8600b, as shown in FIG. 86. That is, the strips of base membrane 8510 may extend around a portion of the surface of the transected tissue ends 8600a and 8600b to ‘tape’ the transected tissue ends 8600a and 8600b together. By this configuration, the strips of base membrane 8510 and the meshes 8502 may secure the transected tissue ends 8600a and 8600b together so that a tissue repair process may occur. More specifically, barbs 8515 of the mesh 8502 may protrude into or otherwise engage with the outer surface of each of the transected tissue ends 8600a and 8600b, and the barbs 8515, together with the strips of base membrane 8510, may inhibit the transected tissue ends 8600a and 8600b from moving apart. Although two tissue repair devices 8500 are depicted in FIGS. 86 to 88, one or more than two tissue repair devices 8500 may be used during a procedure.

FIG. 87 is a schematic view of two tissue repair devices 8500, as shown in FIG. 85, and a separate base membrane 8705, during a tissue repair process, according to one embodiment. In particular, FIG. 87 shows strips of base membrane 8510 with barbed meshes 8502, each including barbs 8515 and a portion 8520 free from barbs 8515, the separate base membrane 8705, as well as two transected tissue ends 8700a and 8700b onto which they are applied. The tissue repair devices 8500 may be applied to the two transected tissue ends 8700a and 8700b as described in reference to FIG. 86, but in this embodiment, the separate base membrane 8705 is wrapped on top of at least the central portion of the tissue repair devices 8500. When positioned in place, the separate base membrane 8705 may cover a gap G₈₇ between the transected tissue ends 8700a and 8700b, and may cover at least a portion of the portion 8520 free from barbs 8515. The transected tissue ends 8700a and 8700b may be arranged so that a gap G₄₉ between the transected tissue ends 8700a and 8700b may be approximately 0 mm or may be less than about 5 mm. A shape and a size of the separate base membrane 8705 may be determined so that portions 8520 of the tissue repair device 8500 free from barbs 8515 lay on the separate base membrane 8705, as shown in FIG. 87. During use, the separate base membrane 8705 may be applied to tissue in a similar manner as described in regards to FIGS. 73 and 74.

FIG. 88 is a schematic view of two tissue repair devices 8500, as shown in FIG. 85, and the separate base membrane 8705, shown in FIG. 87, during a tissue repair process, according to another embodiment. In particular, FIG. 88 shows the strips of base membrane 8510 with the meshes 8502, the separate base membrane 8705, and two transected tissue ends 8800a and 8800b. The tissue repair devices 8500 may be applied as described in reference to FIG. 86, and the separate base membrane 8705 may be applied as described in reference to FIG. 87, but, in the embodiment of FIG. 88, the transected tissue ends 8800a and 8800b may be positioned relative to one another so that a gap G₈₈ is formed therebetween, for the reasons discussed above with respect to gap G₆₇. The gap G₈₈ may be greater than approximately 0 mm, up to about 10 mm, or up to about 5 mm. For example, the gap G₈₈ may be about 0 mm to about 10 mm, about 0 mm to about 8 mm, about 1 mm to about 10 mm, about 1 mm to about 8 mm, about 2 mm to about 8 mm, about 3 mm to about 7 mm, about 3 mm, about 4 mm, about 5 mm, about 6 mm, about 7 mm, etc. The wrapping and securing of the separate base membrane 8705 of this configuration may be the same as that shown and described with respect to FIG. 87. If the transected tissue ends 8800a and 8800b are nerve ends, then by covering the transected tissue ends 8800a and 8800b held together by the strips of base membrane 8510 with the meshes 8502 with the separate base membrane 8705, it may be possible to prevent axons that may grow from one or both of the transected tissue ends 8800a and 8800b from escaping, that is, from extending outward and away from the other one of the transected tissue ends 8800a and 8800b and outward from the gap G₈₈.

Although transected tissue ends are discussed in reference to FIGS. 85-88, the tissue repair devices of this embodiment may be applied to injured tissues of any type, whether fully transected or not, e.g., for protection or to allow the underlying tissue to heal, or simply to secure tissue for any other suitable reason, unrelated to healing. For example, instead of two tissue ends being brought together, embodiments of the disclosure may wrap around or otherwise cover a portion of tissue that is intact, to promote repair or to otherwise protect the underlying tissue.

P. Strip of Base Membrane with Barbed Mesh

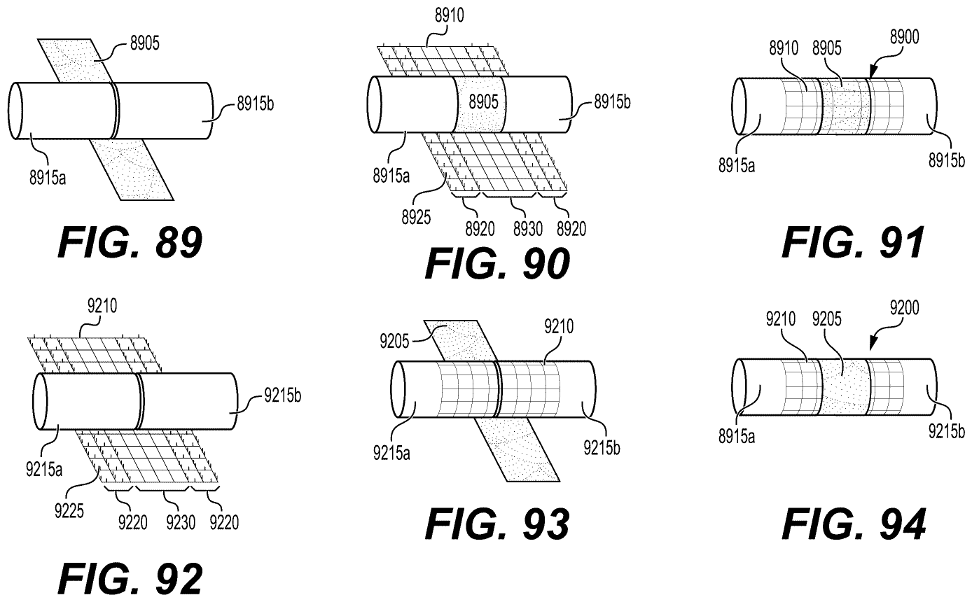

FIGS. 89 to 91 are schematic views of a tissue repair device 8900 formed of a separate strip of base membrane 8905 and a barbed mesh 8910, according to another embodiment. The base membrane 8905 may have one or more properties of the base membrane 3900 described above. FIG. 89 shows the strip of base membrane 8905, with two tissue end 8915a and 8915b laid thereon. The strip of base membrane 8905 may be initially wrapped around the tissue ends 8915a and 8915b. FIG. 90 shows tissue ends 8915a and 8915b, with the strip of base membrane 8905 wrapped around them, placed upon the barbed mesh 8910. The barbed mesh 8910 may have barbed portions 8920, with barbs 8925, and a non-barbed portion 8930 without barbs. The non-barbed portion 8930 may be aligned with the strip of base membrane 8905, as shown. FIG. 91 shows the strip of base membrane 8905 and the barbed mesh 8910 both wrapped around the tissue ends 8915a and 8915b. The barbed mesh 8910 may be wrapped around the tissue ends 8915a and 8915b, as well as the strip of base membrane 8905, to complete the coaptation of the tissue ends 8915a and 8915b. The barbed portions 8920 of the barbed mesh 8910 may affix to the tissue ends 8915a and 8915b only, without physically constraining the wrapped base membrane 8905.

FIGS. 92, 93, and 94 show another embodiment of a tissue repair device 9200, formed of a strip of base membrane 9205 and a barbed mesh 9210, according to another embodiment. The strip of base membrane 9205 may have one or more properties of the base membrane 3900 described above. FIG. 92 shows the barbed mesh 9210, with two tissue ends 9215a and 9215b laid thereon. The barbed mesh 9210 may have barbed portions 9220, with barbs 9225, and a non-barbed portion 9230 without barbs. The barbed mesh 9210 may be initially wrapped around the tissue ends 9215a and 9215b, securing the tissue ends 9215a and 9215b together. FIG. 93 shows tissue ends 9215a and 9215b, with the barbed mesh 9210 wrapped around them, placed upon the strip of base membrane 9205. FIG. 94 shows the strip of base membrane 9205 and the barbed mesh 9210 both wrapped around the tissue ends 9215a and 9215b. The strip of base membrane 9205 may be wrapped around the tissue ends 9215a and 9215b, as well as the barbed mesh 9210, to complete the coaptation of the tissue ends 9215a and 9215b. The barbed portions 9220 of the barbed mesh 9210 may affix to the tissue ends 9215a and 9215b, achieving coaptation, and the strip of base membrane 9205 is wrapped around the coapted tissue ends 9215a and 9215b.

Although transected tissue ends are discussed in reference to FIGS. 89-94, the tissue repair devices of these embodiments may be applied to injured tissues of any type, whether fully transected or not, e.g., for protection or to allow the underlying tissue to heal, or simply to secure tissue for any other suitable reason, unrelated to healing. For example, instead of two tissue ends being brought together, embodiments of the disclosure may wrap around or otherwise cover a portion of tissue that is intact, to promote repair or to otherwise protect the underlying tissue.

Q. Base Membrane Having Tab with Barbed Sutures

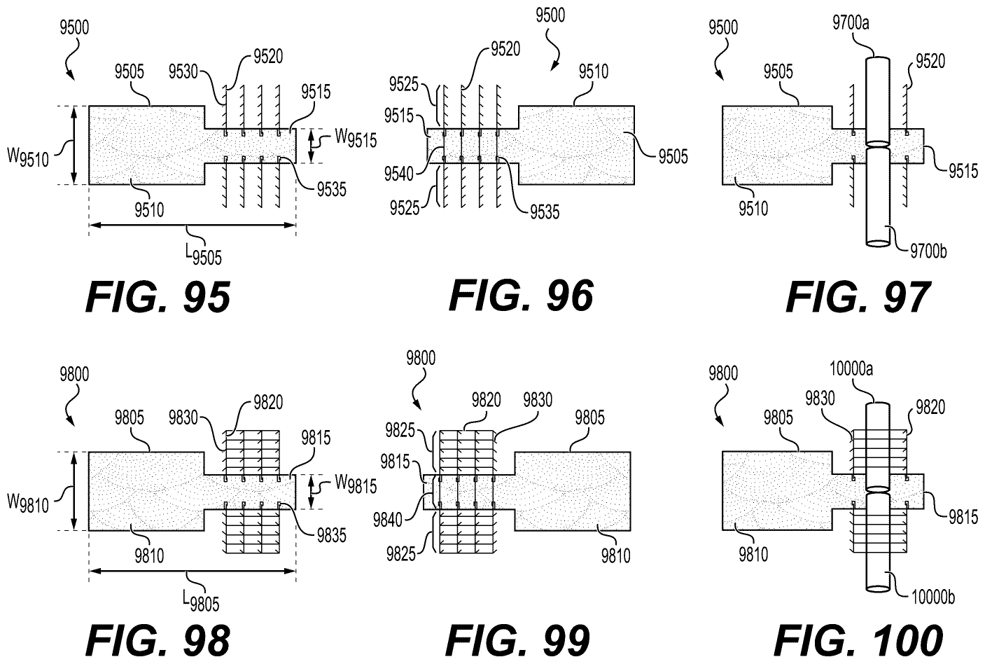

FIGS. 95 to 97 show another embodiment of a tissue repair device 9500, formed of a base membrane 9505 having a large tab 9510 and a small tab 9515, forming a relatively narrow portion of the base membrane 9505, and a plurality of barbed sutures 9520. The base membrane 9505 may have one or more properties of the base membrane 3900 described above. The barbed sutures 9520 may be bi-directional barbed sutures, in that they include a plurality of barbs 9530 pointing in one direction, and a plurality of barbs 9530 pointing in another direction.

FIG. 95 shows the base membrane 9505, with the large tab 9510 and the small tab 9515. In one example embodiment, a width W₉₅₁₀ of the large tab 9510 may be about 2 cm to about 2.5 cm, and a width W₉₅₁₅ of the small tab 9510 may be about 1 cm to about 1.5 cm, although any suitable wider and relatively narrower widths may be used based on the intended use of the tissue repair device 9500. An overall length L₉₅₀₅ of the base membrane 9505 may be based on a size or diameter of tissue to be repaired using the tissue repair device 9500.

The barbed sutures 9520 may be attached to the small tab 9515, as shown in FIG. 95, by making cuts 9535 in edges of the small tab 9515, and hooking the barbed sutures 9520 into the cuts 9535, as described above with respect to FIG. 48. FIG. 96 shows a back side of the tissue repair device 9500, and in particular, shows barbed portions 9525 of the barbed sutures 9520, having barbs 9530, and non-barbed portions 9540 of the barbed sutures 9520 having no barbs. As in the embodiment described above with respect to FIG. 48, the barbed sutures 9520 may be hooked onto the small tab 9515 such that the barbed portions 9525 extend outward from the small tab 9515, and the non-barbed portions 9540 overlap with the small tab 9515, as shown. That said, the barbed sutures 9520 may be attached to the small tab 9515 in any suitable manner as described above, e.g., adhesive, sewing, weaving, heating, etc.

FIG. 97 shows two tissue ends 9700a and 9700b arranged longitudinally, aligning with the barbed sutures 9520 and overlapping a least one barbed suture 9520, and the small tab 9515 of the base membrane 9505 positioned relative to the tissue ends 9700a and 9700b. The tissue repair device 9500 may be wrapped around the tissue ends 9700a and 9700b, such that the small tab 9515, with the barbed sutures 9520, wrap around the tissue ends 9700a and 9700b, with the barbs 9530 of the barbed sutures 9520 engaging with the tissue ends 9700a and 9700b, achieving coaptation, and the large tab 9510 also wrapping around the tissue ends 9700a and 9700b to further provide mechanical stability, to minimize the risk for axonal escape if used with nerves, and to protect a larger area of the tissue ends 9700a and 9700b around the coaptation site.

The small tab 9515 having the barbed sutures 9520 helps to bring the bidirectional barbs 9530 closer to a site of coaptation between tissue ends 9700a and 9700b, to help maintain alignment of the tissue ends 9700a and 9700b. As an example, in a case in which tissue ends 9700a and 9700b are nerve tissue ends, the small tab 9515 with the bidirectional barbs 9530 helps to maintain fascicular aligning, that is, alignment between fascicles within the nerve tissue ends. The large tab 9510 then wraps around the small tab and provides mechanical stability, minimizes the risk for axonal escape, in a case in which the tissue ends 9700a and 9700b are nerve tissue ends, and protects a larger area around the coaptation site.

Although transected tissue ends are discussed in reference to FIGS. 95-97, the tissue repair devices of this embodiment may be applied to injured tissues of any type, whether fully transected or not, e.g., for protection or to allow the underlying tissue to heal, or simply to secure tissue for any other suitable reason, unrelated to healing. For example, instead of two tissue ends being brought together, embodiments of the disclosure may wrap around or otherwise cover a portion of tissue that is intact, to promote repair or to otherwise protect the underlying tissue.

R. Base Membrane Having Tab with Barbed Mesh

FIGS. 98 to 100 show another embodiment of a tissue repair device 9800, formed of a base membrane 9805 having a large tab 9810 and a small tab 9815, and a barbed mesh 9820. The base membrane 9805 may have one or more properties of the base membrane 3900 described above. The barbed mesh 9820 may be formed of a plurality of bi-directional barbed sutures, in that they include a plurality of barbs 9830 pointing in one direction, and a plurality of barbs 9830 pointing in another direction.

FIG. 98 shows the base membrane 9805, with the large tab 9810 and the small tab 9815. In one example embodiment, a width W₉₈₁₀ of the large tab 9810 may be about 2 cm to about 2.5 cm, and a width W₉₈₁₅ of the small tab 9810 may be about 1 cm to about 1.5 cm, although any suitable wider and relatively narrower widths may be used based on the intended use of the tissue repair device 9800. An overall length L₉₈₀₅ of the base membrane 9805 may be based on a size or diameter of tissue to be repaired using the tissue repair device 9800.

The barbed mesh 9820 may be attached to the small tab 9815, as shown in FIG. 98, by making cuts 9835 in edges of the small tab 9815, and hooking the barbed mesh 9820 into the cuts 9835, as described above with respect to FIG. 48. FIG. 99 shows a back side of the tissue repair device 9800, and in particular, shows barbed portions 9825 of the barbed mesh 9820, having barbs 9830, and non-barbed portions 9840 of the barbed mesh 9820 having no barbs. As in the embodiment described above with respect to FIG. 48, the barbed mesh 9820 may be hooked onto the small tab 9815 such that the barbed portions 9825 extend outward from the small tab 9815, and the non-barbed portions 9840 overlap with the small tab 9815, as shown. That said, the barbed mesh 9820 may be attached to the small tab 9815 in any suitable manner as described above, e.g., adhesive, sewing, weaving, heating, etc.

FIG. 100 shows two tissue ends 10000a and 10000b arranged longitudinally, aligning with longitudinal portions of the barbed mesh 9820, and overlapping a least a portion of the barbed mesh 9820, and the small tab 9815 of the base membrane 9805 positioned relative to the tissue ends 10000a and 10000b. The tissue repair device 9800 may be wrapped around the tissue ends 10000a and 10000b, such that the small tab 9815, with the barbed mesh 9820 hooked into the small tab 9815, wrap around the tissue ends 10000a and 10000b, with the barbs 9830 of the barbed mesh 9820 engaging with the tissue ends 10000a and 10000b, achieving coaptation, and the large tab 9810 also wrapping around the tissue ends 10000a and 10000b to further provide mechanical stability, to minimize the risk for axonal escape, and to protect a larger area of the tissue ends 10000a and 10000b around the coaptation site, as described above with reference to FIG. 97.

As in the embodiment of FIGS. 95 to 97, the small tab 9815 having the barbed mesh 9820 helps to bring the bidirectional barbs 9830 closer to a site of coaptation between tissue ends 10000a and 10000b, to help maintain alignment of the tissue ends 10000a and 10000b. The use of the barbed mesh 9820 further helps to inhibit misalignment of barbed sutures and to ensure the barbs 9830 on the barbed sutures always face and engage with tissue ends 10000a and 10000b. As an example, in a case in which tissue ends 10000a and 10000b are nerve tissue ends, the small tab 9815 with the bidirectional barbs 9830 helps to maintain fascicular aligning, that is, alignment between fascicles within the nerve tissue ends. The large tab 9810 provides mechanical stability, minimizes the risk for axonal escape, in a case in which the tissue ends 10000a and 10000b are nerve tissue ends, and protects a larger area around the coaptation site.

Although transected tissue ends are discussed in reference to FIGS. 98-100, the tissue repair devices of this embodiment may be applied to injured tissues of any type, whether fully transected or not, e.g., for protection or to allow the underlying tissue to heal, or simply to secure tissue for any other suitable reason, unrelated to healing. For example, instead of two tissue ends being brought together, embodiments of the disclosure may wrap around or otherwise cover a portion of tissue that is intact, to promote repair or to otherwise protect the underlying tissue.

### S. Base Membrane Having Barbed Sutures

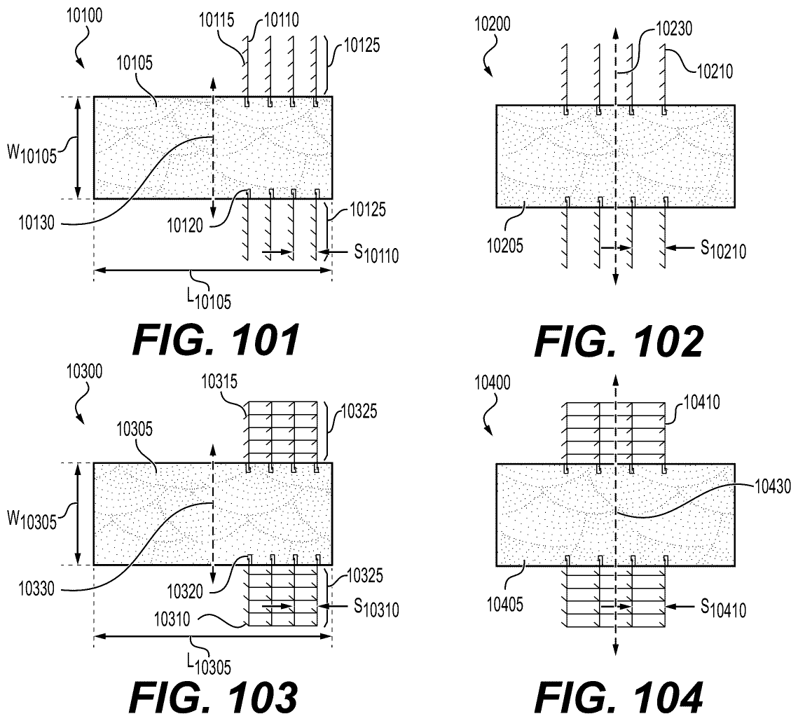

FIG. 101 shows another embodiment of a tissue repair device 10100, formed of a base membrane 10105 and a plurality of barbed sutures 10110. The base membrane 10105 may have one or more properties of the base membrane 3900 described above. The barbed sutures 10110 may be bi-directional barbed sutures, in that they include a plurality of barbs 10115 pointing in one direction, and a plurality of barbs 10115 pointing in another direction.

The base membrane 10105 may have a width W₁₀₁₀₅ of about 2 cm to about 2.5 cm, and a length L₁₀₁₀₅ may be based on a size or diameter of tissue to be repaired using the tissue repair device 10100, although any suitable lengths and widths may be used. The barbed sutures 10110 may be attached to the base membrane 10105, as shown in FIG. 101, by making cuts 10120 in edges of the base membrane 10105, and hooking the barbed sutures 10110 into the cuts 10120, as described above with respect to FIG. 48. Although this attachment mechanism is shown, any suitable attachment methodology described herein may be used. The barbed sutures 10110 may be attached on one side of the base membrane 10105, that is, they may all be located on one side of a center line 10130 of the base membrane 10105. The barbed sutures 10110 may also be arranged at a predetermined spacing S₁₀₁₁₀, which may be determined based on the type of tissue to be repaired using the tissue repair device 10100 or the type of repair procedure. The barbed sutures 10110 may have barbed portions 10125, having barbs 10115, and a non-barbed portion (not shown), having no barbs 10115. As in the embodiment described above with respect to FIG. 48, the barbed sutures 10110 may be hooked onto the base membrane 10105 such that barbed portions 10125 extend outward from the base membrane 10105, and the non-barbed portions overlap with the base membrane 10105.

By virtue of the base membrane 10105 having a rectangular shape, as shown in FIG. 101, without a small tab, the tissue repair device 10100 may be relatively easier or simpler to manufacture compared to the embodiment of FIGS. 95-97, although they may function in a similar manner.

FIG. 102 shows another embodiment of a tissue repair device 10200, formed of a base membrane 10205 and a plurality of barbed sutures 10210. The tissue repair device 10200 may be the same as the tissue repair device 10100 shown in FIG. 101, but differs in that the barbed sutures 10210 may be attached centrally on the base membrane 10205, that is, they may be symmetrically arranged about a center line 10230 of the base membrane 10205, and the barbed sutures 10210 may be arranged at a relatively larger predetermined spacing S₁₀₂₁₀, as compared to spacing S₁₀₁₁₀, although any suitable spacing may be used.

As with the embodiment of FIG. 101, by virtue of the base membrane 10205 having a rectangular shape, as shown in FIG. 102, without a small tab, the tissue repair device 10200 may be relatively easier or simpler to manufacture and may function in a similar manner.

### T. Base Membrane Having Barbed Mesh

FIG. 103 shows another embodiment of a tissue repair device 10300, formed of a base membrane 10305 and a barbed mesh 10310. The base membrane 10305 may have one or more properties of the base membrane 3900 described above. The barbed mesh 10310 may be formed of a plurality of bi-directional barbed sutures, each including a plurality of barbs 10315 pointing in one direction, and a plurality of barbs 10315 pointing in another direction.

The base membrane 10305 may have a width W₁₀₃₀₅ of about 2 cm to about 2.5 cm, and a length L₁₀₃₀₅ may be based on a size or diameter of tissue to be repaired using the tissue repair device 10300, although any suitable lengths and widths may be used. The barbed mesh 10310 may be attached to the base membrane 10305, as shown in FIG. 103, by making cuts 10320 in edges of the base membrane 10305, and hooking the barbed mesh 10310 into the cuts 10320, as described above with respect to FIG. 48. Although this attachment mechanism is shown, any suitable attachment methodology described herein may be used. The barbed mesh 10310 may be attached on one side of the base membrane 10305, that is, the barbed mesh 10310 be located on one side of a center line 10330 of the base membrane 10305. The barbed mesh 10310 may also have a predetermined spacing S₁₀₃₁₀ between longitudinal sutures forming the mesh 10310, which may be determined based on the type of tissue to be repaired using the tissue repair device 10300, or the type of repair procedure. The barbed mesh 10310 may have barbed portions 10325, having barbs 10315, and a non-barbed portion (not shown), having no barbs 10315. As in the embodiment described above with respect to FIG. 48, the barbed mesh 10310 may be hooked onto the base membrane 10305 such that barbed portions 10325 extend outward from the base membrane 10305, and the non-barbed portion overlaps with the base membrane 10305.

By virtue of the base membrane 10305 having a rectangular shape, as shown in FIG. 103, without a small tab, the tissue repair device 10300 may be relatively easier or simpler to manufacture and may function in a similar manner.

FIG. 104 shows another embodiment of a tissue repair device 10400, formed of a base membrane 10405 and a barbed mesh 10410. The tissue repair device 10400 may be the same as the tissue repair device 10300 shown in FIG. 103, but differs in that the barbed mesh 10410 may be attached centrally on the base membrane 10405, that is, the barbed mesh 10410 be centered on a center line 10430 of the base membrane 10405, and a predetermined spacing S₁₀₄₁₀ between longitudinal sutures forming the mesh 10410, as compared to spacing S₁₀₃₁₀, although any suitable spacing may be used.

As with the embodiment of FIG. 103, by virtue of the base membrane 10405 having a rectangular shape, as shown in FIG. 104, without a small tab, the tissue repair device 10400 may be relatively easier or simpler to manufacture and may be used in a similar manner.

U. Base Membrane with Longitudinally Arranged Barbed Sutures

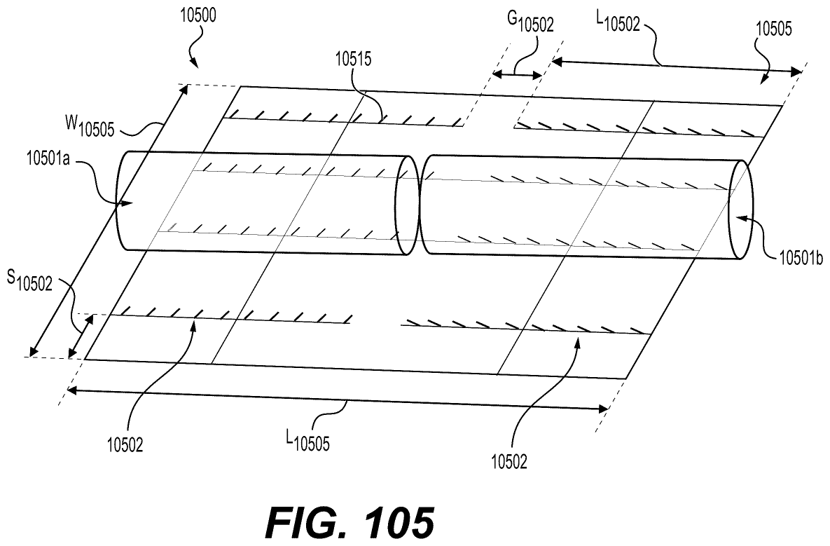

FIG. 105 is a schematic view of a tissue repair device 10500 including a plurality of barbed sutures 10502 mounted on or embedded within a base membrane 10505, according to another embodiment. One or more properties of the base membrane 10505 may be the same as those noted above with respect to base membrane 3900. FIG. 105 also shows two tissue ends 10501a and 10501b, placed on the tissue repair device 10500 during a tissue repair process. In particular, FIG. 105 shows four sets of barbed sutures 10502, with two sets of the barbed sutures 10502 positioned adjacent to the tissue ends 10501a and 10501b, and two sets of the barbed sutures 10502 being under the tissue ends 10501a and 10501b.

A length L₁₀₅₀₅ of base membrane 10505 may be about 35 mm and a width W₁₀₅₀₅ of the base membrane 10505 may be about 18 mm. The barbed sutures 10502 may be parallel to a long side of the base membrane 10505, as shown. A length L₁₀₅₀₂ of the barbed sutures 10502 may be about 15 mm, in a case in which set of barbed sutures 10502 are provided. Alternatively, the length L₁₀₅₀₂ of about 15 mm may be a length of a barbed portion of the barbed suture 10502, and an overall length of the barbed suture 10502 as one continuous longitudinal suture, may be the same as the length L₁₀₅₀₅ of the base membrane 10505. A spacing S₁₀₅₀₂ between adjacent sutures may be about 3 mm. It should be recognized, however, that the example dimensions provided herein are exemplary only and are not limiting, as the specific dimensions may depend, at least in part, on the tissue to which the tissue repair device is to be affixed or the intended use of the tissue repair device.

A gap G₁₀₅₀₂ may be provided between barbed sutures 10502, in a case in which the barbed sutures 10502 are provided as sets, with pairs of barbed sutures being positioned at opposite ends of the base membrane 10505 from each other. Alternatively the barbed sutures 10502 may be long, continuous barbed sutures 10502, extending along the length of the base membrane 10505, with central portions of the barbed sutures 10502 being woven through the base membrane 10505, thereby forming a space between barbed portions of the barbed sutures 10502 on a surface of the base membrane 10505.

The barbed sutures 10502 may be, for example, barbed sutures according to one of the embodiments described herein. Alternatively, the barbed sutures 10502 may be 3D printed. Each barbed suture 10502 may include one or more barbs 10515 extending from a surface of the barbed suture 10502 configured to contact with a tissue. As depicted in FIG. 105, the barbed sutures 10502 may be arranged such that the one or more barbs 10515 of the barbed sutures 10502 are oriented in different directions. The multi-directional arrangement of the one or more barbs 10515 may help the tissue repair device 10500 resist movement or displacement in one or more directions. For example, the multi-directional arrangement of the one or more barbs 10515 may inhibit tissue ends 10501a and 10501b from pulling away from each other.

The barbed sutures 10502 may be mounted to the base membrane 10505 using a glue or an adhesive. The glue or adhesive may include one or more of the materials described above with respect to the embodiment shown in FIGS. 24 to 28.

As another example, the barbed sutures 10502 may be attached by weaving or sewing the barbed sutures 10502 through the base membrane 10505, or the barbed sutures 10502 may be hooked onto the base membrane 10505 by making J-shaped or S-shaped cuts in the base membrane 10505 or making small holes in the side of the base membrane 10505, threading the barbed sutures 10502 through the cuts or holes, and tying knots in the ends of the sutures 10502 once threaded through the cuts or holes. As still another example, the barbed sutures 10502 may be attached using heat, such as by melting the barbed sutures 10502 at specific locations as the barbed sutures 10502 lay atop the base membrane 10505.

As still a further example, the barbed sutures 10502 may be attached to the base membrane 10505 using etching, as described above.

Although transected tissue ends are discussed in reference to FIG. 105, the tissue repair devices of this embodiment may be applied to injured tissues of any type, whether fully transected or not, e.g., for protection or to allow the underlying tissue to heal, or simply to secure tissue for any other suitable reason, unrelated to healing. For example, instead of two tissue ends being brought together, embodiments of the disclosure may wrap around or otherwise cover a portion of tissue that is intact, to promote repair or to otherwise protect the underlying tissue.

V. Strip of Base Membrane with Barbed Sutures

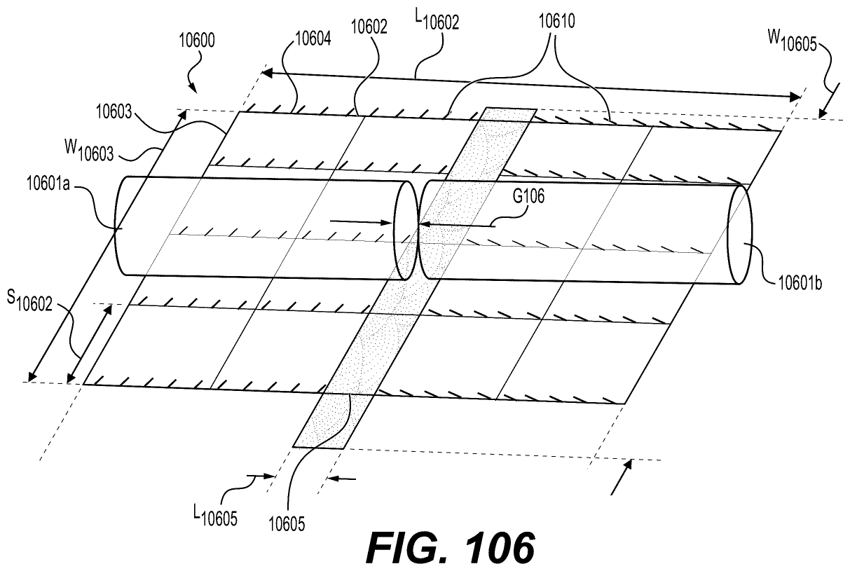

FIG. 106 is a schematic view of a tissue repair device 10600 including barbed sutures 10602, intersecting with non-barbed sutures 10603, as a mesh 10604, integrated with a strip of base membrane 10605, in use during a tissue repair process, according to still another embodiment. One or more properties of the base membrane 10605 may be the same as those noted above with respect to base membrane 3900. FIG. 106 shows a plurality of barbed sutures 10602 with barbs 10610, the non-barbed sutures 10603, the integrated strip of base membrane 10605, and two transected tissue ends 10601a and 10601b. The two transected tissue ends 10601a and 10601b may be placed on the base membrane 10605, with the barbed sutures 10602 being generally aligned with the two transected tissue ends 10601a and 10601b. A portion of the base membrane 10605 and a portion of each of the barbed sutures 10602 which extend beyond the base membrane 10605 may be positioned on one of the transected tissue ends 10601a and 10601b, and another portion of the base membrane 10605 and another portion of each of the barbed sutures 10602 may be generally aligned with the other one of the transected tissue ends 10601a and 10601b. The transected tissue ends 10601a and 10601b may be arranged so that the ends of the transected tissue ends 10601a and 10601b are positioned on the base membrane 10605, and the end regions and central portions of the transected tissue ends 10601a and 10601b are positioned on the barbed sutures 10602. The transected tissue ends 10601a and 10601b may be positioned relative to one another so that a gap G₁₀₆ is formed therebetween, for the reasons discussed above with respect to gap G₆₇. The gap G₁₀₆ may be approximately 0 mm, or may be greater than approximately 0 mm, such as up to about 5 mm or up to about 10 mm. For example, the gap G₁₀₆ may be about 0 mm to about 10 mm, about 0 mm to about 8 mm, about 1 mm to about 10 mm, about 1 mm to about 8 mm, about 2 mm to about 8 mm, about 3 mm to about 7 mm, about 3 mm, about 4 mm, about 5 mm, about 6 mm, about 7 mm, etc.

A length L₁₀₆₀₅ of the strip of base membrane 10605 may be about 5 mm and a width W₁₀₆₀₅ of the base membrane 10605 may be about 18 mm. The barbed sutures 10502 may be perpendicular to a long side of the base membrane 10505, as shown. A length L₁₀₆₀₂ of the barbed sutures 10602 may be about 35 mm. A spacing S₁₀₆₀₂ between adjacent barbed sutures 10502 may be about 3 mm. The non-barbed sutures 10603 may be parallel to the long side of the base membrane 10505, as shown. A width W₁₀₆₀₃ of the non-barbed sutures 10603 may be about 15 mm. It is to be understood, however, that these dimensions are only exemplary, and any suitable dimensions, e.g., based on the intended use of the tissue repair device or the type of tissue to which it will be attached, may be used.

The length L₁₀₆₀₂ of the barbed sutures 10602 and the width W₁₀₆₀₃ of the non-barbed sutures 10603 may be determined such that, when the transected tissue ends 10601a and 10601b are placed on the base membrane 10605, the width of each of the barbed sutures 10602 lays on or adheres to portions of outer surfaces of the transected tissue ends 10601a and 10601b, as shown in FIG. 106. That is, the barbed sutures 10602 may collectively extend around the outer surface of each of the transected tissue ends 10601a and 10601b, and each barbed suture 10602 extends generally in line along the transected tissue ends 10601a and 10601b. The base membrane 10605 may be wrapped around an entire outer surface of each transected tissue end 10601a and 10601b, surrounding or enclosing the gap G₁₀₆. The width W₁₀₆₀₂ in this embodiment may be sized so as to cover the gap G₁₀₆, as well as the ends of the transected tissue ends 10601a and 10601b, but the majority of the transected tissue ends 10601a and 10601b may be engaged by the plurality of barbed sutures 10602.

Wrapping may include physically moving (e.g., rolling, twisting, or pulling) the base membrane 10605 and laying it on the surface of the transected tissue ends 10601a and 10601b, rolling the transected tissue ends 10601a and 10601b on the base membrane 10605, or a combination thereof. In some aspects, ends of the base membrane 10605 may be secured after wrapping, for example, using a suture, an adhesive, a gel, or capillary action. By this configuration, the barbed sutures 10602 and the base membrane 10605 secure the transected tissue ends 10601a and 10601b in place so that a tissue repair process may occur. More specifically, barbs 10610 on the barbed sutures 10602 may protrude into or otherwise engage with the outer surface of each of the transected tissue ends 10601a and 10601b, and the barbs 10610 with the base membrane 10605 may inhibit the transected tissue ends 10601a and 10601b from moving apart. In scenarios in which the transected tissue ends 10601a and 10601b are nerve tissue, the barbs 10610 may engage the nerve epineurium. The base membrane 10605 may also prevent axons that may grow from one or both ends of the tissue ends 10601a and 10601b from escaping, or extending outward, away from the other of the tissue ends 10601a and 10601b.

Although transected tissue ends are discussed in reference to FIG. 106, the tissue repair devices of this embodiment may be applied to injured tissues of any type, whether fully transected or not, e.g., for protection or to allow the underlying tissue to heal, or simply to secure tissue for any other suitable reason, unrelated to healing. For example, instead of two tissue ends being brought together, embodiments of the disclosure may wrap around or otherwise cover a portion of tissue that is intact, to promote repair or to otherwise protect the underlying tissue.

W. Base Membrane with Slits and Longitudinal Barbed Sutures

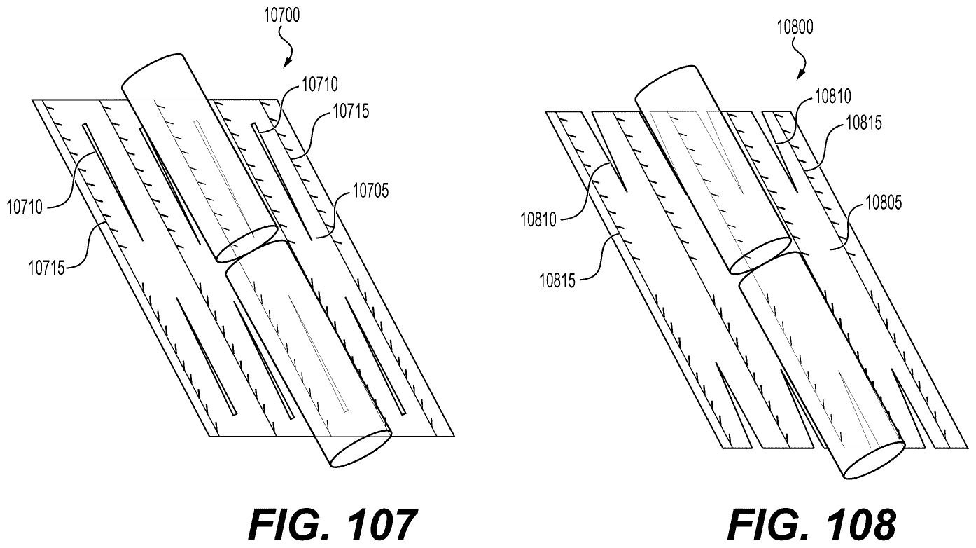

FIG. 107 is a schematic view of a tissue repair device 10700 having a base membrane 10705 with slits or openings 10710 positioned away from one or more edges of the tissue repair device 10700, that is, slits extending within the base membrane 10705, and longitudinally arranged barbed sutures 10715. The tissue repair device 10700 may be similar to the tissue repair device 10500 shown in FIG. 105, but may differ in that the slits 10710 are provided within the base membrane 10705.

FIG. 108 is a schematic view of a tissue repair device 10800 having a base membrane 10805 with slits or openings 10810 located along one or more edges of the base membrane 10805, that is, slits extending towards exterior edges of the base membrane 10805, and longitudinally arranged barbed sutures 10815. The tissue repair device 10800 may be the same as the tissue repair device 10500 shown in FIG. 105, but may differ in that the slits 10810 are provided within the base membrane 10705.

The slits or openings 10710 and 10810 may be included in the base membrane to allow for tissue to which the tissue repair device is applied to swell, contract, flex, rotate, or otherwise move after application of the tissue repair device without impinging, constricting, or putting too much pressure on the tissue. For example, swelling, shrinking, or flexion may occur between the barbed attachment sites in the location of the slits. The base membrane on either side of a given slit may more easily be able to move towards or away from each other, or to splay out or bend, even if there is less give or stretchiness to the material from which the base membrane is formed.

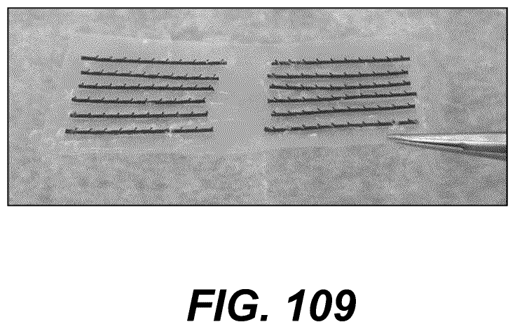

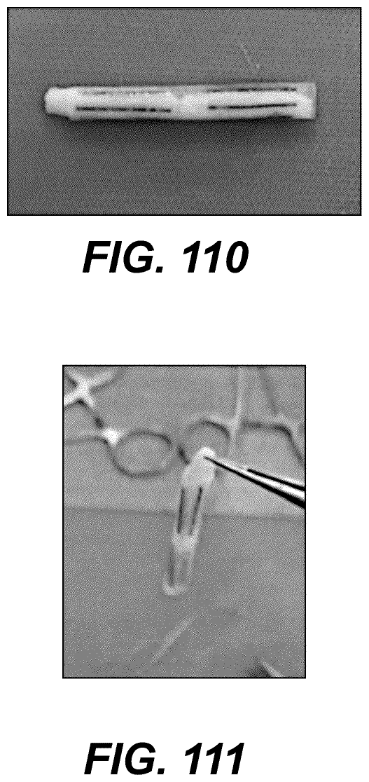

FIGS. 109 to 111 are images of a tissue repair device, as in FIG. 105, with FIG. 109 depicting a base membrane with longitudinally arranged barbed sutures attached thereto. FIG. 110 depicts sample tissue ends, in this case, nerve ends, wrapped in the base membrane and held in place by the barbed sutures, and FIG. 111 depicts the wrapped tissue ends and base membrane and barbed sutures being held, and demonstrates the ability of the tissue repair device to remain in place and engaged with the tissue ends. Although transected tissue ends are discussed in reference to FIGS. 107 to 111, the tissue repair devices of this embodiment may be applied to injured tissues of any type, whether fully transected or not, e.g., for protection or to allow the underlying tissue to heal, or simply to secure tissue for any other suitable reason, unrelated to healing. For example, instead of two tissue ends being brought together, embodiments of the disclosure may wrap around or otherwise cover a portion of tissue that is intact, to promote repair or to otherwise protect the underlying tissue.

X. Base Membrane with Barbed Constructs

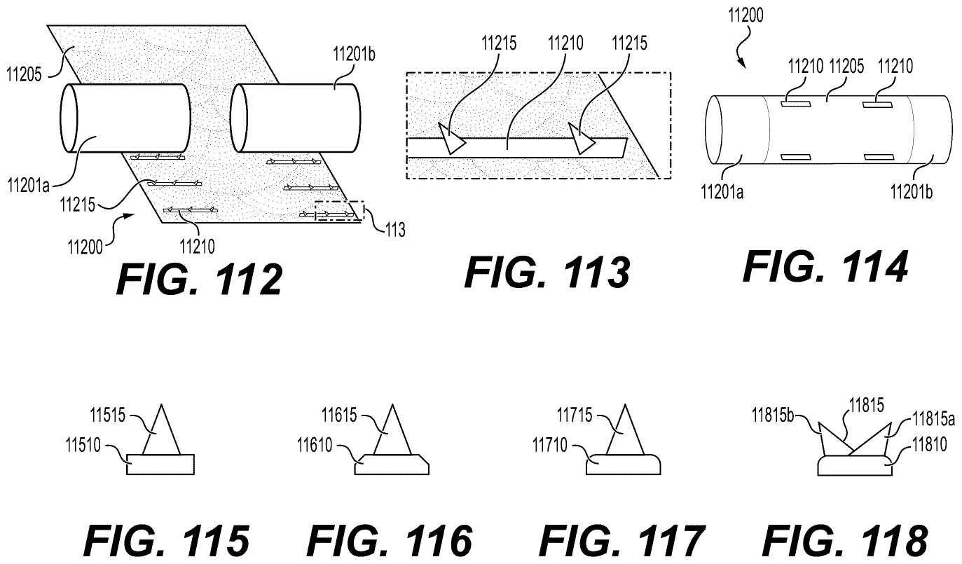

FIG. 112 is a schematic view of a tissue repair device 11200 according to still another embodiment. The tissue repair device 11200 has a base membrane 11205 and barbed constructs 11210 of having barbs 11215. In some aspects, the barbs 11215 may be micro-injection molded. The base membrane 11205 may have one or more of the properties of the base membrane 3900 described above. Each barbed construct 11210 may be formed as a thin substrate, and may be formed of a same material as the barbs 11215. The barbed constructs 11210 may be arranged on the base membrane 11205 in a grid pattern, as shown, with a long edge of each barbed construct 11210 being parallel to tissue ends 11201a and 11201b when placed onto the base membrane. Other arrangements, orientations, or configurations of barbed constructs 11210 may be used, such as perpendicular arrangements, or offset or other arrangements of barbed constructs 11210. In some aspects, the barbed constructs 11210 may be attached along an entire base membrane 11205, or in other aspects, the barbed constructs 11210 may be attached along less than an entire base membrane 11205, for example, along one more edges of base membrane 11205, or along approximately half or less than half of the base membrane 11205.

FIG. 113 is a detail view of an example of micro-injection molded barbs 11215. The thin substrate forming a barbed construct 11210 may be part of a mold used to form the barbs 11215. A polymer may then be added to the mold to thereby form the barbs 11215. Any of the polymers listed herein may be used in forming the barbs 11215, including resorbable polymers.

FIG. 114 depicts the tissue repair device 11200 wrapped around the tissue ends 11201a and 11201b. The barbs of the barbed constructs 11210 engage with surfaces of the tissue ends 11201a and 11201b, achieving coaptation of the tissue ends 11201a and 11201b, while the base membrane 11205 encloses the coapted tissue ends 11201a and 11201b. As in other embodiments, the base membrane 11205 may provide mechanical stability, minimize the risk for axonal escape, and protect a larger area of the tissue ends 10201a and 10201b around the coaptation site.

FIG. 115 shows a side cross-sectional view of a strip 11510 and a barb 11515 formed thereon, according to one embodiment. The strip 11510 may have a rectangular cross-sectional shape, and the barb 11515 may have a triangular cross-sectional shape. FIG. 116 shows a side cross-sectional view of a strip 11610 and a barb 11615 according to another embodiment. The strip 11610 may have a generally rectangular cross-sectional shape, with upper edges or corners of the strip 11610 having a chamfered edge. The barb 11615 may have a triangular cross-sectional shape. FIG. 117 shows a side cross-sectional view of a strip 11710 and a barb 11715 according to still another embodiment. The strip 11710 may have a generally rectangular cross-sectional shape, with upper edges or corners of the strip 11710 having a rounded edge. The barb 11715 may have a triangular shape. FIG. 118 shows a cross-sectional side view of a strip 11810 and a multi-directional barb 11815 according to yet another embodiment. The strip 11810 may have a generally rectangular cross-sectional shape, with upper edges or corners of the strip 11810 having rounded edges. The multi-directional barb 11815 may have the shape of two triangles partially overlapping each other, as shown, such that the barb 11815 has two points 11815a and 11815b. Any one of the cross-sectional shapes of the strips and barbs of FIGS. 115 to 118 may be used in the tissue repair device 11200 described above. Further, formation of the barbed constructs 11210 via micro-injection molding may allow for a variety of different potential cross-sectional shapes, only a sample of which are depicted in FIGS. 115 to 118.

Although transected tissue ends are discussed in reference to FIGS. 112 to 118, the tissue repair devices of this embodiment may be applied to injured tissues of any type, whether fully transected or not, e.g., for protection or to allow the underlying tissue to heal, or simply to secure tissue for any other suitable reason, unrelated to healing. For example, instead of two tissue ends being brought together, embodiments of the disclosure may wrap around or otherwise cover a portion of tissue that is intact, to promote repair or to otherwise protect the underlying tissue.

### Y. Method of Use of Tissue Repair Device

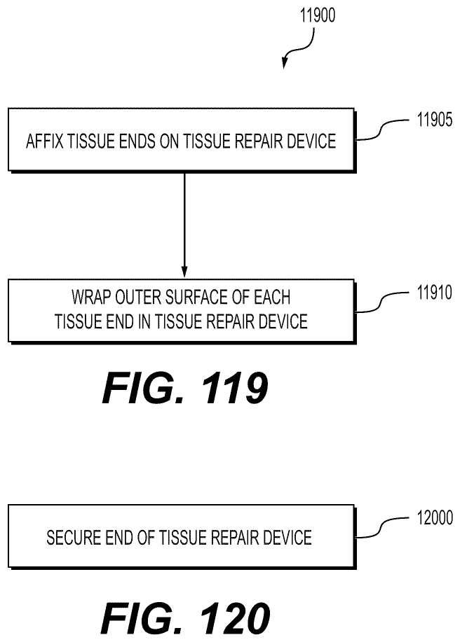

FIG. 119 is a flowchart of a method 11900 of securing two tissue ends together using a tissue repair device. The method may include a step 11905 of affixing the two tissue ends on a tissue repair device. Step 11905 may include orienting the tissue repair device so that the surface with barbs is facing the transected tissue ends. Step 11905 may then include affixing one portion of the barbs of the tissue repair device to a portion of a first transected tissue end region and attaching another portion of the barbs of the tissue repair device to a portion of a second transected tissue end region by engaging the barbs with outer surfaces of the transected tissue end regions.

In some embodiments, the step 11905 may also include aligning first and second transected tissue ends with each other on the tissue repair device and placement of the first and second transected tissue ends at a predetermined spacing from each other, to form a gap (e.g., any one of the gaps described herein) therebetween.

The method 11900 may also include a subsequent step 11910 of wrapping an outer surface of each of the two tissue ends in the tissue repair device. The step 11910 may include wrapping the tissue repair device, including a base membrane, according to some embodiments, around the outer surfaces of the first and second transected tissue ends, rolling the first and second transected tissue ends on the tissue repair device, or a combination thereof. In some embodiments, a separate base membrane may then also be wrapped around the tissue repair device.

The method 11900 may include affixing additional barbed sutures, barbed meshes, or barbed strips to the first and second tissue end regions as the tissue repair device is wrapped around the tissue surfaces.

The method 11900 may optionally also include a step 12000, shown in FIG. 120, of securing the ends of the tissue repair device, after the wrapping of the outer surfaces of the two tissue ends. The securing of the ends of the tissue repair device may include using one or more of a suture, an adhesive, a gel, or capillary action. The method 11900 may be part of a tissue repair process. In some embodiments, multiple tissue repair devices may be applied, or a singular tissue repair device may be applied. As described above with respect to several embodiments, a wrap may or may not be used as part of the tissue repair device.

In embodiments that use a base membrane, for example, a base membrane formed of SIS, a second layer of SIS base membrane may be provided on one end of a first layer of SIS base membrane, so that a portion of the first SIS base membrane may be wrapped around tissue ends one or more times, and then the end of the first base membrane having the second layer of SIS base membrane may be positioned against the underlying wrapped portion of the first layer of SIS base membrane, and the first base membrane and the second base membrane may remain in place via capillary effect.

In some embodiments, an additional layer of a gel may be applied to at least an end portion of a base membrane, so that a portion of the base membrane may be wrapped around tissue ends one or more times, and then the end of the base membrane having the gel thereon may be positioned against and stuck to the underlying wrapped portion of the base membrane.

A gap between the two tissue ends placed on the tissue repair device may be approximately 0 mm, or the gap may be greater than approximately 0 mm, for example, up to about 10 mm or up to about 5 mm. The gap may be greater than approximately 0 mm for the reasons set forth above with respect to gap G₆₇. In some aspects, the gap may be about 0 mm to about 10 mm, about 0 mm to about 8 mm, about 1 mm to about 10 mm, about 1 mm to about 8 mm, about 2 mm to about 8 mm, about 3 mm to about 7 mm, about 3 mm, about 4 mm, about 5 mm, about 6 mm, about 7 mm, etc.

The tissue repair device used in method 11900 may be any of the tissue repair devices shown and described in reference to FIG. 21-36, or 46-118, for example. In some aspects, the tissue repair device 6400, including the barbed mesh 6402 shown in FIGS. 64A to 67, or the tissue repair device 7000, including the base membrane 7002 and barbs 7005 shown in FIGS. 70 to 72, and may include at least one mesh including a plurality of barbed sutures, including a plurality of longitudinal barbed sutures and a plurality of lateral barbed sutures, each barbed suture including a suture body and at least one barb formed on an outer surface of the suture body, the at least one barb including at least one projection. Alternatively, the tissue repair device used in method 11900 may be, for example, the tissue repair device 7000, including the base membrane 7002 with barbs 7005, shown in FIG. 70, the plurality of barbs being formed by one of laser cutting and embossing. In addition, the tissue repair device may be the tissue repair device 7700, including the two barbed sutures 7702 and the base membrane 7705, the tissue repair device 7800, including the sets of welded, side-by-side barbed sutures 7802 and the base membrane 7805, shown in FIG. 78, the tissue repair device 7900, including the barbed strips 7902 and the base membrane 7905, shown in FIG. 79, or the tissue repair device 8100, including the barbed strips 8102 adhered to the strip of base membrane 8105 and the base membrane 8110, shown in FIG. 81. That is, the tissue repair device may include at least a wrap and at least two barbed sutures, a longitudinal length of the barbed sutures being greater than a width of the wrap, each barbed suture including portions including barbs and a portion that is free from barbs. By virtue of this method, two tissue ends may be secured together, to allow for a tissue repair process to occur.

For each of the configurations of tissue repair devices shown and described with respect to FIGS. 2 to 114, as an example, the transected tissue ends may be nerve ends, and the tissue repair process may be nerve regeneration. In addition, the method 11900 may be a method of securing two tissue ends together, and the tissue may be nerve tissue, with the transected tissue ends being transected nerve ends. In a case in which the tissue ends are nerve ends, barbs of the tissue repair devices of the embodiments described with respect to FIGS. 2 to 114 may protrude into or otherwise engage with the epineurium of each of the nerve ends, without extending into the perineurium or fascicles of each nerve end. Further, although transected tissue ends are discussed, the tissue repair devices of the embodiments described with respect to FIGS. 2 to 114 may be applied to injured tissues of any type, whether fully transected or not, e.g., for protection or to allow the underlying tissue to heal, or simply to secure tissue for any other suitable reason, unrelated to healing. For example, instead of two tissue ends being brought together, embodiments of the disclosure may wrap around or otherwise cover a portion of tissue that is intact, to promote repair or to otherwise protect the underlying tissue. The term ‘tissue ends’ as used herein should not be interpreted as limiting use of the devices, and the devices may be used in a variety of different ways and for procedures other than coaptation. Further, the tissue repair devices of the embodiments described with respect to FIGS. 2 to 114 may be used on tissue ends with a tissue graft being placed between the transected tissue ends.

### Z. Microscopic View of Etched Surface

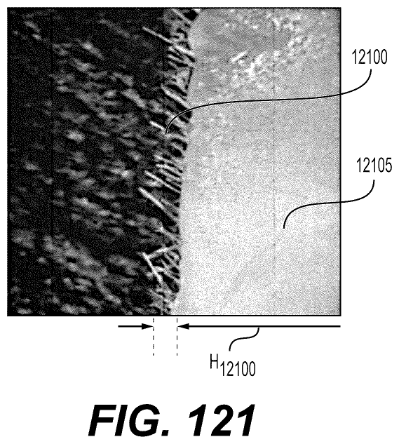

FIG. 121 is an image of glass fibers 12100 extending from a resorbable polymer surface 12105, the polymer surface having been etched back to expose the glass fibers 12100. The glass fibers 12100 may form microneedles of a height H₁₂₁₀₀, e.g., in a range of about 10 μm to about 500 μm, for example. The etching to expose glass fibers is an example of the forming of barbs or microneedles on a polymer surface, according to one or more embodiments described herein.

### AA. Benefits and Uses

Embodiments of the disclosure may allow for sutureless fixation of tissue, such as fixation of a nerve to other nerve ends or to a nerve adjunctive implant (e.g., protective covering, connector, transected nerve coaptation aid, drug delivery system), for example. In a case in which the barbed suture or the tissue repair device is used on a nerve, aspects of the disclosure may allow for secure attachment of the barbed suture or the tissue repair device to only an outer epineurial connective tissue and fatty tissue of the nerve, while leaving the internal nerve architecture (nerve fascicles, perineurium, endoneurium, and blood vessels) substantially undisturbed. For example, the penetration depth of the barb or projection, of the barbed suture or of the tissue repair device, in the vertical y-axis direction may not exceed about 150 microns. The length of cuts used to form the barbs, of the barbed suture or of the tissue repair device (if applicable), may be, e.g., up to about 250 microns, or longer, so long as the vertical penetration depth along the y-axis does not exceed about 150 microns. In some aspects, the angles of the barbs, of the barbed suture or of the tissue repair device (if applicable), may be up to about 45 degrees relative to the suture along its length or up to about 60 degrees relative to the suture along its length. Example sutures on which multi-directional barbs are included may be, e.g., 3-0 gauge or smaller, for example, 4-0 gauge, 5.0 gauge, 6.0 gauge, 7.0 gauge, or 8.0 gauge.

Embodiments of the disclosure may also afford benefits over existing methods for tissue fixation, such as placement of sutures or using no sutures, and over other forms of barbed sutures for the purpose of tissue fixation. Embodiments may promote improved fixation that resists against movement of the barbed suture in multiple directions. Embodiments may promote improved fixation that resists against movement of the tissue repair device and distributes the connective force of the tissue repair device across a broader surface(s) of the tissue(s) to which it is affixed. For example, sutures placed directly at a repair site to repair tissue put all of the force of retaining the tissue in place directly at the site of damage. By contrast, tissue repair devices of the current disclosure may distribute the force across a broader portion of the tissue(s) being repaired, which may reduce the amount of stress added to the site of repair.

Prototypes of the tissue repair devices prepared using methods disclosed herein have demonstrated the effectiveness of adhering sutures on a base membrane formed of SIS, in performing coaptation repair on transected nerve ends in a benchtop setting. The attachment of the sutures to the base membrane provides structural support to sutures and circumferential coverage at the coaptation site of tissue ends to inhibit the risk of axonal escape.

Although barbed sutures and tissue repair devices of the present disclosure are described for use with nerves, examples of tissue with which barbed sutures and tissue repair devices described herein may be used include nerve tissue, such as peripheral nerve tissue or central nervous system tissue, as well as other types of tissue. Exemplary tissue types suitable for use with barbed sutures and tissue repair devices of the present disclosure include, but are not limited to, epithelial tissue, connective tissue, muscular tissue, tendon tissue, ligament tissue, vascular tissue, intestinal tissue, dermal tissue, and cardiac tissue. The tissue may be mammalian tissue, including human tissue and tissue of other primates, rodent tissue, equine tissue, canine tissue, rabbit tissue, porcine tissue, or ovine tissue. In some aspects, the tissue may be non-mammalian tissue, selected from piscine, amphibian, or insect tissue. The tissue may be allogeneic or xenogeneic to a subject into which the graft is implanted. The tissue may be a synthetic tissue, such as, but not limited to, laboratory-grown or 3D-printed tissue.

It should be understood that although the present disclosure has been made with reference to preferred embodiments, exemplary embodiments, and optional features, modifications and variations of the concepts herein disclosed may be resorted to by those skilled in the art, and that such modifications and variations are considered to be within the scope of this disclosure as defined by the appended claims. The specific embodiments and examples provided herein are examples of useful embodiments of the present disclosure and are non-limiting and illustrative only. It will be apparent to one skilled in the art that the present disclosure may be carried out using a large number of variations of the devices, device components, methods, and steps set forth in the present description. As will be recognized by one of skill in the art, methods and devices useful for the present methods can include a large number of various optional compositions and processing elements and steps.

We claim:

1. A barbed suture comprising:
a suture body; and
at least one multi-directional barb formed on an outer surface of the suture body, the at least one multi-directional barb including a first projection and a second projection, wherein the first projection and the second projection extend in two different directions.

2. The barbed suture according to claim 1, wherein the at least one multi-directional barb is formed by:
making a first cut in the suture body, the first cut having a first depth and a first length and being formed at a first angle relative to a longitudinal axis of the suture body, and forming the first projection; and
making a second cut in the suture body, in a vicinity of the first cut, the second cut having a second depth and a second length and being formed at a second angle relative to the longitudinal axis of the suture body, and forming the second projection.

3. The barbed suture according to claim 2, wherein the first depth is less than the second depth.

4. The barbed suture according to claim 2, wherein the first angle is less than the second angle.

5. The barbed suture according to claim 2, wherein the first cut and the second cut overlap with respect to a radial axis of the suture body.

6. The barbed suture according to claim 1, wherein the at least one multi-directional barb includes a first barb and a second barb, and wherein at least one of the first barb and the second barb is one of:
hook-shaped;
a multi-directional barb having pointed projections;
a multi-directional barb having rounded projections; or
a geometrically-shaped barb.

7. The barbed suture according to claim 1, wherein the two different directions are angled relative to each other.

8. The barbed suture according to claim 1, wherein the two different directions are substantially opposite to each other.

9. The barbed suture according to claim 1, wherein the at least one multi-directional barb comprises a plurality of multi-directional barbs arranged in one of:
rows;
columns;
rings;
spirals, or
a geometric-shaped pattern on a surface of the suture body.

10. The barbed suture according to claim 1, wherein the at least one multi-directional barb comprises at least a first barb and a second barb, and wherein first projections of the first and the second multi-directional barbs are oriented in the same direction as each other, and second projections of the first and the second multi-directional barbs are oriented in the same direction as each other.

11. The barbed suture according to claim 1, wherein the at least one multi-directional barb comprises at least two barbs, and wherein the at least two barbs are arranged in a pattern, with first projections of a first subset of barbs pointing in a first direction and first projections of a second subset of barbs pointing in a second direction, different from the first direction.

12. A barbed suture comprising:
a suture body; and
at least two barbs formed on an outer surface of the suture body, a first barb of the at least two barbs having a first projection pointing in a first direction, and a second barb of the at least two barbs having a first projection pointing in a second direction, wherein the first direction is different than the second direction.

13. The barbed suture according to claim 12, wherein each of the first barb and the second barb is a uni-directional barb.

14. The barbed suture according to claim 12, wherein each of the first barb and the second barb is a multi-directional barb.

15. The barbed suture according to claim 12, wherein at least one of the first barb and the second barb is one of:
hook-shaped;
a multi-directional barb having pointed projections;
a multi-directional barb having rounded projections;
a circular disc;
a single, rounded barb; or
a geometrically-shaped barb.

16. The barbed suture according to claim 12, wherein the at least two barbs are arranged in one of:
rows;
columns;
rings;
spirals, or
a geometric-shaped pattern on a surface of the suture body.

17. The barbed suture according to claim 12, wherein the at least two barbs are formed on a portion of a length of the suture body, and no barbs are formed on another portion of the length of the suture body.

18. The barbed suture according to claim 12, wherein the at least two barbs are formed on a portion of a circumference of the suture body, and no barbs are formed on another portion of the circumference of the suture body.

19. The barbed suture according to claim 12, wherein the at least two barbs are oriented so that they are angled relative to a longitudinal axis of the suture body.

20. A method of making a barbed suture, the method comprising:
making a first cut into a suture body to form a first projection oriented in a first direction, wherein the first cut does not extend fully through the suture body; and
making a second cut into the suture body to form a second projection oriented in a second direction, wherein the second cut does not extend fully through the suture body,
wherein the first direction is different from the second direction.

21. A tissue repair device comprising:
at least one mesh including:
a plurality of barbed sutures, including a plurality of longitudinal barbed sutures and a plurality of lateral barbed sutures, each barbed suture including:
a suture body; and
at least one barb formed on an outer surface of the suture body.

22. The tissue repair device according to claim 21, wherein the plurality of longitudinal barbed sutures and the plurality of lateral barbed sutures are arranged in a grid pattern.

23. The tissue repair device of claim 21, further comprising a base membrane configured to overlay a non-barbed surface of the plurality of barbed sutures during use.

24. The tissue repair device of claim 21, further comprising sheet of a base membrane attached to a non-barbed surface of the plurality of barbed sutures.

25. The tissue repair device of claim 21, wherein the plurality of sutures includes two or more lateral sutures and two or more longitudinal sutures, the two or more lateral sutures having a relatively greater length than the two or more longitudinal sutures, thus forming at least one strip of mesh.

26. The tissue repair device of claim 25, further comprising at least one sheet of base membrane to which the at least one strip of mesh is attached.

27. The tissue repair device of claim 26, wherein the at least one strip of mesh includes a portion free from barbs, located in a longitudinal center region of the mesh.

28. The tissue repair device of claim 26, further comprising a base membrane attached to a non-barbed surface of the strip of mesh.

29. The tissue repair device of claim 21, further comprising a base membrane, wherein a longitudinal length of each lateral barbed suture is greater than a width of the base membrane, wherein the mesh includes a portion that is free from barbs located in a longitudinal center of each lateral barbed suture, and wherein the base membrane extends over the portion of the mesh that is free from barbs.

30. A tissue repair device comprising:
a sheet of base membrane; and
a plurality of barbs formed on the sheet of base membrane, the plurality of barbs being formed by one of laser cutting or embossing.

31. A tissue repair device comprising:
a base membrane; and
a plurality of barbs formed on the base membrane, the plurality of barbs being formed of resorbable ceramic and being attached to the base membrane.

32. A tissue repair device comprising:
a base membrane; and
at least two barbed sutures, a longitudinal length of each of the at least two barbed sutures being greater than a width of the base membrane, each barbed suture including portions having barbs and a portion that is free from barbs.

33. The tissue repair device of claim 32, wherein the at least two barbed sutures are spaced apart from each other and are woven through the base membrane, such that the portion of each barbed suture that is free from barbs is woven through the base membrane, and the portions of each barbed suture that have barbs extend outward from the base membrane.

34. The tissue repair device of claim 32, wherein the base membrane includes at least two pre-made holes, and wherein the at least two barbed sutures include at least one set of welded barbed sutures, the at least one set of barbed sutures passing through the at least two pre-made holes in the base membrane.

35. The tissue repair device of claim 32, wherein the at least two barbed sutures includes at least one strip of barbed sutures, arranged in a thin grid pattern.

36. A tissue repair device comprising:
a strip of base membrane; and
at least two barbed sutures attached to the strip of base membrane.

37. The tissue repair device of claim 36, wherein each barbed suture includes portions having barbs and a portion that is free from barbs.

38. The tissue repair device of claim 36, wherein the at least two barbed sutures are arranged to form a mesh.

39. A method of securing two tissue ends relative to one another, the method comprising:
affixing the two tissue ends to a tissue repair device comprising at least one mesh formed of a plurality of barbed sutures, wherein the plurality of barbed sutures includes a plurality of longitudinal barbed sutures and a plurality of lateral barbed sutures, each barbed suture including a suture body and at least one barb formed on an outer surface of the suture body; and
wrapping an outer surface of each of the two tissue ends in the tissue repair device.

40. The method of claim 39, wherein a gap between the two tissue within the tissue repair device is between 0 mm and about 5 mm.

41. The method of claim 39, further comprising securing an end of the tissue repair device after the wrapping of the outer surfaces of the two tissue ends.

42. The method of claim 41, wherein the securing includes using one or more of a suture, a suture, and an adhesive.

43. The method of claim 39, wherein the method further comprises wrapping a base membrane around the tissue repair device to cover the tissue repair device.

44. The method of claim 43, wherein the wrapping the base membrane occurs at the same time as wrapping the tissue repair device.

45. The method of claim 43, wherein the wrapping the base membrane occurs after wrapping the tissue repair device.

46. The method of claim 39, wherein the tissue repair device further comprises a base membrane to which the at least one mesh is attached.

47. The method of claim 39, wherein the at least one mesh is at least one strip of mesh, and the plurality of barbed sutures includes two lateral barbed sutures and two or more longitudinal barbed sutures, the two lateral barbed sutures having a relatively greater length than the two longitudinal barbed sutures.

48. The method of claim 47, further comprising at least one base membrane on which the at least one strip of mesh is attached.

49. The method of claim 48, wherein the at least one strip of mesh includes a portion free from barbs, located in a longitudinal center region of the mesh.

50. A method of securing two tissue ends together, the method comprising:
affixing the two tissue ends to a tissue repair device comprising a base membrane and a plurality of barbs formed on the base membrane, the plurality of barbs being formed by one of laser cutting and embossing; and
wrapping an outer surface of each of the two tissue ends in the tissue repair device.

51. A method of securing two tissue ends together, the method comprising:
affixing the two tissue ends to a tissue repair device comprising a base membrane and a plurality of barbs attached to the base membrane, the plurality of barbs being formed of resorbable ceramic; and
wrapping an outer surface of each of the two tissue ends in the tissue repair device.

52. A method of securing two tissue ends together, the method comprising:
affixing the two tissue ends to a tissue repair device comprising a base membrane and at least two barbed sutures, a longitudinal length of the barbed sutures being greater than a width of the base membrane, each barbed suture comprising portions including barbs and a portion that is free from barbs; and
wrapping an outer surface of each of the two tissue ends in the tissue repair device.

53. The method of claim 52, wherein the at least two barbed sutures are spaced apart from each other and are woven through the base membrane, such that the portion of each barbed suture that is free from barbs is woven through the base membrane, and the portions of each barbed suture that include barbs extend outward from the base membrane.

54. The method of claim 52, wherein the base membrane includes at least two pre-made holes, and wherein the at least two barbed sutures include at least one set of welded barbed sutures, the at least one set of barbed sutures passing through the at least two pre-made holes in the base membrane.

55. The method of claim 52, wherein the at least two barbed sutures includes at least one strip of barbed sutures, arranged in a grid pattern.

56. The method of claim 55, further comprising at least one strip of base membrane to which one side of the at least one strip of barbed sutures is attached.
